(19) United States
(12) Patent Application Publication
(10) Pub. No.: US 2024/0075065 A1
(43) Pub. Date: Mar. 7, 2024

(54) CD28-TARGETING CHIMERIC ANTIGEN RECEPTOR (CAR) T CELLS, METHODS OF GENERATION AND USES THEREOF

(71) Applicant: LUDWIG-MAXIMILIANS-UNIVERSITÄT MÜNCHEN, München (DE)

(72) Inventors: Tobias FEUCHTINGER, München (DE); Semjon WILLIER, München (DE)

(21) Appl. No.: 18/321,344

(22) Filed: May 22, 2023

Related U.S. Application Data
(63) Continuation-in-part of application No. PCT/EP2021/082713, filed on Nov. 23, 2021.

(30) Foreign Application Priority Data
Nov. 23, 2020 (EP) 20209327.4

(51) Int. Cl. A61K 35/17; A61K 39/00; A61K 45/06; A61P 35/02; C07K 16/28; C12N 5/0783; C12N 15/86

(52) U.S. Cl. CPC A61K 35/17; A61K 39/4611; A61K 39/4631; A61K 39/464429; A61K 45/06; A61P 35/02; C07K 16/2818; C12N 5/0636; C12N 15/86; A61K 2239/13; A61K 2239/21

(57) ABSTRACT

The present invention relates to a modified T cell, comprising (a) a disrupted endogenous CD28-encoding gene; and (b) a polynucleotide encoding a chimeric antigen receptor (CAR), wherein the CAR comprises in its ectodomain at least one antigen binding moiety that is capable of specific binding to the extracellular portion of CD28. The invention furthermore relates to a population of the modified T cells, to a method for generating modified T cells and medical and non-medical uses thereof.

### CROSS-REFERENCE TO RELATED APPLICATIONS

The present application is a continuation-in-part of PCT Patent Application No. PCT/EP2021/082713 (published as WO/2022/106731) which was filed on Nov. 23, 2021, together with a sequence listing, and which claims the benefit of priority from European patent application EP 20 209 327.4 which was filed on Nov. 23, 2020, together with a sequence listing. The entire contents of each of the above-identified applications and sequence listings are incorporated herein by reference in their entireties.

### SEQUENCE LISTING

The instant application contains a Sequence Listing which has been submitted electronically in XML format and is hereby incorporated by reference in its entirety. Said XML copy, created on May 11, 2023, is named 4_sequence_listing and is 113,614 bytes in size.

### FIELD

The present invention relates to a modified T cell, comprising (a) a disrupted endogenous CD28-encoding gene; and (b) a polynucleotide encoding a chimeric antigen receptor (CAR), wherein the CAR comprises in its ectodomain at least one antigen binding moiety that is capable of specific binding to the extracellular portion of CD28. The invention furthermore relates to a population of the modified T cells, to a method for generating modified T cells and medical and non-medical uses thereof.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### BACKGROUND

Treatment with chimeric antigen receptor (CAR) T cells represents a new paradigm in immunotherapy of cancer.⁶ Engineering primary T cells was first presented more than 30 years ago.¹⁷ Since then, chimeric antigen receptor T cells have emerged as a promising technique for treatment of relapsed/refractory B-cell malignancies. In particular, CAR treatments have shown high initial complete response rates in patients with relapsed and refractory B-lineage malignancies like acute B-precursor leukemia (BCP-ALL) and non-Hodgkin lymphoma.^{7, 8, 9, 10} Depletion of B-cells through anti-CD19 CARs is easily compensated by infusion of immunoglobulins. Anti-CD19-CAR T cell therapy has been approved by the FDA and EMA since 2018.

However, malignancies of T-cell lineage, such as acute lymphoblastic leukemia or lymphomas, are currently excluded from these new treatments. Only few studies have evaluated CAR T cell therapy for the treatment of T-cell lineage malignancies.^{11, 12} Harnessing and redirecting the cytotoxicity of T cells selectively to malignant T cells while sparing physiologic T cells is complex and faces severe challenges:

First, suitable target antigens that are exclusively expressed on malignant T cells and absent on normal T cells are unknown. Targeting of an antigen also regularly expressed on normal T cells would lead to T cell aplasia and culminate in profound immune deficiency, likely associated with high rates of morbidity and mortality.

Second, expression of the targeted antigen on CAR T cells itself results in fratricide, precluding expansion of such CAR-modified T cells already during manufacture.

Third, the few T cell surface antigens which were identified to having only limited expression on normal T-cells subsets have only little or no functional relevance for the cell. To date, only a few surface antigens with poor expression on normal T cells have been assessed as CAR targets (TRBC1, CD30, CD37, and CD1a) to treat T-cell malignancies. However, since their expression is limited to only small subsets of T-cell malignancies and absent on most T-ALLs, their suitability as targets for CAR-T cell therapy is hampered, particularly in conditions like pediatric T-ALL.

T-ALL is a heterogeneous disease, characterized by overexpression of CD7 and frequent expression of other T-cell markers such as CD2 and CD5. However, these surface markers, though being T-lineage specific, are universally expressed on precursor and mature T cells, not limited to malignant T-cells. Thus, targeting CD2, CD5 or CD7 by CAR T-cells also depletes the majority of physiologic T cells and T cell precursors, inducing a severe combined immunodeficiency (SCID)-like immune status.^{12, 18-20} While both anti-CD5 and anti-CD7 CAR T-cells have been described in preclinical models and early phase clinical studies, no data on clinical efficacy is currently available.^{11, 12} Especially anti-CD7 CARs will deplete a wide range of T-cell precursors, since CD7 is expressed already early in lymphopoiesis as the first antigen expressed in cells committed to the T-cell lineage.²¹ Moreover, because CD5 and CD7 have no known functional relevance for malignant cells, immune escape mechanisms resulting in a target-downregulation or -disappearance are likely, similar as has been reported for B-cell targeting CD19-CAR T cell therapy.²²

At present, the only clinically available therapy for patients with relapsed/refractory T-cell malignancies is an allogeneic stem cell transplant, which, however, has its own associated risks, such as graft-versus-host disease (GVHD), viral infection, and/or toxicities.

Thus, there is an urgent need in the art for alternative, more effective and safer means for treating T-cell mediated disorders and other disorders in which T cells contribute to disease pathology. The present invention addresses this need and provides means and methods useful for such treatments.

### SUMMARY AND DETAILED DESCRIPTION

Accordingly, the present invention relates in a first aspect to a modified T cell, comprising (a) a disrupted endogenous CD28-encoding gene; and (b) a polynucleotide encoding a chimeric antigen receptor (CAR), wherein the CAR comprises in its ectodomain at least one antigen binding moiety that is capable of specific binding to the extracellular portion of CD28.

“T cells” or “T lymphocytes”, as interchangeably referred to herein, are a subset of lymphocytes (a subtype of white blood cell) that originate from hematopoietic stem cells produced in the bone marrow and migrate to the thymus for maturation and play a central role in cell-mediated immunity. T cells can be distinguished from other lymphocytes, such as B cells and natural killer (NK) cells, e.g., by the presence of a T-cell receptor (TCR) on their cell surface. T cells include, for example, natural killer T (NKT) cells, cytotoxic T lymphocytes (CTL), T helper (Tₕ) cells, and regulatory T (T_{reg}) cells.

The term “CD28” refers to the receptor “cluster of differentiation 28”. CD28 (also known as “TP44”) is a 44 kDa type I transmembrane surface glycoprotein predominantly expressed on T cells. CD28 is the receptor for the CD80 (B7.1) and CD86 (B7.2) proteins, which are expressed on activated B cells and antigen-presenting cells (APCs). Ligation of the CD28 receptor on T cells provides a critical co-stimulatory signal (also termed “second” signal) alongside T cell receptor (TCR) ligation for T cell activation. Earlier work has shown that T cell receptor (TCR)-ligation alone is not sufficient for T cell activation and even leads to T cell anergy and unresponsiveness, and that the necessary second or “co-stimulatory” signal that prevents T cell unresponsiveness after TCR ligation can be provided by CD28. CD28-ligation drives critical intracellular biochemical events including unique phosphorylation and transcriptional signaling, metabolism, and the production of key cytokines, chemokines, and survival signals that are essential for long-term expansion and differentiation of T cells. CD28 is therefore believed to be essential for the coordination of the adaptive immune response and maintenance of immune homeostasis. The CD28-encoding gene is composed of four exons encoding a protein of 220 amino acids that is expressed on the cell surface as a glycosylated, disulfide-linked homodimer of 44 kDa. Each exon defines an individual functional domain of the protein: exon 1 encodes the signal peptide, exon 2 encodes the extracellular domain, exon 3 encodes the transmembrane region, and exon 4 encodes the cytoplasmic region. The structural and functional properties of CD28 are reviewed for example in Esensten J H, et al., Immunity (2016); 44(5):973-88.

The nucleotide sequences of the CD28-encoding gene (including the intron/exon structure) in different mammalian species and the amino acid sequences of the respectively encoded proteins are known in the art and can be retrieved from publicly available databases such as the NCBI database (https://www.ncbi.nlm.nih.gov/) or UniProt (https://www.uniprot.org/). For example, the nucleotide sequence of the
human CD28-encoding gene is defined in NCBI Reference Sequence: NG_029618.1;
murine CD28-encoding gene is defined in NCBI Reference Sequence: NM_007642.4;
rat CD28-encoding gene is defined in NCBI Reference Sequence: NM_013121.1.

The term “disrupted endogenous CD28-encoding gene”, as used herein, means that the endogenous CD28-encoding gene is genetically modified, e.g., as a result of targeted gene editing, in a way that the functional expression of at least the extracellular portion of the endogenous CD28 protein is prevented or disturbed so that the modified T cell having that disrupted endogenous CD28-encoding gene will not be able to being bound (i.e. recognizable) by the CAR which comprises in its ectodomain at least one antigen binding moiety that is capable of specific binding to the extracellular portion of CD28 (e.g., of CD28 expressed on other cells than the modified T cell (e.g., malignant T cells or malignant B cells)). Means and methods for targeted gene editing are known in the art and further described herein below.

The term “nucleic acid” or “polynucleotide” refers to deoxyribonucleic acids (DNA), such as cDNA or genomic DNA, and, where appropriate, ribonucleic acids (RNA) and polymers thereof in either single- or double-stranded form. It is understood that the term “RNA” as used herein comprises all forms of RNA including mRNA.

The term “(poly)peptide” as used herein in its broadest sense refers to a group of molecules which comprises the group of “peptides”, as well as the group of “polypeptides”, the latter term being interchangeably used with the term “protein”. The group of “peptides” consists of molecules up to 30 amino acids, the group of “polypeptides” consists of molecules with more than 30 amino acids. The group of “peptides” also refers to fragments of proteins of a length of 30 amino acids or less. (Poly)peptides may further form dimers, trimers and higher oligomers, i.e., consisting of more than one (poly)peptide molecule. (Poly)peptide molecules forming such dimers, trimers etc. may be identical or non-identical. The corresponding higher order structures are, consequently, termed homo- or heterodimers, homo- or heterotrimers etc. Homo- or heterodimers etc. also fall under the definition of the term “(poly)peptide”. The term “(poly)peptide” also refers to chemically or post-translationally modified peptides and polypeptides.

The term “chimeric antigen receptor (CAR)”, as used herein, generally refers to an artificial immune cell receptor that is engineered to bind specifically to a target antigen expressed by one or more target cells, e.g., a particular cell surface molecule on a malignant cell (e.g., a malignant T cell). Generally, a CAR is designed for being expressed by an immune cell, e.g., a T cell, and is a chimera of an extracellular antigen binding moiety (e.g., a single-chain fragment variable (scFv) or other antibody fragment) that mediates specific antigen recognition/binding and an intracellular signaling domain that upon extracellular antigen engagement provides T cell stimulatory signaling. Typically, a CAR is expressed as a type I transmembrane protein, although other configurations are possible and envisaged herein. A T cell that expresses a CAR is referred to as a CAR T cell. CARs have the ability to redirect T-cell specificity and reactivity toward a selected target in a non-MHC-restricted manner. The non-MHC-restricted antigen recognition confers T-cells expressing CARs the ability to recognize an antigen independent of antigen processing, thus bypassing a major mechanism of immune escape of malignant cells. It is generally to be understood that a modified T cell according to the invention which, as defined in accordance with the herein disclosed invention, comprises a polynucleotide encoding a CAR, is capable of functionally expressing, preferably functionally expresses, said CAR also on the protein level.

A CAR typically comprises an “extracellular domain” (also referred to herein as “ectodomain”) having at least one antigen binding moiety, a “transmembrane domain”, and an “intracellular domain” (also referred to herein as “endodomain” or “cytoplasmic domain”). It is understood that the term “domain”, as used herein, is not necessarily limited to its narrower meaning as commonly used in structural biology to only consist of a single structural protein domain, but may according to its herein used broader interpretation also refer to a region or portion of a protein which comprises more than one structural protein domain.

The ectodomain is the portion of the CAR that is exposed to the extracellular space and comprises at least one antigen binding moiety, and optionally one or more hinge regions. Further components may additionally be comprised in the ectodomain as discussed below. In some instances, the antigen binding moiety is a fragment of an antibody, preferably a single-chain fragment variable (scFv).

The term “antigen binding moiety”, as interchangeably used herein with the terms “antigen binding molecule”, “antigen binding agent” or “antigen binding domain”, refers to a molecular structure that binds specifically to a predefined target antigen (e.g., protein/peptide, lipid, DNA, RNA, carbohydrate, and/or a portion, modification or combination thereof). Non-limiting examples of an antigen binding moiety include antibodies (e.g., antibodies immunologically or genetically derived from any species (e.g., human, chicken, camel, llama, lamprey, shark, goat, rodent, cow, dog, rabbit, etc.), antibody fragments, domains or parts thereof (e.g., Fab, Fab′, F(ab′)₂, scFab, Fv, scFv, VH, VHH, VL, VLRs, the like), diabodies, monoclonal antibodies, polyclonal Abs, mAbdAbs, phage display derived binders, affibodies, heteroconjugate antibodies, bispecific antibodies, evibodies, lipocalins, anticalins, affibodies, avimers, maxibodies, heat shock proteins such as GroEL and GroES, trans-bodies, DARPins, aptamers, C-type lectin domains (e.g., tetranectins); human γ-crystallin and human ubiquitin-derived binders (e.g., affilins), PDZ domain-derived binders; scorpion toxin and/or Kunitz-type domain binders, fibronectin-derived binders (e.g., adnectins), receptors, ligands, lectins, streptavidin, biotin, including derivatives and/or combinations thereof (such as bi-/multi-specific formats formed from two or more of these binding molecules). Various antibody-derived and alternative (i.e., non-antibody) binding protein scaffolds including methods of generation thereof are known in the art (e.g., reviewed in Chiu M L et al., Antibodies (Basel), (2019); 8(4):55; Simeon R. & Chen Z., Protein Cell. (2018); 9(1):3-14; and Chapter 7—Non-Antibody Scaffolds from Handbook of Therapeutic Antibodies (2007) edited by Stefan Dübel).

“Evibodies”, in accordance with the present invention, are engineered binding proteins derived from the variable(V)-set Ig-like scaffold of the T-cell surface receptor Cytotoxic T Lymphocyte-associated Antigen 4 (CTLA-4). Loops corresponding to CDRs of antibodies can be substituted with heterologous sequences to confer different binding properties. Methods of making Evibodies are known in the art and are described, for example, U.S. Pat. No. 7,166,697.

“Lipocalins”, in accordance with the present invention, are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. “Anticalins” (also termed “Affilins”), in accordance with the present invention, are between 160-180 amino acids in size, and are derived from lipocalins (Rothe C & Skerra A, BioDrugs. (2018); 32(3):233-243; Gebauer M & Skerra A, Curr Opin Biotechnol. (2019); 60:230-241).

“Affibodies”, in accordance with the present invention, are a family of antibody mimetics that is derived from the Z-domain of staphylococcal protein A. Affibodies are structurally based on a three-helix bundle domain. An affibody has a molecular mass of around 6 kDa and is stable at high temperatures and under acidic or alkaline conditions. Target specificity is obtained by randomization of amino acids located in two alpha-helices involved in the binding activity of the parent protein domain (Feldwisch, J & Tolmachev, V. (2012) Methods Mol. Biol. 899:103-126). Methods of making affibodies are known in the art and are described, e.g., in Wikman M et al., Protein Eng Des Sel. (2004); 17(5):455-62.

“Avimers” (short for avidity multimers), in accordance with the present invention, are a class of artificial multi-domain proteins which specifically bind certain antigens via multiple binding sites. This protein is also known as “maxibody” or low-density lipoprotein receptor (LDLR) domain A. It consists of two or more (poly)peptide sequences, which are based on A domains. The A domains are 30 to 35 amino acids scaffolds (˜4 kDa) derived from extracellular cysteine-rich cell surface receptor proteins and stabilized by disulfide bond formation and complexation of calcium ions. The scaffold structure is maintained by 12 conserved amino acids, leaving all the remaining non-conserved residues amenable to randomization and ligand binding. Avimers are highly thermostable. Due to their small size, avimers often consist of multiple A-domains with each binding to a different site on the target, thereby achieving increased affinity through avidity (Silverman J et al. (2005), Nat Biotechnol 23:1556-1561).

“DARPins”, in accordance with the present invention, are designed ankyrin repeat domains and based on tightly packed ankyrin repeats, each forming a β-turn and two antiparallel α-helices. DARPins usually carry three repeats corresponding to an artificial consensus sequence, whereby a single repeat typically consists of 33 amino acids, six of which form the binding surface. During recombinant library design, these sites are used to introduce the codons of random amino acids. DARPins are typically formed by two or three of the binding motifs contained between the N- and C-terminal motifs shielding the hydrophobic regions. DARPins are small proteins (˜14-18 kDa) that are extremely thermostable and resistant to proteases and denaturing agents (Pluckthun A., Annu Rev Pharmacol Toxicol. (2015); 55:489-511).

“Kunitz-type domain binders”, in accordance with the present invention, are ˜60-amino-acid polypeptides (˜7 kDa) derived from the active motif of Kunitz-type protease inhibitors such as aprotinin (bovine pancreatic trypsin inhibitor), Alzheimer's amyloid precursor protein, and tissue factor pathway inhibitor. The hydrophobic core of the Kunitz domain is composed of a twisted two-stranded antiparallel β-sheet and two α-helices stabilized by three pairs of disulfide bonds. Residues in the three loops can be substituted without destabilizing the structural framework (Hosse R J et al. (2006). Protein Sci 15:14-27; Simeon R. & Chen Z. Protein Cell. (2018); 9(1):3-14).

“Adnectins”, in accordance with the present invention, is a class of binding proteins having a scaffold which consists of a backbone of the natural amino acid sequence of the 10th domain of the 15 repeating units of human fibronectin type Ill (FN3). The molecule adopts a β-sandwich fold with seven strands connected by six loops similar like an immunoglobulin domain, but without any disulfide bonds. Three loops at one end of the β-sandwich can be engineered to enable an adnectin to specifically recognize a therapeutic target of interest. Non-loop residues have also been found to expand the available binding footprint. Ligand-binding adnectin variants with binding affinities in the nanomolar to picomolar range have been selected via mRNA, phage, and yeast display (Hackel B J, et al. (2008) J Mol Biol 381:1238-1252).

Means and methods for developing, screening and identification of suitable binding molecules (e.g., (poly)peptides of various scaffolds, including, without being limiting, those described herein above) toward desired target structures (such as the CD28 extracellular domain) are well known and routinely employed in the art. Exemplary nowadays routinely-performed methods include, without intended to being limiting, high-throughput (HT) combinatorial library-based display and selection methods, such as phage display, ribosome display, mRNA display, and cell surface display (e.g., yeast display).

In accordance with the present invention, antibody fragments comprise, inter alia, Fab or F(ab′) fragments, F(ab′)₂, Fv or scFv fragments, single domain VH, VL or V-like domains, such as VHH or V-NAR-domains, as well as multimeric formats such as minibodies, diabodies, tribodies, triplebodies, tetrabodies or chemically conjugated Fab′-multimers (see, for example, Holliger, P. & Hudson, P. J. (2005) Nat. Biotechnol. 23:1126-1136).

A scFv includes a light chain variable domain (VL) and a heavy chain variable domain (VH) of immunoglobins which are connected by a short linker peptide (referred to herein as “linker”) to form a single polypeptide chain. A scFv may be in a configuration where the linker is between the carboxy(C)-terminal amino acid residue of the VL and the amino(N)-terminal amino acid residue of the VH, or between the C-terminal amino acid residue of the VH and the N-terminal amino acid residue of the VL.

As has been observed by the present inventors, either of these configurations (VL-linker-VH or VH-linker-VL, respectively) can provide functional constructs, albeit, depending on the individual VL and VH domains, either the one or the other configuration can be more suitable. For example, constructs having a CAR with a scFv with VL and VH domains from the anti-human CD28 IgG4 antibody “TGN1412” (reviewed in Beyersdorf N et al., Immunotargets Ther. (2015); 4:111-22), while being functional in either configuration (cf. constructs 1, 2, 11 and 12 in Table 1 of Example 2), proved to be particularly effective when having a VL-linker-VH configuration (constructs 1 and 2). Constructs having a CAR with a scFv with VL and VH domains from the monoclonal anti-human CD28 IgG1 antibody “CD28.3” (Vanhove B et al., Blood. (2003); 102(2):564-70) were effective when having a VH-linker-VL configuration (construct 14 in Table 1). Thus, in those instances where the VH and VL domains of the scFv derive from the TGN1412 antibody, a VL-linker-VH configuration of the scFv is particularly preferred; whereas in other instances where the VH and VL domains of the scFv derive from the CD28.3 antibody, a VH-linker-VL configuration of the scFv is preferred.

However, it is generally contemplated herein that either of the aforementioned configurations (VL-linker-VH or VH-linker-VL) can be employed in accordance with the invention, and it is within the common abilities of the skilled person to test and select the most suitable configuration, for example, by assaying (in vitro) and comparing the cytotoxic capacity of different modified T cells toward CD28-expressing target cells.

Any peptide having sufficient flexibility and length can be used as a linker in a scFv. Usually, the linker is selected to have little to no immunogenicity. In some embodiments, the linker includes glycine and/or serine. A linker is typically a peptide of 10 to 30 amino acids in length, preferably of 15 to 20 amino acids in length. For example, a linker may have the amino acid sequence (GGGGS (SEQ ID NO: 1))ₙ, where n is 2, 3, 4, 5 or 6, preferably 3 or 4. Other linker sequences and lengths can also be used.

In a preferred embodiment, the linker comprises or consists of the amino acid sequence GSTSGSGKPGSGEGSTKG (SEQ ID NO: 2). This linker sequence (also known as “Whitlow linker”) has been reported to provide reduced aggregation and to be particularly stable to proteolysis (Whitlow M, et al. Protein Eng. (1993); 6(8):989-95).

In another preferred embodiment, the CAR further comprises a “signal peptide”, wherein the signal peptide is at the amino(N)-terminus of the CAR. The main function of a “signal peptide”, hereinafter also referred to interchangeably as “signal sequence”, “leader sequence” or “leader peptide”, is to provide a sorting signal which directs the expression of the CAR via the secretory pathway (during which the signal peptide gets proteolytically removed) to the surface of the T cell. A signal peptide can be found at the N-terminus of many secretory proteins and membrane proteins and typically has a length of 15 to 30 amino acids. Signal peptides from any such secretory protein and/or membrane protein may be used for a CAR of the present disclosure. Signal peptides can be from a mammalian species and can also be derived from non-mammalian species, for example, from insects, yeast, bacteria or viruses. The signal peptide is preferably a mammalian signal peptide, more preferable a human signal peptide. Exemplary signal peptides include, without limitation, the signal peptide of CD8 alpha, e.g., the human CD8 alpha signal peptide (MALPVTALLLPLALLLHAARP (SEQ ID NO: 3)), or a signal peptide from an immunoglobulin (such as an IgG heavy chain or IgG light chain). Further signal peptides are known in the art and described, e.g., in Hegde R S & Bernstein H D, Trends Biochem Sci. 2006; 31(10):563-71.

In a particularly preferred embodiment, the signal peptide comprises or consists of the amino acid sequence MLLLVTSLLLCELPHPAFLLIP (SEQ ID NO: 4; corresponding to the signal peptide of human colony stimulating factor 2 receptor subunit alpha (CSF2RA)).

In some embodiments, the CAR further comprises one or more hinge regions between the antigen binding moiety and the transmembrane domain, and/or between the transmembrane domain and the cytoplasmic signaling domain. In cases where the CAR comprises more than one antigen binding moiety and/or more than one cytoplasmic signaling domain, one or more hinge regions may also be present between individual antigen binding moieties and/or between individual cytoplasmic signaling domains.

A hinge region may be incorporated into the CAR in order to provide structural flexibility and/or to prevent steric hindrance of the CAR or individual structural domains thereof. For example, a hinge region may be incorporated into the extracellular portion of the CAR in order to provide structural flexibility to the antigen binding moiety to access the targeted antigen (or a particular epitope on that antigen). Studies have shown that the optimal hinge length of a given CAR depends on the position of the targeted epitope. Long hinges provide extra flexibility to the CAR and allow for better access to membrane-proximal epitopes or complex glycosylated antigens, whereas CARs bearing short hinges are more effective at binding membrane-distal epitopes; see Review by Guedan S et al., Mol Ther Methods Clin Dev. (2018); 31; 12:145-156.

In some embodiments, a hinge region may comprise up to 250 amino acids (e.g., 5 to 100 amino acids, or preferably 10 to 65 amino acids, or more preferably 15 to 55 amino acids). In the enclosed working examples, CARs having a short hinge region (15 amino acids in length) and CARs having a long hinge region (55 amino acids in length) between the antigen binding moiety and the transmembrane domain proved to be effective in terms of engagement and elimination of target cells, whereby CARs having a long hinge region (55 amino acids in length) were performing best. It is thus expected that hinges of any length between 15 and 55 amino acids are particularly suitable. Hinge regions of shorter or longer lengths than 15 to 55 amino acids may also be suitable.

Accordingly, in preferred embodiments, the CAR comprises a hinge region between the (most membrane proximal) antigen binding moiety and the transmembrane domain, wherein the hinge region comprises or consists of 45 to 65 amino acids, more preferable of 50 to 60 amino acids, even more preferable of 52 to 58 amino acids, and most preferably of 55 amino acids. In a preferred embodiment, the CAR comprises a hinge region between the (most membrane proximal) antigen binding moiety and the transmembrane domain, wherein the hinge region is preferably derived from the amino acid sequence of CD8, preferably from the amino acid sequence of the CD8 alpha chain, more preferably from the extracellular region of the CD8 alpha chain.

In a particularly preferred embodiment, the hinge region comprises or consists of a polypeptide having the amino acid sequence of the CD8 alpha extracellular portion, preferably of the human CD8 alpha extracellular portion as defined by SEQ ID NO: 5:

FVPVFLPAKPTTTPAPRPPTPAPTIASQPLSLRPEACRPAAGGAVHTRG
LDFACD.

Alternatively, the hinge region may comprise or consist of an amino acid sequence derived from the non-variable domains of an immunoglobulin G (IgG) (IgG1, IgG2 or IgG4), preferably a single CH3 domain.

In another alternative embodiment, the hinge region comprises or consists of the amino acid sequence (GGGGS (SEQ ID NO: 1))ₙ, where n is 2, 3, 4, 5, or 6, preferably 3 (as defined by SEQ ID NO: 6).

The ectodomain of the CAR may further comprise an epitope-tag, a (poly)peptide (“tag”) which provides a binding epitope for tag-specific binding agents (e.g., an antibody) that can be used, for example, for assessing CAR expression on the T cell(s). The incorporation of an epitope-tag in the ectodomain of the CAR can thus be useful for assessing the transduction rate of the CAR T cells, e.g., by comparison to untransduced T cells. In addition, the incorporation of an epitope-tag in the ectodomain of the CAR may be exploited for tracking, selection (i.e., selective enrichment/purification) or depletion of CAR-expressing T cells (e.g., in vivo or ex vivo) from the blood of the patient, for example, by cell sorting or affinity-based approaches, such as affinity chromatography or nanoparticles bearing immobilized epitope-tag specific antibodies (or alternative binding protein(s)) for capturing the CAR expressing T cells. Respective materials and methods are known and available in the art. Moreover, the incorporation of an epitope-tag in the ectodomain of the CAR can also enable depletion of the modified T cell(s) through administering a tag-specific antibody (preferably a monoclonal antibody, or alternative binding protein) to the patient. This will enable (partial or complete) elimination of the CAR T cells from the patient's body, for example, in cases where the therapeutic action of the CAR T cells is no longer needed or required to be diminished (e.g., in the unlikely case of an unintended adverse event), thus providing a safety means (“safety switch”) for interfering with and controlling the therapy.

One commonly used epitope-tag is a Myc-tag (EQKLISEEDL; SEQ ID NO: 7), which is a peptide derived from the c-Myc protein. Myc-tag specific antibodies are commercially available (e.g., c-Myc monoclonal antibody from Invitrogen). However, in view of c-Myc protein being a gene product of the human c-myc proto-oncogene, the presence of a Myc-tag in a CAR may, in certain conceivable instances, be less favorable, for example, where its presence could provide a hindrance (or even an exclusion criterion) for regulatory approval of clinical applications of respective CD28 CAR T cells. Thus, in cases where the inclusion of an epitope-tag is envisaged, it is preferred that an epitope-tag will be employed that is distinct from a Myc-tag and unrelated to c-Myc protein or any other gene product derived from any other (proto-)oncogene. For analogous reasons, it is understood that preferably an epitope-tag is selected that is distinct from any (poly)peptide derived from known human pathogens (such as virus or other microbial pathogens) that may bear a risk for (i) giving rise to false-positive detection of such a pathogen when assaying the blood of a recipient of respective CAR T cells, and/or (ii) triggering undesired immune responses against the CAR or CAR-expressing modified T cell, of which either possibility may provide a bar for regulatory approval of the CAR T cells. The skilled person will be able to select suitable epitope-tag(s) which circumvent such issues.

One exemplary, particularly preferred epitope-tag for being incorporated in the CARs of the present invention is the truncated form of the EGF receptor (EGFRt) as described in Ref. 30. The EGFRt epitope-tag is derived from the human full-length EGFR and truncated to ensure non-functionality for physiological EGFR activities.

It is understood that in cases where an epitope-tag is comprised in the ectodomain of the CAR, the epitope-tag is preferably comprised C-terminal of the (at least one) antigen binding moiety (e.g., the CD28-binding scFv) to not interfere with the binding of said antigen binding moiety to the targeted antigen. The skilled artisan is aware that, in embodiments where the epitope-tag is comprised in the ectodomain of the CAR, C-terminal of the (at least one) antigen binding moiety, the epitope-tag will be expressed as part of the CAR molecule (and thus does not require a further signal peptide).

Thus, in particularly preferred embodiments, the CAR comprises an EGFRt epitope-tag (without signal peptide) that is C-terminal of the antigen binding moiety, wherein preferably a hinge (or linker) region is between the antigen binding moiety and the EGFRt epitope-tag. Preferred hinge (or linker) regions are defined herein above and elsewhere in the specification.

There might also be instances where an epitope-tag within the ectodomain of a CAR may not be sterically accessible for an epitope-tag-specific antibody (or alternative binding protein) in order to enable a detection of the CAR molecule, e.g., due to the specific 3D-structural peculiarities of the individual CAR construct or other molecules on the surface of the modified T cell. Thus, it is also expressly contemplated herein that, in alternative embodiments of the modified T cell, the polynucleotide encoding a chimeric antigen receptor (CAR) in addition comprises a polynucleotide encoding an epitope-tag, wherein the polynucleotide encoding the CAR and the polynucleotide encoding the epitope-tag are configured to be expressed as separate (poly)peptide chains on the T cell surface. Strategies for multigene co-expression, including means and methods for construction of respective poly-/bi-cistronic expression constructs, are known and available in the art; see for example, Ref. 29. Moreover, exemplary means and methods employed in the art for co-expression of multiple genes from a single polynucleotide are based on the use of multiple promoters in a single vector, proteolytic cleavage sites between genes, internal ribosome entry sites, and so-called “self-cleaving” 2A peptides as described in Ref. 29.

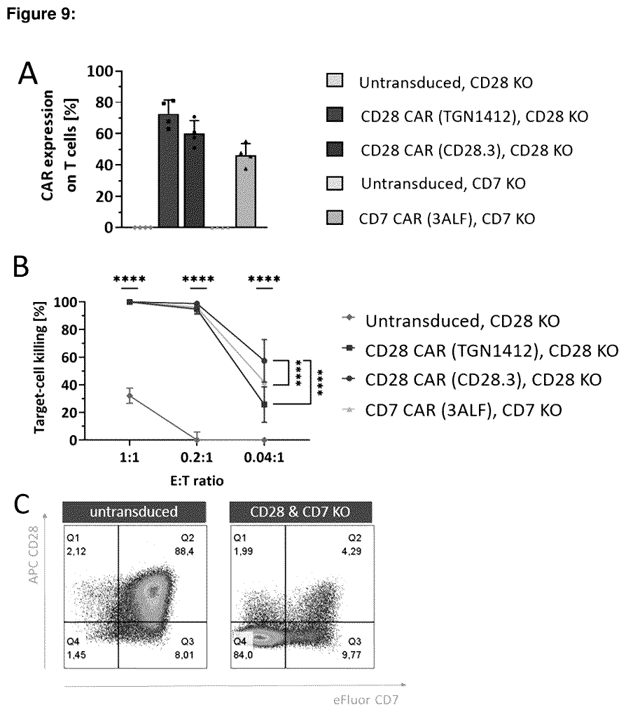

One exemplary, particularly preferred epitope-tag suitable for the latter embodiments is the truncated form of the EGF receptor (EGFRt) as described in Ref. 30 and exemplified herein in Example 5 (see also FIG. 8A). The EGFRt epitope-tag is derived from the human full-length EGFR and truncated to ensure non-functionality for physiological EGFR activities. The EGFRt epitope tag (including the signal peptide (indicated by underline)) comprises or consists of the amino acid sequence (SEQ ID NO: 55):

LLLVTSLLLCELPHPAFLLIPRKVCNGIGIGEFKDSLSINATNIKHFKN
CTSISGDLHILPVAFRGDSFTHTPPLDPQELDILKTVKEITGFLLIQAW
PENRTDLHAFENLEIIRGRTKQHGQFSLAVVSLNITSLGLRSLKEISDG
DVIISGNKNLCYANTINWKKLFGTSGQKTKIISNRGENSCKATGQVCHA
LCSPEGCWGPEPRDCVSCRNVSRGRECVDKCNLLEGEPREFVENSECIQ
CHPECLPQAMNITCTGRGPDNCIQCAHYIDGPHCVKTCPAGVMGENNTL
VWKYADAGHVCHLCHPNCTYGCTGPGLEGCPTNGPKIPSIATGMVGALL
LLLVVALGIGLFM.

Preferably, the polynucleotide sequence region encoding the CAR and the polynucleotide sequence region encoding the epitope-tag (e.g., the EGFRt epitope-tag) are comprised within the same expression cassette and interspaced by a polynucleotide sequence region which facilitates the expression of the CAR and the epitope-tag as separate (poly)peptides on the T cell surface. In a particularly preferred embodiment, these coding regions (i.e., encoding the CAR and the epitope-tag) are interspaced by a polynucleotide sequence encoding a “self-cleaving” 2A peptide (as described in Refs. 29,30), such as a “T2A linker peptide” (GSGEGRGSLLTCGDVEENPGP (SEQ ID NO: 56)) as described in Refs. 29,30 in used herein in Example 5; see also FIG. 8A.

Preferably only such (poly)peptides are selected to be comprised in the CAR, particularly in the ectodomain of the CAR, which have low (ideally no) immunogenicity and/or which do not comprise an Fc receptor binding epitope. Methods and assays for accessing the presence or absence of these properties are known in the art. Moreover, in order to allow an unambiguous detection of the epitope-tag, the epitope-tag is preferably a (poly)peptide that is typically/naturally absent from (or not expressed on) T-cell surfaces.

A CAR typically comprises a “transmembrane domain” (also interchangeably referred to herein as “transmembrane region”). The transmembrane domain links the extracellular portion of the CAR to the intracellular portion of the CAR and anchors the CAR to the cell membrane. Suitable transmembrane domains have the ability to be expressed at the surface of a T cell, and to connect the ectodomain comprising the antigen binding moiety with the intracellular signaling domain(s) for directing cellular response of the T cell against a defined target cell. The transmembrane domain is preferably a single-pass transmembrane domain, e.g., a single α-helix, that spans the membrane of the T cell. Alternatively, the transmembrane domain can be a multi-pass transmembrane domain. The transmembrane domain can be derived from a natural or synthetic source. For example, a transmembrane domain can be derived from any known transmembrane protein or membrane-anchored protein. Alternatively, the transmembrane domain can be synthetically/rationally designed and may consist largely of hydrophobic amino acid residues, such as leucine, valine, isoleucine and/or glycine.

In certain cases, a CAR may comprise a transmembrane domain derived from a known transmembrane protein. In cases where the primary sequence of a transmembrane protein is known, but it is unknown which exact portion of the transmembrane protein is the membrane-spanning region, one can use one of the multiple known prediction algorithms and/or online tools (for example, the “TMPred” tool as available on https://www.expasy.org/resources/tmpred; see also K. Hofmann & W. Stoffel (1993); Biol. Chem. Hoppe-Seyler 374,166) to reliably predict the amino acid sequence of a transmembrane domain of a transmembrane protein, and the obtained sequence can then be incorporated as a transmembrane domain into a CAR.

The endodomain of the CAR is the intracellular (i.e., cytoplasmic) portion of the CAR and is generally considered the “functional” end of the receptor. After extracellular antigen recognition by the at least one antigen binding moiety comprised in the ectodomain of the CAR, the CARs cluster, and an activating signal is transmitted through the endodomain of the CAR to the interior of the cell. Generally, an endodomain comprises at least one signaling domain, wherein the signaling domain upon extracellular antigen ligation of the CAR provides the necessary intracellular signal to drive T cell activation.

Initial CAR designs, now termed “first-generation” CARs, incorporated a single intracellular domain of CD3-zeta (CD3ζ), a component of the T cell antigen receptor (TCR) complex. Corresponding CAR T cells were activated by and exhibited cytotoxicity against target cells expressing the CAR target but failed to proliferate well and to elicit long-term antitumor responses. Subsequent “second-generation” CAR designs, which additionally incorporated the intracellular signaling domain of CD28 or of other co-stimulatory receptors (e.g., 4-1BB), improved T cell proliferation, cytokine production, and antitumor efficacy. Further enhancement of CAR T cell activity was achieved by the incorporation of more than one co-stimulatory domain, alongside CD3, characteristic of the so-called “third-generation” CARs. Meanwhile, numerous “third-generation” CARs having combinations of a variety of different co-stimulatory signaling domains have been generated (see, for example, Review Weinkove R et al., Clinical & Translational Immunology (2019); 8:e1049).

Previous attempts to extend the concept of CAR-T cell-based therapy also to malignancies of the T-lineage suffered from the absence of known suitable T cell surface antigen structures to be targeted by a CAR. This is because targeting antigens that are not exclusively expressed on malignant T cells, but also on non-malignant T cells, leads to T cell aplasia, resulting in severe immunodeficiency. Targeting antigens also expressed on the CAR T cells themselves leads to CAR-T cell fratricide (self-targeting and elimination). Although there are few antigens that were found to be predominantly expressed on malignant T cells while being nearly absent from physiological T cells, these antigens appeared to have no known functional relevance in malignant T cells. Their dispensability is hence likely to trigger immune escape mechanisms which gives rise to malignant T cell variants no longer expressing said antigens on their cell surface.

Seeking to overcome these previous shortcomings, the present inventors set out to develop a novel strategy for CAR-T cell therapy by generating a modified T cell characterized by having (i) a CAR which specifically targets the co-stimulatory molecule CD28; and (ii) a disrupted endogenous CD28-encoding gene.

This combined knock-out and knock-in gene editing approach yielded modified CAR-T cells with unexpected activation and expansion capacities, as well as enhanced selectivity and cytotoxic potency toward CD28-expressing T cells, while being resistant to fratricide.

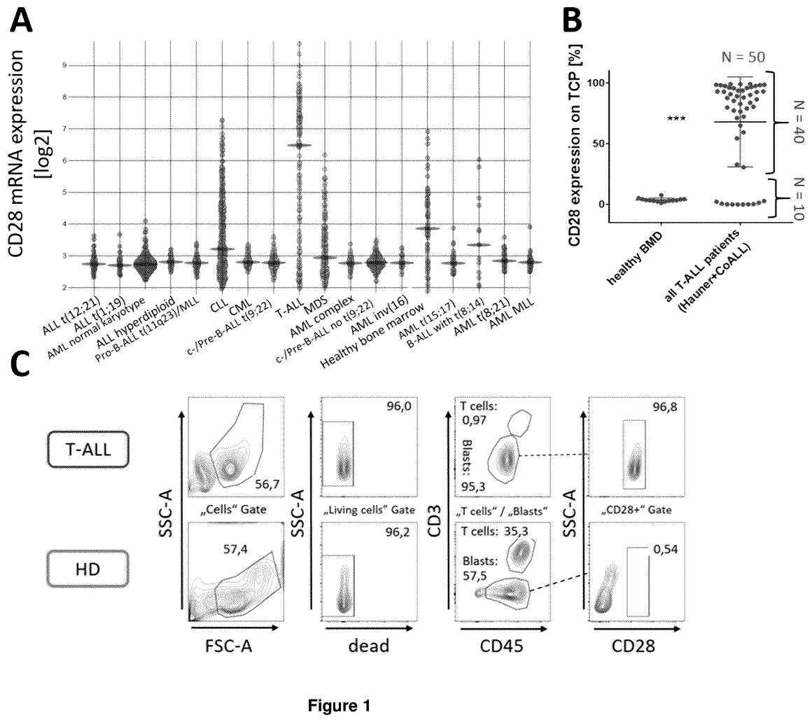

In view of CD28 being highly expressed on the majority of T-lineage malignancies, including T-lineage acute lymphoblastic leukemia and T-cell acute lymphoblastic leukemia (T-ALL) (FIG. 1), as well as in T cells implicated in the B-lineage malignancies, such as multiple myeloma (MM)¹³, the modified CD28-targeting CAR-T cells of the invention will provide unprecedented therapeutic means to selectively eradiate CD28-expressing T cells and associated T-cell-mediated effector functions (either being directly causative or indirectly contributing to disease pathology), particularly in the aforementioned malignancies.

Besides its role in malignancies of the T cell lineage, CD28 has also been found to being highly expressed on B cells in certain B cell malignancies, particularly in multiple myeloma (MM), and to also directly contribute to these disease pathologies. For example, CD28 was found to act as a key mediator of MM survival and proliferation, as well as to counteract apoptotic signaling possibly accounting for the frequently observed chemotherapeutic resistance (Bahlis N J et al., Blood. (2007); 109(11): 5002-5010; Murray M E et al. Blood. (2014); 123(24): 3770-3779). Accordingly, the modified T cells of the invention will also provide new therapeutic means for eradiating B cells in B cell malignancies, in particular in multiple myeloma (MM); and more generally, for the treatment of any other disorder characterized by the presence of CD28-expressing disease-promoting cells.

CD28 knockout in mice only leads to a mild immunodeficiency with immunoglobulin (IgG) levels reduced to ˜20% of CD28^{WT} mice, diminished IgG class switch upon viral infection while cytotoxic T cells could still be induced and were functional.¹⁴ Although CD28 was found to be expressed also on some physiological T cells, i.e., mature T cells and especially T_{Helper} cells, CD28 expression is lower in physiologic lymphoid precursor cells as compared to previously targeted T cell antigens, such as CD7. Depletion of CD28 is hence expected by the inventors to lead to a more specific, less generic immune compromise as compared to previous CAR T cell strategies targeting other surface antigens. Even in the unlikely case of side effects of anti-CD28 CAR T-cell therapy, the approach will still provide therapeutic benefit as a bridge-to-transplant solution. In addition, safety-switches can be incorporated into clinical-grade CD28-CAR designs, enabling on-demand CAR T-cell depletion in vivo.^{15, 16}

In a preferred embodiment of the modified T cell of the invention, the antigen binding moiety that is capable of specific binding to the extracellular portion of CD28 is an anti-CD28 antibody, preferably an anti-CD28 single-chain variable fragment (scFv); wherein preferably the anti-CD28 antibody or anti-CD28 scFv comprises: (a) a VH CDR1, CDR2 and CDR3 consisting of the amino acid sequences of SEQ ID NO: 8, 9 and 10, and a VL CDR1, CDR2 and CDR3 of the amino acid sequences of SEQ ID NO: 11, 12 and 13; or (b) a VH CDR1, CDR2 and CDR3 consisting of the amino acid sequences of SEQ ID NO: 14, 15 and 16, and a VL CDR1, CDR2 and CDR3 of the amino acid sequences of SEQ ID NO: 17, 18 and 19.

VH CDR1 of antibody TGN1412:
(SEQ ID NO: 8)
SYYIH;
VH CDR2 of antibody TGN1412:
(SEQ ID NO: 9)
CIYPGNVNTNYNEKFKD;
VH CDR3 of antibody TGN1412:
(SEQ ID NO: 10)
SHYGLDWNFDV;
VL CDR1 of antibody TGN1412:
(SEQ ID NO: 11)
HASQNIYVWLN;
VL CDR2 of antibody TGN1412:
(SEQ ID NO: 12)
KASNLHT;
VL CDR3 of antibody TGN1412:
(SEQ ID NO: 13)
QQGQTYPYT.
VH CDR1 of antibody CD28.3:
(SEQ ID NO: 14)
EYIIH;
VH CDR2 of antibody CD28.3:
(SEQ ID NO: 15)
WFYPGSNDIQYNAKFKG;
VH CDR3 of antibody CD28.3:
(SEQ ID NO: 16)
RDDFSGYDALPY;
VL CDR1 of antibody CD28.3:
(SEQ ID NO: 17)
RTNENIYSNLA;
VL CDR2 of antibody CD28.3:
(SEQ ID NO: 18)
AATHLVE;
VL CDR3 of antibody CD28.3:
(SEQ ID NO: 19)
QHFWGTPCT.

The amino acid sequences of the variable domains of monoclonal anti-human CD28 antibody “TGN1412” are available in published US patent: U.S. Pat. No. 8,709,414 B2. The amino acid sequence of the variable domains of the monoclonal anti-human CD28 antibody “CD28.3” are from GenBank entry AF451974.1 (https://www.ncbi.nlm.nih.gov/nuccore/AF451974; cf. Vanhove B et al. (2003) Blood; 102(2):564-70). The amino acid sequences of the complementarity-determining regions (CDRs) have been determined by analyzing the published antibody sequences using the abYsis webtool (http://www.abysis.org/; see also Swindells M B et al. (2017) J Mol Biol.; 429(3):356-364).

In a preferred embodiment of the modified T cell of the invention, the anti-CD28 antibody or anti-CD28 scFv comprises:
(a) a VH comprising or consisting of the amino acid sequence of SEQ ID NO: 20, and a VL comprising or consisting of the amino acid sequence of SEQ ID NO: 21; or
(b) a VH comprising or consisting of the amino acid sequence of SEQ ID NO: 22, and a VL comprising or consisting of the amino acid sequence of SEQ ID NO: 23; and/or
(c) wherein the anti-CD28 scFv has a linker between the C-terminus of the VL and the N-terminus of the VH, or between the C-terminus of the VH and the N-terminus of the VL;
   wherein preferably the anti-CD28 scFv has a linker comprising or consisting of the amino acid sequence of SEQ ID NO: 2; and/or
   wherein preferably the anti-CD28 scFv according to (a) has a VL-linker-VH configuration and/or the anti-CD28 scFv according to (b) has a VH-linker-VL configuration.

SEQ ID NO: 20 (VH of antibody TGN1412):
QVQLVQSGAEVKKPGASVKVSCKASGYTFTSYYIHWVRQAPGQGLEWIG
CIYPGNVNTNYNEKFKDRATLTVDTSISTAYMELSRLRSDDTAVYFCTR
SHYGLDWNFDVWGQGTTVTVSS.
SEQ ID NO: 21 (VL of antibody TGN1412):
DIQMTQSPSSLSASVGDRVTITCHASQNIYVWLNWYQQKPGKAPKLLIY
KASNLHTGVPSRFSGSGSGTDFTLTISSLQPEDFATYYCQQGQTYPYTF
GGGTKVEIKRTV.
SEQ ID NO: 22 (VH of antibody CD28.3):
VKLQQSGAELVKPGASVRLSCKASGYTFTEYIIHWIKLRSGQGLEWIGW
FYPGSNDIQYNAKFKGKATLTADKSSSTVYMELTGLTSEDSAVYFCARR
DDFSGYDALPYWGQGTMVTVSS.
SEQ ID NO: 23 (VL of antibody CD28.3):
DIQMTQSPASLSVSVGETVTITCRTNENIYSNLAWYQQKQGKSPQLLIY
AATHLVEGVPSRFSGSGSGTQYSLKITSLQSEDFGNYYCQHFWGTPCTF
GGGTKLEIKR.

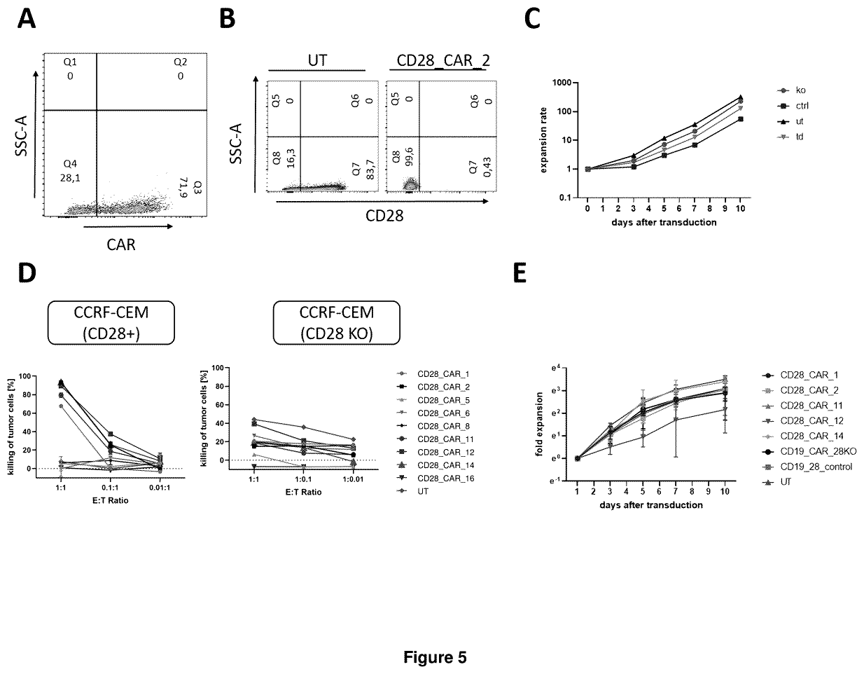

In an even more preferred embodiment, the anti-CD28 scFv comprises or consists of the amino acid sequence of SEQ ID NO: 24, SEQ ID NO: 25, or SEQ ID NO: 26 (corresponding to the scFvs of constructs CD28_CAR_2, CD28_CAR_12 and CD28_CAR_14 in Table 1). As observed by the inventors (see Example 2), constructs 2 and 14 (“CD28_CAR_2” and “CD28_CAR_14”) having a scFv defined by SEQ ID NO: 24 and SEQ ID NO: 26, respectively, performed best in terms of cytotoxic activity and expansion capacity (see FIG. 5). Thus, CARs comprising one or more of these two particular scFvs are particularly preferred. Moreover, as shown in Example 7, it was found by the inventors that CAR constructs bearing an anti-CD28 scFv as defined by SEQ ID NO: 24 performed best in terms of providing an enhancement of survival in a T-ALL mouse model. Thus, CARs comprising one or more scFv as defined by SEQ ID NO: 24 are particularly preferred.

SEQ ID NO: 24 (scFv of construct CD28_CAR_2
(without signal peptide)):
DIQMTQSPSSLSASVGDRVTITCHASQNIYVWLNWYQQKPGKAPKLLIY
KASNLHTGVPSRFSGSGSGTDFTLTISSLOPEDFATYYCQQGQTYPYTF
GGGTKVEIKRTVGSTSGSGKPGSGEGSTKGQVQLVQSGAEVKKPGASVK
VSCKASGYTFTSYYIHWVRQAPGQGLEWIGCIYPGNVNTNYNEKFKDRA
TLTVDTSISTAYMELSRLRSDDTAVYFCTRSHYGLDWNFDVWGQGTTVT
VSS.
SEQ ID NO: 25 (scFv of construct CD28_CAR_12
(without signal peptide)):
QVQLVQSGAEVKKPGASVKVSCKASGYTFTSYYIHWVRQAPGQGLEWIG
CIYPGNVNTNYNEKFKDRATLTVDTSISTAYMELSRLRSDDTAVYFCTR
SHYGLDWNFDVWGQGTTVTVSSGSTSGSGKPGSGEGSTKGDIQMTQSPS
SLSASVGDRVTITCHASQNIYVWLNWYQQKPGKAPKLLIYKASNLHTGV
PSRFSGSGSGTDFTLTISSLQPEDFATYYCQQGQTYPYTFGGGTKVEIK
RTV.
SEQ ID NO: 26 (scFv of construct CD28_CAR_14
(without signal peptide)):
VKLQQSGAELVKPGASVRLSCKASGYTFTEYIIHWIKLRSGQGLEWIGW
FYPGSNDIQYNAKFKGKATLTADKSSSTVYMELTGLTSEDSAVYFCARR
DDFSGYDALPYWGQGTMVTVSSGSTSGSGKPGSGEGSTKGDIQMTQSPA
SLSVSVGETVTITCRTNENIYSNLAWYQQKQGKSPQLLIYAATHLVEGV
PSRFSGSGSGTQYSLKITSLQSEDFGNYYCQHFWGTPCTFGGGTKLEIK
R.

In a further preferred embodiment of the modified T cell of the invention, the CAR further comprises an endodomain comprising one or more (more preferably two or more, even more preferably three or more) T-cell-stimulatory molecules; wherein the T-cell-stimulatory molecule is preferably a signaling domain from a T-cell-co-stimulatory receptor, an immunoreceptor tyrosine-based activation motif (ITAM), and/or a Toll/interleukin-1 receptor (TIR) domain; wherein preferably (i) the T-cell-co-stimulatory receptor is selected from: CD28, ICOS (CD278), CD27, 4-1BB (CD137, TNFRSF9), OX40 (CD134), IL-2Rβ, IL-15R-α, CD40L (CD154) and/or MyD88; and/or (ii) the ITAM is selected from: CD3-zeta (CD3ζ), DAP12, Fc-epsilon receptor 1 gamma chain, CD3-gamma, CD3-delta, CD3-epsilon, and CD79A (antigen receptor complex-associated protein alpha chain); and/or (iii) the TIR domain is the TIR domain of Toll-like receptor 2 (TLR2).

An “ITAM,” as used herein, is a conserved peptide motif that is found in the cytoplasmic portion of many signaling molecules expressed in many immune cells. The motif typically includes two repeats of the amino acid sequence YxxL/I (SEQ ID NO: 52) separated by 6-8 amino acids, wherein each X is independently any amino acid, producing the conserved motif YxxL/Ix₍₆₋₈₎YxxL/I (SEQ ID NO: 53). ITAMs within signaling molecules are important for signal transduction within the cell, which is mediated at least in part by phosphorylation of tyrosine residues in the ITAM following activation of the signaling molecule. ITAMs can also function as docking sites for other proteins involved in signaling pathways. In some embodiments, the endodomain of a CAR comprises at least 1, 2, 3, 4, or at least 5 ITAMs, wherein the ITAMs may be independently selected from the ITAMs comprised in CD3ζ, FcRγ and Megf10.

In another preferred embodiment of the modified T cell of the invention, the endodomain of the CAR comprises a CD28 signaling domain and a CD3-zeta (CD3ζ) signaling domain; wherein preferably the CD3-zeta (CD3ζ) signaling domain is carboxy(C)-terminal of the CD28 signaling domain.

For example, the amino acid sequence of the

human CD28 signaling domain is:
(SEQ ID NO: 27)
RSKRSRLLHSDYMNMTPRRPGPTRKHYQPYAPPRDFAAYRS;
murine CD28 signaling domain is:
(SEQ ID NO: 28)
NSRRNRLLQSDYMNMTPRRPGLTRKPYQPYAPARDFAAYRP;
rat CD28 signaling domain is:
(SEQ ID NO: 29)
NSRRNRLLQSDYMNMTPRRLGPTRKHYQPYAPARDFAAYRP.

For example, the amino acid sequence of the

human CD33 signaling domain is:
(SEQ ID NO: 30)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR;
murine CD3Z signaling domain is:
(SEQ ID NO: 31)
QSFGLLDPKLCYLLDGILFIYGVIITALYLRAKFSRSAETAANLQDPNQ
LYNELNLGRREEYDVLEKKRARDPEMGGKQQRRRNPQEGVYNALOKDKM
AEAYSEIGTKGERRRGKGHDGLYQGLSTATKDTYDALHMQTLAPR;
rat CD33 signaling domain is:
(SEQ ID NO: 32)
QSFGLLDPKLCYMLDGILFIYGVIVTALYLRAKFSRSADAAAYLQDPNQ
LYNELNLGRREEYDVLDKKRPRDPEMGGKQQRRRNPQEGVYNALQKDKM
AEAYSEIGMKGERRRGKGHDGLYQGLSTATKDTYDALHMQTLPPR.

In another preferred embodiment of the modified T cell of the invention, the endodomain of the CAR comprises a 4-1BB signaling domain and a CD3-zeta (CD3ζ) signaling domain; wherein preferably the CD3-zeta (CD3ζ) signaling domain is carboxy(C)-terminal of the 4-1BB signaling domain. Respective constructs are employed in Example 7.

For example, the amino acid sequence of the

human 4-1BB signaling domain is:
(SEQ ID NO: 59)
KRGRKKLLYIFKQPFMRPVQTTQEEDGCSCRFPEEEEGGCEL;
murine 4-1BB signaling domain is:
(SEQ ID NO: 60)
VLKWIRKKFPHIFKQPFKKTTGAAQEEDACSCRCPQEEEGGGGGYEL;
rat 4-1BB signaling domain is:
(SEQ ID NO: 61)
VLKNGTKEKDVVCGPPVVSLSPSTTPSAVTTPERESAFKKAVRTAQEED
ACSCRFPEEEEGGGGSYEL.

In another preferred embodiment of the modified T cell of the invention, the endodomain of the CAR comprises a CD28 signaling domain, a 4-1BB signaling domain, and a CD3-zeta (CD3ζ) signaling domain; wherein preferably the 4-1BB signaling domain is carboxy(C)-terminal of the CD28 signaling domain and/or the CD3-zeta (CD3ζ) signaling domain is carboxy(C)-terminal of the 4-1 BB signaling domain. Respective constructs are employed in Example 7.

In a further preferred embodiment of the modified T cell of the invention, the transmembrane domain comprises or consists of a transmembrane domain of a protein selected from the group of: a subunit of the T-cell receptor, CD3, CD4, CD7, CD8, CD27, CD28, OX40 (CD134), ICOS (CD278), PD-1 (CD279) and DAP12; more preferable from: CD3-zeta (CD3ζ), CD4, CD8, or CD28; even more preferable of a transmembrane domain of CD8, and most preferably of the transmembrane domain of CD8 alpha (CD8a).

For example, the amino acid sequence of the

human CD8 alpha transmembrane domain is:
(SEQ ID NO: 33)
IYIWAPLAGTCGVLLLSLVIT;
murine CD8 alpha transmembrane domain is:
(SEQ ID NO: 34)
IWAPLAGICVALLLSLIITLI;
rat CD8 alpha transmembrane domain is:
(SEQ ID NO: 35)
IWAPLAGICAVLLLSLVITLI.

It will be beneficial in certain cases to incorporate into a CAR a transmembrane domain which is extended by one or more consecutive amino acid residues of the intracellular portion of the natural transmembrane protein from which the transmembrane domain is derived. The additionally incorporated portion will then act as a hinge region between the transmembrane domain and the intracellular signaling domain of the CAR. Such a setup can improve signal transduction of a CAR.

For example, in cases where the transmembrane domain of a CAR is, or is derived from, the transmembrane domain of CD8 alpha (CD8a), the CAR may additionally comprise one or more consecutive amino acid residues of the intracellular portion of CD8 alpha (CD8a).

In preferred embodiments, the CAR comprises (i) the transmembrane domain of CD8 alpha (CD8a) and (ii) a peptide comprising the first seven amino acids of the intracellular portion of CD8 alpha (CD8a) (the latter peptide is referred to herein as “CD8 alpha intracellular peptide”).

For example, the amino acid sequence of the

human CD8 alpha intracellular peptide is:
(SEQ ID NO: 36)
LYCNHRN;
murine CD8 alpha intracellular peptide is:
(SEQ ID NO: 37)
CYHRSRK;
rat CD8 alpha intracellular peptide is:
(SEQ ID NO: 38)
CCHRNRR.

Accordingly, in an even more preferred embodiment, the CAR comprises a transmembrane domain and an endodomain, together comprising or consisting of the fused amino acid sequences of, in N- to C-terminal order, (i) a CD8 alpha transmembrane domain, (ii) a CD8 alpha intracellular peptide, (iii) a CD28 signaling domain, and (iv) a CD3 signaling domain. Thus, in cases where the T cell originates from human, the transmembrane domain and endodomain of the CAR, together, preferably comprise or consist of the amino acid sequence of: IYIWAPLAGTCGVLLLSLVITLYCNHRNRSKRSRLLHSDYMNMTPRRPGPTRKHYQPYAPPRDF AAYRSRVKFSRSADAPAYQQGQNQLYNELNLGRREEYDVLDKRRGRDPEMGGKPRRKNPQE GLYNELQKDKMAEAYSEIGMKGERRRGKGHDGLYQGLSTATKDTYDALHMQALPPR (SEQ ID NO: 39); corresponding to the fused amino acid sequences of, in N- to C-terminal order, (i) the human CD8 alpha transmembrane domain, (ii) the human CD8 alpha intracellular peptide, (iii) the human CD28 signaling domain, and (iv) the human CD3 signaling domain.

In an alternative preferred embodiment, the CAR comprises a transmembrane domain and an endodomain, together comprising or consisting of the fused amino acid sequences of, in N- to C-terminal order, (i) a CD8 alpha transmembrane domain, (ii) a CD8 alpha intracellular peptide, (iii) a 4-1BB signaling domain, and (iv) a CD3 signaling domain. Thus, in cases where the T cell originates from human, the transmembrane domain and endodomain of the CAR, together, preferably comprise or consist of the amino acid sequence of: IYIWAPLAGTCGVLLLSLVITLYCNHRNKRGRKKLLYIFKQPFMRPVQTTQEEDGCSCRFPEEEE GGCELRVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQALPPR (SEQ ID NO: 62); corresponding to the fused amino acid sequences of, in N- to C-terminal order, (i) the human CD8 alpha transmembrane domain, (ii) the human CD8 alpha intracellular peptide, (iii) the human 4-1 BB signaling domain, and (iv) the human CD3 signaling domain.

In further preferred embodiments, the CAR comprises a transmembrane domain and an endodomain, together comprising or consisting of the fused amino acid sequences of, in N- to C-terminal order, (i) a CD8 alpha transmembrane domain, (ii) a CD8 alpha intracellular peptide, (iii) a CD28 signaling domain, (iv) a 4-1 BB signaling domain, and (v) a CD3 signaling domain. Thus, in cases where the T cell originates from human, the transmembrane domain and endodomain of the CAR, together, preferably comprise or consist of the amino acid sequence of: IYIWAPLAGTCGVLLLSLVITLYCNHRNRSKRSRLLHSDYMNMTPRRPGPTRKHYQPYAPPRDF AAYRSKRGRKKLLYIFKQPFMRPVQTTQEEDGCSCRFPEEEEGGCELRVKFSRSADAPAYQQ GQNQLYNELNLGRREEYDVLDKRRGRDPEMGGKPRRKNPQEGLYNELQKDKMAEAYSEIGM KGERRRGKGHDGLYQGLSTATKDTYDALHMQALPPR (SEQ ID NO: 63); corresponding to the fused amino acid sequences of, in N- to C-terminal order, (i) the human CD8 alpha transmembrane domain, (ii) the human CD8 alpha intracellular peptide, (iii) the human CD28 signaling domain, and (iv) the human 4-1 BB signaling domain, and (v) the human CD3 signaling domain.

In a yet even further preferred embodiment, the CAR comprises, in N- to C-terminal order:
(a) an ectodomain comprising, or consisting of, the fused amino acid sequences of, in N- to C-terminal order: (i) a scFv capable of specific binding to the extracellular portion of CD28, preferably selected (or derived) from SEQ ID NO: 24, 25, and/or 26; and (ii) a hinge region, preferably selected (or derived) from SEQ ID NO: 5 and/or 6; the ectodomain optionally further comprising (iii) a (N-terminal) signal peptide (preferably SEQ ID NO: 4) and/or an epitope-tag (preferably SEQ ID NO: 7) C-terminally of the scFv;
and
(b) a transmembrane domain, preferably a CD8 alpha transmembrane domain (preferably SEQ ID NO: 33); optionally followed by a CD8 alpha intracellular peptide (preferably SEQ ID NO: 36);
and
(c) an endodomain comprising, or consisting of, the fused amino acid sequences of, in N- to C-terminal order:
   (c-1) (i) a CD28 signaling domain (preferably SEQ ID NO: 27); and (ii) a CD3 signaling domain (preferably SEQ ID NO: 30); or
   (c-2) (i) a 4-1BB signaling domain (preferably SEQ ID NO: 59); and (ii) a CD3 signaling domain (preferably SEQ ID NO: 30); or
   (c-3) (i) a CD28 signaling domain (preferably SEQ ID NO: 27); (ii) a 4-1 BB signaling domain (preferably SEQ ID NO: 59); and (iii) a CD3 signaling domain (preferably SEQ ID NO: 30).

Thus, in cases where the T cell originates from human, the CAR preferably comprises, or consists of, any one of the amino acid sequences defined by SEQ ID NOs: 40 to 44 and 64 to 67 (corresponding to the amino acid sequences of the constructs “CD28_CAR_1”, “CD28_CAR_2”, “CD28_CAR_11”, “CD28_CAR_12” and “CD28_CAR_14” of Example 2 and the constructs “CD28_CAR_21”, “CD28_CAR_22”, “CD28_CAR_23”, and “CD28_CAR_24” of Example 7, respectively), more preferably the CAR comprises, or consists of, SEQ ID NO: 41 or 44 (“CD28_CAR_2” and “CD28_CAR_14”, respectively, of Example 2) or any one of the amino acid sequences defined by SEQ ID NOs: 64 to 67 (corresponding to the amino acid sequences of the constructs “CD28_CAR_21”, “CD28_CAR_22”, “CD28_CAR_23”, and “CD28_CAR_24” of Example 7, respectively), most preferably the CAR comprises, or consists of, SEQ ID NO: 41, 64 or 66. Exemplary nucleotide sequences encoding the CARs defined by SEQ ID NOs: 40 to 44 and 64 to 67, respectively, are defined by SEQ ID NOs: 45 to 49 and 68 to 71, respectively.

SEQ ID NO: 40 (corresponding to construct CD28_CAR_1 including signal peptide):
MLLLVTSLLLCELPHPAFLLIPDIQMTQSPSSLSASVGDRVTITCHASQNIYVWLNWYQQKPGKA
PKLLIYKASNLHTGVPSRFSGSGSGTDFTLTISSLQPEDFATYYCQQGQTYPYTFGGGTKVEIK
RTVGSTSGSGKPGSGEGSTKGQVQLVQSGAEVKKPGASVKVSCKASGYTFTSYYIHWVRQA
PGQGLEWIGCIYPGNVNTNYNEKFKDRATLTVDTSISTAYMELSRLRSDDTAVYFCTRSHYGLD
WNFDVWGQGTTVTVSSEQKLISEEDLGGGGSGGGGSGGGGSIYIWAPLAGTCGVLLLSLVITL
YCNHRNRSKRSRLLHSDYMNMTPRRPGPTRKHYQPYAPPRDFAAYRSRVKFSRSADAPAYQ
QGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR.
SEQ ID NO: 41 (corresponding to construct CD28_CAR_2 including signal peptide):
MLLLVTSLLLCELPHPAFLLIPDIQMTQSPSSLSASVGDRVTITCHASQNIYVWLNWYQQKPGKA
PKLLIYKASNLHTGVPSRFSGSGSGTDFTLTISSLOPEDFATYYCQQGQTYPYTFGGGTKVEIK
RTVGSTSGSGKPGSGEGSTKGQVQLVQSGAEVKKPGASVKVSCKASGYTFTSYYIHWVRQA
PGQGLEWIGCIYPGNVNTNYNEKFKDRATLTVDTSISTAYMELSRLRSDDTAVYFCTRSHYGLD
WNFDVWGQGTTVTVSSEQKLISEEDLFVPVFLPAKPTTTPAPRPPTPAPTIASQPLSLRPEACR
PAAGGAVHTRGLDFACDIYIWAPLAGTCGVLLLSLVITLYCNHRNRSKRSRLLHSDYMNMTPRR
PGPTRKHYQPYAPPRDFAAYRSRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKDT
YDALHMQALPPR.
SEQ ID NO: 42 (corresponding to construct CD28_CAR_11 including signal peptide):
MLLLVTSLLLCELPHPAFLLIPQVQLVQSGAEVKKPGASVKVSCKASGYTFTSYYIHWVRQAPG
QGLEWIGCIYPGNVNTNYNEKFKDRATLTVDTSISTAYMELSRLRSDDTAVYFCTRSHYGLDW
NFDVWGQGTTVTVSSGSTSGSGKPGSGEGSTKGDIQMTQSPSSLSASVGDRVTITCHASQNI
YVWLNWYQQKPGKAPKLLIYKASNLHTGVPSRFSGSGSGTDFTLTISSLOPEDFATYYCQQGQ
TYPYTFGGGTKVEIKRTVEQKLISEEDLGGGGSGGGGSGGGGSIYIWAPLAGTCGVLLLSLVIT
LYCNHRNRSKRSRLLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR.
SEQ ID NO: 43 (corresponding to construct CD28_CAR_12 including signal peptide):
MLLLVTSLLLCELPHPAFLLIPQVQLVQSGAEVKKPGASVKVSCKASGYTFTSYYIHWVRQAPG
QGLEWIGCIYPGNVNTNYNEKFKDRATLTVDTSISTAYMELSRLRSDDTAVYFCTRSHYGLDW
NFDVWGQGTTVTVSSGSTSGSGKPGSGEGSTKGDIQMTQSPSSLSASVGDRVTITCHASQNI
YVWLNWYQQKPGKAPKLLIYKASNLHTGVPSRFSGSGSGTDFTLTISSLQPEDFATYYCQQGQ
TYPYTFGGGTKVEIKRTVEQKLISEEDLFVPVFLPAKPTTTPAPRPPTPAPTIASQPLSLRPEACR
PAAGGAVHTRGLDFACDIYIWAPLAGTCGVLLLSLVITLYCNHRNRSKRSRLLHSDYMNMTPRR
PGPTRKHYQPYAPPRDFAAYRSRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKDT
YDALHMQALPPR.
SEQ ID NO: 44 (corresponding to construct CD28_CAR_14 including signal peptide):
MLLLVTSLLLCELPHPAFLLIPVKLQQSGAELVKPGASVRLSCKASGYTFTEYIIHWIKLRSGQGL
EWIGWFYPGSNDIQYNAKFKGKATLTADKSSSTVYMELTGLTSEDSAVYFCARRDDFSGYDAL
PYWGQGTMVTVSSGSTSGSGKPGSGEGSTKGDIQMTQSPASLSVSVGETVTITCRTNENIYS
NLAWYQQKQGKSPQLLIYAATHLVEGVPSRFSGSGSGTQYSLKITSLQSEDFGNYYCQHFWG
TPCTFGGGTKLEIKREQKLISEEDLFVPVFLPAKPTTTPAPRPPTPAPTIASQPLSLRPEACRPAA
GGAVHTRGLDFACDIYIWAPLAGTCGVLLLSLVITLYCNHRNRSKRSRLLHSDYMNMTPRRPG
PTRKHYQPYAPPRDFAAYRSRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYD
ALHMQALPPR.
SEQ ID NO: 64 (corresponding to construct CD28_CAR_21 including signal peptide):
MLLLVTSLLLCELPHPAFLLIPDIQMTQSPSSLSASVGDRVTITCHASQNIYVWLNWYQQKPGKA
PKLLIYKASNLHTGVPSRFSGSGSGTDFTLTISSLQPEDFATYYCQQGQTYPYTFGGGTKVEIK
RTVGSTSGSGKPGSGEGSTKGQVQLVQSGAEVKKPGASVKVSCKASGYTFTSYYIHWVRQA
PGQGLEWIGCIYPGNVNTNYNEKFKDRATLTVDTSISTAYMELSRLRSDDTAVYFCTRSHYGLD
WNFDVWGQGTTVTVSSEQKLISEEDLFVPVFLPAKPTTTPAPRPPTPAPTIASQPLSLRPEACR
PAAGGAVHTRGLDFACDIYIWAPLAGTCGVLLLSLVITLYCNHRNKRGRKKLLYIFKQPFMRPV
QTTQEEDGCSCRFPEEEEGGCELRVKFSRSADAPAYQQGQNQLYNELNLGRREEYDVLDKR
RGRDPEMGGKPRRKNPQEGLYNELQKDKMAEAYSEIGMKGERRRGKGHDGLYQGLSTATKD
TYDALHMQALPPR
SEQ ID NO: 65 (corresponding to construct CD28_CAR_22 including signal peptide):
MLLLVTSLLLCELPHPAFLLIPVKLQQSGAELVKPGASVRLSCKASGYTFTEYIIHWIKLRSGQGL
EWIGWFYPGSNDIQYNAKFKGKATLTADKSSSTVYMELTGLTSEDSAVYFCARRDDFSGYDAL
PYWGQGTMVTVSSGSTSGSGKPGSGEGSTKGDIQMTQSPASLSVSVGETVTITCRTNENIYS
NLAWYQQKQGKSPQLLIYAATHLVEGVPSRFSGSGSGTQYSLKITSLQSEDFGNYYCQHFWG
TPCTFGGGTKLEIKREQKLISEEDLFVPVFLPAKPTTTPAPRPPTPAPTIASQPLSLRPEACRPAA
GGAVHTRGLDFACDIYIWAPLAGTCGVLLLSLVITLYCNHRNKRGRKKLLYIFKQPFMRPVQTT
QEEDGCSCRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYD
ALHMQALPPR
SEQ ID NO: 66 (corresponding to construct CD28_CAR_23 including signal peptide):
MLLLVTSLLLCELPHPAFLLIPDIQMTQSPSSLSASVGDRVTITCHASQNIYVWLNWYQQKPGKA
PKLLIYKASNLHTGVPSRFSGSGSGTDFTLTISSLQPEDFATYYCQQGQTYPYTFGGGTKVEIK
RTVGSTSGSGKPGSGEGSTKGQVQLVQSGAEVKKPGASVKVSCKASGYTFTSYYIHWVRQA
PGQGLEWIGCIYPGNVNTNYNEKFKDRATLTVDTSISTAYMELSRLRSDDTAVYFCTRSHYGLD
WNFDVWGQGTTVTVSSEQKLISEEDLFVPVFLPAKPTTTPAPRPPTPAPTIASQPLSLRPEACR
PAAGGAVHTRGLDFACDIYIWAPLAGTCGVLLLSLVITLYCNHRNRSKRSRLLHSDYMNMTPRR
PGPTRKHYQPYAPPRDFAAYRSKRGRKKLLYIFKQPFMRPVQTTQEEDGCSCRFPEEEEGGC
ELRVKFSRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
SEQ ID NO: 67 (corresponding to construct CD28_CAR_24 including signal peptide):
MLLLVTSLLLCELPHPAFLLIPVKLQQSGAELVKPGASVRLSCKASGYTFTEYIIHWIKLRSGQGL
EWIGWFYPGSNDIQYNAKFKGKATLTADKSSSTVYMELTGLTSEDSAVYFCARRDDFSGYDAL
PYWGQGTMVTVSSGSTSGSGKPGSGEGSTKGDIQMTQSPASLSVSVGETVTITCRTNENIYS
NLAWYQQKQGKSPQLLIYAATHLVEGVPSRFSGSGSGTQYSLKITSLQSEDFGNYYCQHFWG
TPCTFGGGTKLEIKREQKLISEEDLFVPVFLPAKPTTTPAPRPPTPAPTIASQPLSLRPEACRPAA
GGAVHTRGLDFACDIYIWAPLAGTCGVLLLSLVITLYCNHRNRSKRSRLLHSDYMNMTPRRPG
PTRKHYQPYAPPRDFAAYRSKRGRKKLLYIFKQPFMRPVQTTQEEDGCSCRFPEEEEGGCEL
RVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR

As a general note, it is contemplated herein that any embodiment disclosed herein and referring to a (poly)peptide (e.g., a (poly)peptide comprised in a CAR) defined by (or to being derived from) one or more particular amino acid sequences may alternatively also be implemented with a (poly)peptide only having, with increasing preference, at least 30%, at least 40%, at least 50%, at least 60%, at least 70%, at least 80%, at least 85%, at least 90%, at least 95%, at least 98%, or at least 99% sequence identity with the referred (poly)peptide having the one or more particular amino acid sequences. Means and methods for determining sequence identity are known in the art. Preferably, the BLAST (Basic Local Alignment Search Tool) program can be used.

Moreover, it is also contemplated that any of the herein defined optional components of a CAR or of the CAR-encoding polynucleotide may optionally be combined with any one or more of the other herein defined optional components of a CAR or of the CAR-encoding polynucleotide. For example, the CAR comprised in the modified T cell of the invention may comprise any one or more of the herein defined antigen binding moieties, signal peptides, epitope-tags, hinge regions, transmembrane domains and/or intracellular (signaling) domains and/or peptides.

The skilled person is aware that the risk of immunogenicity against a modified T cell (esp. when intended to be employed in an adoptive therapy setting) has to be minimized and that, accordingly, the original T cell itself which is subjected to the genetic engineering and each individual component of a CAR (or other exogenous (poly)peptide to be expressed by the modified T cell) is preferably selected in accordance with the kind of subject species for which the modified T cell is envisaged (i.e. the future recipient of a respective CAR T cell therapy). Thus, if the subject is a human (e.g., a patient suffering from a CD28-positive B- or T-lineage malignancy) or other particular mammalian species, the nucleotide sequence encoding each individual component of the CAR will preferably be selected to encode an amino acid sequence which corresponds to, or has at least high sequence identity with, the amino acid sequence of the respective (poly)peptide in human or other particular mammalian species. The term “high sequence identity” means, with increasing preference, at least 80%, at least 90%, at least 95%, or at least 98% sequence identity with the amino acid sequence of the respective (poly)peptide in human or the other particular mammalian species. Alternatively, or additionally, the immunogenicity of each (poly)peptide component can be assessed by (in vitro) assays. Respective assays and methods are known in the art.

It will be appreciated that the medical uses and therapeutic/preventive applications of the invention are primarily intended for application in human medicine but may equally be applied in veterinary medicine. Thus, it is understood that the term “subject”, “patient” or “individual”, in accordance with the medical uses or treatments of the invention, may be a mammal, preferably a human, or any other animal. For veterinary purposes, subjects include, for example, laboratory animals including mice, rats, rabbits, guinea pigs, hamsters, non-human primates, farm animals such as cows, sheep, pigs, horses, and goats, and poultry such as chickens, turkeys, ducks, and geese; companion animals such as dogs and cats; or exotic and/or zoo animals.

The term “gene editing”, also referred to as “genomic editing”, “gene engineering” or “genomic engineering”, is a type of genetic engineering in which nucleotide(s)/nucleic acid(s) is/are inserted, deleted, and/or substituted in a DNA sequence, such as in the genome of a target cell. Targeted gene editing enables insertion, deletion, and/or substitution at pre-selected sites in the genome of a target cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out (or knocked-down) due to the sequence alteration. Therefore, targeted gene editing may be used to disrupt endogenous gene expression. “Targeted integration” refers to a process involving insertion of one or more exogenous sequences, with or without deletion of an endogenous sequence at the insertion site.

Targeted gene editing can be achieved either through a nuclease-independent approach, or through a nuclease-dependent approach. In the nuclease-independent approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence. Alternatively, the nuclease-dependent approach can achieve targeted gene editing through the specific introduction of double strand breaks (DSBs) by specific rare-cutting nucleases (e.g., endonucleases). Such nuclease-dependent targeted gene editing utilizes DNA repair mechanisms, for example, non-homologous end joining (NHEJ), which occurs in response to DSBs. DNA repair by NHEJ often leads to random insertions or deletions (so-called “InDels”) of a small number of endogenous nucleotides. In contrast to NHEJ-mediated repair, repair can also occur by a homology-directed repair (HDR). When a transgene containing exogenous genetic material (e.g., a transgene comprising a polynucleotide encoding a CAR) flanked by a pair of homology arms is present, the exogenous genetic material can be introduced into the genome by HDR, which results in targeted integration of the exogenous genetic material.

Available endonucleases capable of introducing specific and targeted DSBs include, but are not limited to, zinc-finger nucleases (ZFN), transcription activator-like effector nucleases (TALEN), and RNA-guided CRISPR-Cas9 nuclease (CRISPR/Cas9; Clustered Regularly Interspaced Short Palindromic Repeats (CRISPR)/CRISPR-associated protein 9).

Zinc-finger nucleases (ZFNs) are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. The most recognized example of a ZFN is a fusion of the FokI nuclease with a zinc finger DNA binding domain.

A transcription activator-like effector nuclease (TALEN) is a targeted nuclease comprising a nuclease fused to a transcription activator-like (TAL) effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions termed repeat-variable di-residues (RVD). The most recognized example of a TALEN in the art is a fusion polypeptide of the FokI nuclease to a TAL effector DNA binding domain.

The CRISPR-Cas9 system is a naturally occurring defense mechanism in prokaryotes that has been repurposed as an RNA-guided DNA-targeting platform used for gene editing. It relies on the DNA nuclease Cas9 (CRISPR-associated protein 9), and two noncoding RNAs—crisprRNA (crRNA) and trans-activating crRNA (tracrRNA)—to target the cleavage of DNA. crRNA drives sequence recognition and specificity of the CRISPR-Cas9 complex through Watson-Crick base pairing typically with a 19 or 20 nucleotide (nt) sequence in the target DNA. Changing the nucleotide sequence of the first (5′) 19 or 20 nts (the so-called “spacer sequence”) in the crRNA (or alternatively sgRNA as mentioned below) allows targeting of the CRISPR-Cas9 complex to specific loci.

The CRISPR-Cas9 complex only binds such DNA sequences that (i) contain a target sequence (also known as “protospacer”) that matches with (i.e., is complementary to) the 19 or 20nt “spacer sequence” of the crRNA, and (ii) only if the target sequence (protospacer) is immediately followed by a specific short DNA motif (with the sequence NGG) referred to as a “protospacer-adjacent motif (PAM)” (on the strand opposite from the strand comprising the protospacer), in other words a PAM needs to be immediately downstream of the protospacer. TracrRNA hybridizes with the 3′-end of crRNA to form an RNA-duplex structure (also called “two-part guide RNA”) that is bound by the Cas9 endonuclease to form the catalytically active CRISPR-Cas9 complex, which can then cleave the target DNA. Alternatively, a “single guide RNA” (sgRNA) which refers to a synthetic fusion of a crRNA and a tracrRNA can be used. Design of crRNAs, tracrRNAs and sgRNAs are known in the art.

Once the CRISPR-Cas9 complex is bound to DNA at a target site, two independent nuclease domains within the Cas9 enzyme each cleave one of the DNA strands three nucleotides upstream of the PAM, leaving a double-strand break (DSB) where both strands of the DNA terminate in a base pair (a blunt end). After binding of CRISPR-Cas9 complex to DNA at a specific target site and formation of the site-specific DSB, the next step is repair of the DSB. Cells use two main DNA repair mechanisms to repair the DSB: non-homologous end joining (NHEJ) and homology-directed repair (HDR):

NHEJ is a robust repair mechanism that exists in the majority of cell types, including non-dividing cells. NHEJ is error-prone and typically results in the removal or addition of between one and several hundred nucleotides at the site of the DSB, though such modifications are typically <20 nt. The resulting insertions and deletions (‘InDels’) can disrupt coding or noncoding regions of genes.

HDR, on the other hand, uses a long stretch of homologous donor DNA, provided endogenously or exogenously, to repair the DSB with high fidelity. HDR is active only in dividing cells and occurs at a relatively low frequency in most cell types. In many embodiments of the present disclosure, NHEJ is utilized as the repair operant.

In some embodiments, the Cas9 endonuclease is from the bacterial species Streptococcus pyogenes, although other Cas9 homologs may be used. It should be understood, that wild-type Cas9 may be used, or modified versions of Cas9 may be used (e.g., evolved versions of Cas9, or Cas9 orthologues or variants) as known in the art. In some embodiments, Cas9 may be substituted with another RNA-guided endonuclease, such as Cpf1 (of a class II CRISPR/Cas system).

In a preferred embodiment of the modified T cell of the invention, the disrupted endogenous CD28-encoding gene comprises one or more mutations, i.e., one or more nucleotide base insertions and/or deletions (‘InDels’), e.g., introduced by means of targeted gene editing, by which the reading frame of the endogenous CD28-encoding gene is disrupted, thereby preventing the functional expression of the CD28 protein.

Methods for introducing such disrupting mutations into a target gene (i.e., the CD28-encoding gene) in living mammalian cells (such as T cells) are known in the art and are described herein. Respective methods particularly envisaged by the present invention, without being limiting, are nuclease-based gene editing methods including, with increasing preference, zinc finger nuclease (ZFN)-based gene editing, transcription activator-like effector nuclease (TALEN)-based gene editing, and/or Clustered Regularly Interspaced Short Palindromic Repeats (CRISPR)/Cas-based gene editing, as discussed, for example, in Gaj T. et al. (2013) Trends Biotechnol. 31(7): 397-405; Carroll D. (2012) Molecular therapy 20(9):1659-1660; Xiao A. et al. (2013) Nucleic Acids Research. 1-11. The application of these three nuclease-based gene-editing methods in the context of disrupting a target gene in T cells has been previously reported, for example, in Fleischer et al. (2019) J Hematol Oncol 12:141.

Although in certain preferred embodiments the polynucleotide encoding the CAR is integrated in the genome of the T cell, alternative embodiments are contemplated herein wherein the polynucleotide is not integrated in the genome of the T cell. For example, genomic integration may be preferred in cases where a long-lasting (or permanent) expression of the transgene (e.g., a CAR) is desired, non-integrative approaches may be favorable in cases where only a transient expression of the transgene would be beneficial.

A variety of methods for integrative and non-integrative gene transfer (or transgene delivery) into T cells is available in the art (see Review by Guedan S et al., Mol Ther Methods Clin Dev. 2018 Dec. 31; 12:145-156), and some of which are described herein in further detail. It is understood that each of these methods may optionally be employed for certain embodiments contemplated herein.

Methods for non-integrative gene transfer include RNA transfection, wherein a cell (e.g., a T cell) is transfected with an RNA encoding the transgene (e.g., the CAR), leading to a transient expression of the transgene (without integration into the genome) that will be rapidly diluted with ongoing expansion of the T cells (see Maude et al. (2015) Blood 125 (26): 4017-4023).

Methods for integrative gene transfer can be further divided into those resulting in a non-targeted integration of the transgene (i.e., insertion of the transgene at a random chromosomal position), such as retroviral and lentiviral vector-based methods; and those resulting in a targeted integration of the transgene (i.e., insertion at a particular chromosomal locus), e.g., endonuclease-based methods as referred to herein.

In certain instances, the polynucleotide encoding the CAR may additionally comprise an exogenous promoter in operable linkage with the CAR-coding nucleotide sequence to facilitate the functional expression of the CAR. In other instances, the polynucleotide encoding the CAR is integrated in the genome of a T cell in operable linkage with the endogenous promoter of an endogenous gene. In such instances, the CAR will be expressed under the control of said endogenous promoter, and the polynucleotide encoding the CAR is not needed to additionally comprise an exogenous promoter. In either case, the exogenous or endogenous promoter may be a constitutively active promoter, an inducible promoter, or a tissue-specific promoter.

In a particular embodiment, the polynucleotide encoding the CAR is integrated in the endogenous CD28-encoding gene, thereby disrupting the reading frame of the endogenous CD28-encoding gene.

In alternative embodiments, the polynucleotide encoding the CAR is integrated in-frame in the endogenous CD28-encoding gene (e.g., by homology-directed repair (HDR) or homologous recombination) and such that the portion of the endogenous CD28-encoding gene which encodes the extracellular region of CD28 (i.e., exon 2) is replaced by the polynucleotide encoding the CAR.

In this embodiment, the CAR will be expressed under the control of the endogenous promoter of the endogenous-CD28 encoding gene, and as a fusion protein with the transmembrane domain and intracellular domain of the endogenous CD28-encoding gene. In such embodiments, the resulting chimeric fusion protein optionally comprises one or more additional T cell-stimulatory domains (preferably, a CD3 signaling domain) in or C-terminally fused to, the intracellular domain of the endogenous CD28.

In accordance with a preferred embodiment of the first aspect of the invention, the disruption of the endogenous CD28-encoding gene is due to one or more nucleotide base insertions and/or deletions (‘InDels’) resulting from non-homologous end joining (NHEJ) DNA repair of DNA double-strand breaks (DSBs); wherein the DSBs are preferably resulting from a nuclease-based gene editing with a zinc finger nuclease (ZFN), transcription activator-like effector nuclease (TALEN), and/or Clustered Regularly Interspaced Short Palindromic Repeats (CRISPR)/Cas-based RNA-guided DNA endonuclease; and/or wherein the CAR-encoding polynucleotide is preferably integrated into the genome of the T cell, preferably by ex vivo retrovirus-based gene delivery.

In a preferred embodiment of the modified T cell of the invention, the CAR comprises in its ectodomain at least one further antigen binding moiety that binds specifically to a different (T-cell and/or B-cell) surface molecule than CD28;
wherein preferably said T-cell surface molecule is:
(i) selected from CD2, CD5, CD7, TRBC1, CD30, CD37, and/or CD1a; and/or
(ii) upregulated on malignant T cells; and/or
(iii) not or only to a limited extent expressed on normal T cells and/or on other hematopoietic cells;
and/or
wherein preferably said B-cell surface molecule is:
(i) selected from CD19 and/or CD22; and/or
(ii) upregulated on malignant B cells; and/or
(iii) not or only to a limited extent expressed on normal B cells and/or on other hematopoietic cells.

In a particularly preferred embodiment, the CAR comprises in its ectodomain at least one further antigen binding moiety that binds specifically to a different surface molecule than CD28, wherein said further surface molecule is CD7.

In preferred embodiments of the latter embodiment, the antigen binding moiety that binds specifically to CD7 is an anti-CD7 antibody, preferably an anti-CD7 single-chain variable fragment (scFv); wherein preferably the anti-CD7 antibody or anti-CD7 scFv comprises (a) a VH comprising or consisting of the amino acid sequence of SEQ ID NO: 57, and a VL comprising or consisting of the amino acid sequence of SEQ ID NO: 58; wherein preferably the VH and VL are configured in a VH-linker-VL configuration, the linker having an amino acid sequence comprising or consisting of SEQ ID NO: 2.

SEQ ID NO: 57 (VH of a preferred anti-CD7 scFv):
QVQLQESGAELVKPGASVKLSCKASGYTFTSYWMHWVKQRPGQGLEWIG
KINPSNGRTNYNEKFKSKATLTVDKSSSTAYMQLSSLTSEDSAVYYCAR
GGVYYDLYYYALDYWGQGTTVTVSS
SEQ ID NO: 58 (VL of a preferred anti-CD7 scFV):
DIELTQSPATLSVTPGDSVSLSCRASQSISNNLHWYQQKSHESPRLLIK
SASQSISGIPSRFSGSGSGTDFTLSINSVETEDFGMYFCQQSNSWPYTF
GGGTKLEIKR

In alternative embodiments, the anti-CD7 antibody or anti-CD7 single-chain variable fragment (scFv) comprises the CDRs as comprised in the VH defined by SEQ ID NO: 57 and the VL defined by SEQ ID NO: 58.

In a preferred embodiment of the modified T cell of the invention, the modified T cell further comprises one or more polynucleotides encoding one or more additional CARs, wherein the one or more additional CARs comprise in their ectodomain at least one antigen binding moiety that is capable of specific binding to a different (T-cell and/or B-cell) surface molecule than CD28; wherein preferably said T-cell surface molecule is:
(i) selected from CD2, CD5, CD7, TRBC1, CD30, CD37, and/or CD1a; and/or
(ii) upregulated on malignant T cells; and/or
(iii) not, or only to a limited extent, expressed on normal T cells and/or other hematopoietic cells;
and/or
wherein preferably said B-cell surface molecule is:
(i) selected from CD19 and/or CD22; and/or
(ii) upregulated on malignant B cells; and/or
(iii) not or only to a limited extent expressed on normal B cells and/or on other hematopoietic cells.

It is understood that in embodiments wherein the additionally targeted antigen is a T-cell surface molecule, the modified T cell may additionally have the respective endogenous T-cell surface molecule-encoding gene disrupted, in order to prevent self-targeting (and fratricide) of the modified T cells. The skilled person will be able to effecting a respective additional endogenous gene disruption by employing analogous means and methods as described herein for effecting the disruption of the endogenous CD28-encoding gene.

In particularly preferred embodiments, the modified T cell further comprises one or more polynucleotides encoding one or more additional CARs, wherein the one or more additional CARs comprise in their ectodomain at least one antigen binding moiety that is capable of specific binding to a different (T-cell and/or B-cell) surface molecule than CD28; wherein said surface molecule is CD7.

In preferred embodiments, the antigen binding moiety that binds specifically to CD7 is an anti-CD7 antibody, preferably an anti-CD7 single-chain variable fragment (scFv); wherein preferably the anti-CD7 antibody or anti-CD7 scFv comprises (a) a VH comprising or consisting of the amino acid sequence of SEQ ID NO: 57, and a VL comprising or consisting of the amino acid sequence of SEQ ID NO: 58. In alternative embodiments, the anti-CD7 antibody or anti-CD7 single-chain variable fragment (scFv) comprises the CDRs as comprised in the VH defined by SEQ ID NO: 57 and the VL defined by SEQ ID NO: 58.

The skilled person understands in view of the present disclosure that a respective additional CAR (e.g., a CAR directed against CD7) may comprise analogous further components (with exception of a CD28-binding moiety) as described herein for the CAR comprising a CD28-binding moiety.

An exemplary CD7-directed CAR, which may be employed for the herein disclosed embodiments, is described in Ref. 12.

In another preferred embodiment of the modified T cell of the invention, the modified T cell further comprises a “suicide system” (commonly also referred to as “safety switch”). Suicide systems provide a mechanism whereby the modified T cell can be deactivated or destroyed. Such a feature allows precise therapeutic control of any treatments wherein the modified T cells are used. Suicide systems provide a means to deactivate CAR T cells if and when either cytokine-mediated or on-target, off-tumor toxicities occur. Numerous suicide systems are known in the art: for example, a corresponding T cell may be engineered to co-express the CAR and cell surface antigens for which FDA-approved therapeutic antibodies already exist. In case of a CAR-T cell mediated toxicity, the CAR-T cells can be eliminated by administration of corresponding antibodies and resulting cytotoxic mechanisms. Another form of “safety switch” is based on the expression of an apoptosis-triggering fusion protein comprising caspase 9 linked to a modified form of the FK506-binding protein FKBP1A (iCasp9), with the latter enabling conditional dimerization and activation of the fusion protein through binding to a systemically administered and otherwise biologically inert small molecule (AP1903). Various safety switches are known in the art (e.g., discussed in Review: “Engineering strategies to overcome the current roadblocks in CAR T cell therapy. Rafiq S et al., Nat Rev Clin Oncol. (2020); 17(3):147-167), and optional incorporation of any of these in the modified T cell of the invention is contemplated.

In a second aspect, the invention relates to a population of modified T cells comprising the modified T cell of the invention, wherein (a) at least 25%, preferably at least 50%, or more preferably at least 70% of the modified T cells of the population express the CAR on their surface; (b) at least 25%, preferably at least 50%, or more preferably at least 70% of the modified T cells of the population express the CAR following at least 5 days, preferably at least 7 days, or more preferably at least 10 days of in vitro proliferation; and/or (c) at least 50%, preferably at least 60%, more preferably at least 70%, even more preferably at least 80%, or even more preferably at least 90% of the modified T cells of the population do not express a detectable level of CD28 protein; and/or (d) the population, when co-cultured in vitro with a population of non-modified T cells that express CD28, induces cell lysis of at least 40%, preferably at least 50%, or more preferably at least 60% of the non-modified T cells in the culture, wherein the initial ratio of modified to non-modified T cells is about equal; and/or (e) the modified T cells in the population have an in vitro clonal expansion rate of at least 30% per day.

The term “population of modified T cells” as used herein refers to more than one modified T cell of the invention.

Methods for qualitatively and/or quantitatively evaluating the surface expression of a cell surface marker of interest (e.g., CD28), e.g., for assessing the knock-out efficiency of a gene that has been subjected to a targeted gene editing, are known in the art, including, without limitation, flow cytometry (including fluorescence-activated cell sorting (FACS)) and/or Enzyme-linked Immunosorbent Assay (ELISA). Alternatively, the CD28-KO in the modified T cells of the invention may also be assessed on the genetic level by detecting the presence or absence of mutations in the endogenous CD28-encoding gene in the obtained T cells, for example, without limitation, via DNA sequencing (e.g., Sanger sequencing) at the genetic locus (target site) of the crRNA and optionally via TIDE analysis (Brinkman E K et al. Nucleic Acids Res. (2014); 42(22): e168). Methods for determining the in vitro clonal expansion rate (i.e. the percentage increase in cell number relative to a starting cell number measured over a given time period (at standard culture conditions (37° C.)) of a T cell or a population of T cells, such as determining cell counts by using a hemocytometer, are known in the art.

In a third aspect, the invention relates to a method for generating modified T cells in vitro, comprising (a) disrupting the endogenous CD28-encoding gene in T cells; and (b) introducing into said T cells a polynucleotide encoding a chimeric antigen receptor (CAR), wherein the CAR comprises in its ectodomain at least one antigen binding moiety that is capable of specific binding to the extracellular portion of CD28.

The T cells as used in step (a) of the method of the invention may originate from any source known in the art. For example, T cells can be differentiated in vitro from a hematopoietic stem cell population, or T cells may be obtained from a donor (i.e., the donor's body). The donor may be autologous (e.g., the prospective recipient of the modified T cells of the invention) or allogeneic relative to the recipient. In cases where the modified T cells are envisaged for a recipient suffering from a T-cell malignancy, the T cells originate preferably from an allogeneic healthy donor (or any alternative source, other than the envisaged recipient). In this case, the donor is preferably at least partially human leukocyte antigen (HLA)-matched with the prospective recipient of the T cells. Yet, in other cases where the recipient of the modified T cells has a disorder, wherein the T cells are not malignant but merely contribute to disease progression, e.g., through their interaction with malignant cells of other types (e.g., malignant B cells in multiple myeloma (MM)), the T cells may originate from the recipient itself (autologous). In a preferred embodiment the donor is a mammal, preferably a human.

T cells can be isolated from, e.g., peripheral blood mononuclear cells (PBMCs), bone marrow, lymph node tissue, cord blood, thymus tissue, tissue from a site of infection, ascites, pleural effusion, spleen tissue, and tumors. The term “peripheral blood mononuclear cell” (PBMC), refers to a population of white blood cells having a round nucleus, i.e., including lymphocytes (T cells, B cells and NK cells) and monocytes, which has not been enriched for a given sub-population. PBMCs may be obtained from whole blood samples by any suitable method known in the art. By way of example, PBMCs can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer, which will preferentially lyse red blood cells. For the clinical setting, PBMCs can also be isolated from a donor's whole blood by leukapheresis. In addition, the T cells can be derived from one or more T cell lines available in the art. T cells can also be obtained from an artificial thymic organoid (ATO) cell culture system, which replicates the human thymic environment to support efficient ex vivo differentiation of T-cells from primary and reprogrammed pluripotent stem cells. Additional methods of isolating T cells are known in the art.

In a preferred embodiment of the method of the invention, the method further comprises, prior to (a), one or more of the following steps:
(0a-i) isolating T cells from a peripheral blood mononuclear cell (PBMC) sample of a donor; and/or
(0a-ii) enriching the T cells for one or more T-cell sub-populations based on surface marker expression and/or antigen-binding specificity; and/or
(0a-iii) activating the T cells by contacting the T cells in vitro with one or more T-cell-activating agents; and optionally removing the one or more T-cell-activating agents after activation.

In a preferred embodiment, the T cells used in step (a) are CD3⁺ T cells; wherein the CD3⁺ T cells are preferably isolated from a peripheral blood mononuclear cell (PBMC) sample obtained from a healthy donor or a patient suffering from a CD28-positive malignancy.

Preferable T cell sub-populations that may be enriched in (optionally additional) step (0a-ii) are CD4⁺ T cells and/or CD8⁺ T cells. For example, it is particular envisaged herein that modified T cells are generated from selectively enriched CD4⁺ T cells, and in parallel, yet, in a separate batch, from selectively enriched CD8⁺ T cells, wherein both, the CD4⁺ T cells and the CD8⁺ T cells, originate from the same donor. A corresponding setup will allow to obtain a modified T cell product which comprises a pre-defined (and adaptable) ratio of modified (CD4⁺) T cells and modified (CD8⁺) T cells. This will allow to adapt and to balance T cell helper and effector functions to the individual needs of each individual patient, i.e. in a personalized manner, thereby providing a further level of safety, reduced risk of side effects, and enhanced efficacy.

Means and methods for isolating and/or enriching of T cells or sub-populations of T cells based on the presence or absence of certain surface molecules (e.g., CD3) are known in the art.

In another (or even more) preferred embodiment, the T cells used in step (a) are activated T cells.

T cell activation is characterized by an upregulation of certain surface markers, the expression of pro-inflammatory cytokines and/or proliferation activity. Any of these parameters can be used for assessing the activation status of T cells. Exemplary T cell activation markers and pro-inflammatory cytokines whose expression and/or secretion may be assessed are, without being limiting, CD25 (IL2RA), CD30, CD38, CD69, CD95 (FASR), CD137 (4-1BB), CD154 (CD40L) and IL-2, IL-4, IL-5, IL-6, IL-9, IL-13, IL-17, TNFα, IFNγ, respectively. Agents suitable for inducing T-cell activation, referred to herein as “T-cell-activating agents”, may be one or more agonists of a T-cell activating receptor, preferably one or more CD3 agonists and/or CD28 agonists; more preferable one or more anti-CD3 agonistic antibodies and/or anti-CD28 agonistic antibodies. In a preferred embodiment, the T-cell-activating agents are anti-CD3 and anti-CD28 agonistic antibodies; most preferably the T-cell-activating agents are anti-CD3 and anti-CD28 agonistic antibodies conjugated on (magnetic) beads or a matrix, wherein the beads or matrix enables separation/removal of the T-cell-activating agents from the T cells. For example, magnetic beads (such as Gibco™ Dynabeads™ Human T-Activator CD3/CD28 from ThermoFisher Scientific) can be removed by centrifugation and/or magnetic separation techniques as known in the art. An exemplary matrix-based T cell activating agent, as has proven particularly effective in the present case, is the reagent T Cell TransAct (Miltenyi Biotec) which is a polymeric nanomatrix conjugated to humanized recombinant CD3 and CD28 agonists. Other T-cell-activating agents are known in the art (e.g., Interferon-gamma (IFNγ), interleukins (such as interleukin (IL)-2, -7, -15 and/or -21), artificial antigen presenting cells, PMA/ionomycin, toxins (e.g., staphylococcal enterotoxin B (SEB)), or the non-toxic alternative CytoStim™) and may also be used.

It is also contemplated herein that several (e.g., two, three, four, or more) different T-cell activating agents can be used in combination (simultaneously or sequentially). For example, in the present case, T cell activation was particularly effective when conducted by CD3/CD28 co-stimulation (e.g., by using T Cell Transact (Miltenyi Biotec)) in combination (simultaneously) with interleukins (e.g., IL-7 and IL-15). Corresponding combinations are hence preferably employed in the method of the invention.

In a preferred embodiment of the method of the invention, step (a) is conducted within a time period of between 0 hours and 96 hours, between 6 hours and 72 hours, between 12 hours and 60 hours, between 18 hours and 54 hours, or between 24 hours and 48 hours,
(i) after the T cells were obtained from the donor's body; and/or
(ii) after the initiation of the activation in step (0a-iii).

The term “initiation of the activation”, as used herein, refers to the time point of contacting the T cells with the one or more T-cell activating agents.

In another preferred embodiment of the method of the invention, step (a) is conducted at a time point of about 48 hours (+/−12 hours), more preferably at a time point of about 48 hours (+/−6 hours) after the initiation of the activation in step (0a-iii).

In a preferred embodiment of the method of the invention, the disruption of the endogenous CD28-encoding gene is conducted by introducing one or more nucleotide base insertions and/or deletions (‘InDels’) resulting from non-homologous end joining (NHEJ) DNA repair of DNA double-strand breaks (DSBs); wherein the DSBs are preferably effected by applying a nuclease-based gene editing, preferably a zinc finger nuclease (ZFN)-based gene editing, a transcription activator-like effector nuclease (TALEN)-based gene editing, and/or a Clustered Regularly Interspaced Short Palindromic Repeats (CRISPR)/CRISPR-associated protein 9 (Cas9)-based gene editing.

In a preferred embodiment of the method of the invention, step (a) comprises: (a-i) introducing a CRISPR/Cas9 system into the T cells, wherein the CRISPR/Cas9 system comprises:
(i) a Cas9 protein, or a nucleic acid molecule encoding a Cas9 protein; and
(ii) a crisprRNA (crRNA), or a nucleic acid molecule encoding a crRNA, wherein the crRNA comprises a spacer sequence which specifically targets the endogenous CD28-encoding gene; and
(iii) a tracrRNA, or a nucleic acid molecule encoding a tracrRNA, wherein the tracrRNA forms a complex with the Cas9 protein and the crRNA.

In a particularly preferred embodiment, the CRISPR/Cas9 system is introduced as a RNP complex, wherein the RNP complex comprises a crRNA, a tracrRNA and Cas9 nuclease; wherein the CRISPR/Cas9 system is preferably introduced into the T cells by means of electroporation, preferably in the presence of an electroporation enhancer. Suitable electroporation methods and conditions are known in the art as described herein; and suitable electroporation devices and enhancers (e.g., Alt-R® Cas9 Electroporation Enhancer (IDT); see Example 2) are commercially available.

It is understood that the crRNA and tracrRNA may be provided as two individual molecules (i.e., as two guide RNAs (gRNAs) or pre-assembled as “two-part guide RNA”); or can alternatively be provided as a “single guide RNA (sgRNA)”.

In certain embodiments, the CRISPR/Cas9 system includes multiple gRNAs (two-part guide RNAs or sgRNAs). These guide RNAs may target a single gene (e.g., the endogenous CD28-encoding gene) at multiple nucleotide positions, or they may target multiple genes of interest.

In a preferred embodiment, the spacer sequence targets the endogenous CD28-encoding gene within a portion (preferably an exon) which encodes the extracellular domain of the CD28 protein. In an even more preferred embodiment, the spacer sequence targets exon 2 of the endogenous CD28-encoding gene (CD28-Ex2).

Preferably, the spacer sequence comprises or consists of the nucleotide sequence GCUUGUAGCGUACGACAAUG (SEQ ID NO: 50). In the herein disclosed examples, a crRNA having the 20nt spacer sequence 5′-GCUUGUAGCGUACGACAAUG-3′ (SEQ ID NO: 50) which targeted exon 2 of the endogenous CD28-encoding gene (CD28-Ex2) was particularly effective for achieving the CRISPR/Cas9-mediated disruption of the endogenous CD28-encoding gene. It is, however, expected by the inventors that spacer sequences which target other regions within the CD28-encoding gene, preferably also within exon 2, will also work, and corresponding embodiments are also contemplated.

In a preferred embodiment, the crRNA has the nucleotide sequence

(SEQ ID NO: 51)
GCUUGUAGCGUACGACAAUGGUUUUAGAGCUAUGCU.

The tracrRNA may be any tracrRNA capable of forming a functional RNP complex with the crRNA and a Cas9 nuclease. For example, the tracrRNA may be the natural tracrRNA from S. pyogenes or a functional fragment thereof. Preferably, the tracrRNA is a synthetic (non-natural) tracrRNA with increased stability and/or improved performance as compared to the natural tracrRNA from S. pyogenes, such as the “Alt-R® CRISPR-Cas9 tracrRNA” from Integrated DNA Technologies (IDT) (as used in Example 2). Other suitable tracrRNAs are available in the art and may alternatively be employed.

In accordance with a preferred embodiment of the method of the invention, a polynucleotide encoding (in expressible form) a Cas (e.g., Cas9) nuclease is introduced into the T cells in step (a) of the method of the invention, whereas in another preferred embodiment of the method of the invention, the Cas (e.g., Cas9) nuclease itself (i.e., in proteinaceous form) is introduced into the T cell.

In accordance with another preferred embodiment of the method of the invention, the Cas nuclease itself (i.e., in proteinaceous form) is introduced into the cells, however, in this case in form of a ribonucleoprotein (RNP) complex, i.e., a complex between Cas nuclease and a suitable crRNA and tracrRNA, or a complex between Cas nuclease and a suitable single guide RNA (sgRNA). RNPs can be assembled in vitro and subsequently delivered into the cell by methods known in the art, for example, electroporation or lipofection. RNPs are capable to cleave the target site with comparable efficacy as nucleic acid-based (e.g., vector-based) Cas nucleases (Kim et al. (2014), Genome Research 24(6):1012-1019).

Means for introducing (poly)peptides (and/or ribonucleoprotein (RNP) complexes) into living cells are known in the art and comprise, but are not limited to, microinjection, electroporation, lipofection (using liposomes), nanoparticle-based delivery, and protein transduction. Any one or more of these methods may be used in connection with the method of the invention. In this regard, the Cas (e.g., Cas9) nuclease to be introduced may either be isolated from their natural environment or recombinantly produced.

A liposome used for lipofection is a small vesicle, composed of a similar material as a cell membrane (i.e., normally a lipid bilayer, e.g., made of phospholipids), which can be filled with one or more (poly)peptide(s) (e.g., Torchilin V P (2006), Adv Drug Deliv Rev., 58(14):1532-55). To deliver a (poly)peptide into a cell, the lipid bilayer of the liposome can fuse with the lipid bilayer of the cell membrane, thereby delivering the contained (poly)peptide into the cell. It is preferred that the liposomes used in accordance with invention are composed of cationic lipids. The cationic liposome strategy has been applied successfully to (poly)peptide delivery (Zelphati O et al. (2001). J. Biol. Chem. 276, 35103-35110). As known in the art, the exact composition and/or mixture of cationic lipids used can be altered, depending upon the (poly)peptide(s) of interest and the cell type used (Felgner et al. (1994). J. Biol. Chem. 269, 2550-2561).

Protein/peptide transduction specifies the internalization of proteins/peptides into the cell from the external environment (Ford K G et al. (2001), Gene Therapy, 8:1-4). This method relies on the inherent property of a small number of proteins and peptides (preferably 10 to 16 amino acids long) to being able to penetrate the cell membrane. The transducing property of these molecules can be conferred upon proteins/peptides which are expressed as fusions with them and thus offer, for example, an alternative to gene therapy for the delivery of therapeutic proteins/peptides into target cells. Commonly used proteins or peptides being able to penetrate the cell membrane are, for example, the antennapedia peptide, the herpes simplex virus VP22 protein, HIV TAT protein transduction domain, peptides derived from neurotransmitters or hormones, or a 9×Arg-tag. Microinjection and electroporation are well known in the art and the skilled person knows how to perform these methods. Microinjection refers to the process of using a glass micropipette to introduce substances at a microscopic or borderline macroscopic level into a single living cell. Electroporation is a significant increase in the electrical conductivity and permeability of the cell plasma membrane caused by an externally applied electrical field. By increasing permeability, protein (or peptides or nucleic acid molecules) can be introduced into the living cell.

The Cas nuclease may be introduced into the cells as an active enzyme or as a proenzyme. In the latter case the Cas nuclease is biochemically changed within the cells (for example by a hydrolysis reaction revealing the active site or changing the configuration to reveal the active site), so that the proenzyme becomes an active enzyme. Possible means for introducing a nucleic acid molecule encoding (in expressible form) the Cas nuclease into cells are outlined herein below.

The polynucleotide encoding a CAR can be introduced into T cells by any known method suitable for integrative or non-integrative gene delivery. Exemplary gene delivery methodologies that are being applied in human gene therapy clinical trials for transferring chimeric antigen receptors (CARs) into T cells include viral vector-based gene transfer technologies, transposons, and mRNA electroporation as reviewed, for example, in: “Genetic Modification of T Cells”, Morgan R A, Boyerinas B., Biomedicines (2016); 4(2):9); “Engineering cell-based therapies to interface robustly with host physiology.” Schwarz K A, Leonard J N., Adv Drug Deliv Rev. (2016); 105(Pt A):55-65).

In a preferred embodiment, the polynucleotide encoding the CAR is introduced into the T cells by transfecting the T cells with a vector, preferably a viral vector.

The term “vector”, as used herein, refers to a polynucleotide molecule capable of transferring or transporting another polynucleotide molecule. The transferred polynucleotide is generally linked to, e.g., inserted into, the polynucleotide of the vector. A vector may include sequences that direct autonomous replication in a host cell or may include sequences sufficient to allow integration into the host cell genome. Exemplary vectors include, for example, plasmids (e.g., DNA plasmids or RNA plasmids), transposons, cosmids, bacterial artificial chromosomes, and viral vectors.

Useful viral vectors include, e.g., adenoviruses (AdVs), adeno-associated viruses (AVVs), retroviruses and lentiviruses (including replication defective retroviruses and lentiviruses). Preferred vectors, due to the ability of efficiently integrating into the genome of the transduced cells, are retroviral vectors, especially gamma(γ)-retroviral vectors and lentiviral vectors. The term “retrovirus”, as used herein, refers to an RNA virus that reverse transcribes its genomic RNA into a linear double-stranded DNA copy and subsequently covalently integrates its genomic DNA into a host cell genome. Exemplary retroviruses, include, but are not limited to: Moloney murine leukemia virus (Mo-MLV), Moloney murine sarcoma virus (Mo-MSV), Harvey murine sarcoma virus (Ha-MuSV), murine mammary tumor virus (Mu-MTV), gibbon ape leukemia virus (GaLV), feline leukemia virus (FLV), spumavirus, Friend murine leukemia virus (F-MuLV), Murine Stem Cell Virus (MSCV) and Rous Sarcoma Virus (RSV) and lentivirus.

The term “lentivirus”, as used herein, refers to a subgroup (genus) of the Retroviridae family. Lentiviruses are unique among the retroviruses in being able to infect non-dividing cells (and terminally differentiated cells); they can deliver a significant amount of genetic information into the genome of the host cell. Exemplary lentiviruses include, without being intended to be limiting, human immunodeficiency virus (HIV); simian immunodeficiency virus (SIV); Maedi-Visna virus (MVV); feline immunodeficiency virus (FIV); caprine arthritis-encephalitis virus (CAEV); equine infectious anemia virus (EIAV); and bovine immune deficiency virus (BIV).

It is generally understood that in cases where a viral vector has pathogenic activity, a corresponding vector will be rendered non-pathogenic by depletion of its pathogenic components before being employed in any of the herein disclosed embodiments.

As will also be understood by the person skilled in the art, the term “viral vector” is generally used to refer either to a nucleic acid molecule (e.g., a plasmid) that includes virus-derived nucleic acid elements that typically facilitate transfer of a polynucleotide of interest (i.e. a transgene, such as the CAR-encoding polynucleotide) or integration thereof into the genome of a host cell, or to refer to a viral particle that mediates the gene transfer. Viral particles will typically include various viral components and sometimes also host cell components in addition to nucleic acid(s).

The viral vector can be a virus (viral particle), which is used to infect cells. Following infection, at least a portion of the viral genome or a copy thereof integrates into the cellular genome, typically at random sites within the genome of the cell.

In a preferred embodiment of the method of the invention, step (b) comprises:
(b-i) transducing the T cells obtained in step (a) with a viral vector comprising the CAR-encoding polynucleotide; and
(b-ii) culturing and expanding the transduced T cells in vitro; and/or optionally
(b-iii) enriching T cells for the presence or absence of a surface-molecule, preferably enriching CD28⁻ T cells.

In preferred embodiments, a retroviral vector is employed for introducing a polynucleotide encoding a chimeric antigen receptor (CAR) into the T cells. In other words, the viral vector used in step (b-i) is preferably a retroviral vector. Retroviral vectors and methods of generating and using retroviral vectors to introduce an exogenous gene (e.g., a CAR-encoding polynucleotide) into mammalian cells, such as T cells, are well known in the art (see, e.g., Vargas J E et al. J Transl Med. 2016; 14: 288) and are also described herein. In a typical setup, T cells (e.g., from a donor) are transduced with a retroviral vector comprising the CAR-encoding polynucleotide. The retroviral vector uses the viral machinery to attach to the T cells, and, upon cellular entry, the retroviral vector introduces genetic material (including a CAR-encoding polynucleotide, flanked by the viral long terminal repeats (LTRs)) in the form of RNA. The RNA is then reverse-transcribed into double-stranded DNA by the virus-encoded reverse transcriptase. The double-stranded DNA, comprising the CAR-encoding polynucleotide, flanked by the LTRs, is then integrated into the T cell genome through the action of a second virus-encoded enzyme, integrase (IN). The 5′ LTR sequence typically includes a strong promoter region containing several cis elements for transcription-factor binding and a highly active initiator sequence. The 3′ LTR typically acts as the termination and polyadenylation site. Thus, as the polynucleotide encoding the CAR is chromosomally integrated, the CAR is expressed by the T cell, and its expression will be maintained as the T cells divide.

A retroviral vector typically comprises long terminal repeats (LTRs) (i.e., the 5′ and 3′ LTRs), which can be derived from various types of retroviruses as known in the art and exemplary representatives are referred to herein. LTR(s) may be genetically modified to provide desired properties, and the viral genome can be modified, e.g., to lack promoter activities and/or to comprise regulatory elements suitable for propagation and selection in bacteria, such as an origin of replication and an antibiotic resistance marker. The transgene cassette (comprising a polynucleotide encoding a CAR) is positioned between the LTRs. Infectious, replication-competent retroviral particles can be produced by transfecting a retroviral plasmid comprising the transgene cassette into a retrovirus packaging cell line using standard methods. The packaging cells are cultured, and viral particles released into the media are collected (e.g., as supernatants) for subsequent use, e.g., to infect mammalian target cells (e.g., T cells). Upon infection, the transgene cassette integrates (randomly) into the genome of the target cell.

Exemplary retroviral vectors may be derived from any one or more known retroviruses, such as, without being intended to be limiting, Moloney murine leukemia virus (Mo-MLV), Moloney murine sarcoma virus (Mo-MSV), murine myeloproliferative sarcoma virus (MPSV), and lentiviruses that are capable of integrating into the genome of a host cell.

One particularly preferred retroviral vector which may be employed in the method of the invention is the retroviral vector plasmid pMP71 (see Example 2). The MP71 retroviral vector combines MPSV-LTR promoter-enhancer sequences and improved untranslated sequences derived from the murine embryonic stem cell virus (MESV) and has previously been shown to mediate high transgene expression in T cells (cf. Hildinger M et al. (1999). J. Virol. 73, 4083-4089; Engels B et al., (2003) Human Gene Therapy 14:1155-1168).

In some embodiments, the retroviral vector may be replication-defective (e.g., essential genes for viral replication, e.g., genes encoding virion structural, replicatory and DNA modifying proteins, are deleted or disabled). The virion structural, replicatory, and DNA modifying proteins are provided in trans during viral packaging in a packaging cell line, through transient co-transfection of nucleic acids encoding the virion proteins (e.g., the integrase protein is encoded on another DNA vector), or as a recombinant protein.

In general, the retroviral vector includes one or more genes of interest flanked by “long terminal repeat” or “LTR” sequences (i.e., the 5′ and 3′ LTRs). Exemplary LTRs include, but are not limited to, Mo-MLV, Mo-MuSV, MMTV, HIV, and equine infectious anemia LTRs. The LTRs may contain sequences required for the association of viral genomic RNA, reverse transcriptase and integrase functions, and sequences involved in directing the expression of the genomic RNA to be packaged in viral particles.

It is well known that the host range of retroviral vectors, including lentiviral vectors, can be expanded or altered by a process known as pseudotyping. Pseudotyped retroviral vectors consist of vector particles bearing envelope glycoproteins (GPs) derived from other enveloped viruses. The resulting viral particles then possess the tropism of the virus from which the GP was derived. Thus, by pseudotyping the retroviral vector with selected virus-derived envelope GPs, the natural tropism of a certain virus can be exploited for targeting a certain type of host cells or host tissue.

Various viral envelope glycoproteins (GPs) suitable for pseudotyping are commonly employed in the art, including without limitation, envelope GPs from gibbon ape leukemia virus (GALV), feline RD114 virus (RD114), vesicular stomatitis virus (VSV), Piry virus, Chandipura virus, Spring viremia of carp virus (SVCV), and Mokola virus (MV) or engineered variants thereof.

In the present case, the retroviral vector can be pseudotyped with any viral envelope GP suitable for targeting T cells.

In a preferred embodiment, the retroviral vector is pseudotyped with the feline RD114 envelope glycoprotein (RD114 GP). In the herein disclosed examples, pseudotyping with RD114 GP was particularly effective for retroviral transduction of T cells. However, other viral envelope GPs may also be suitable and may alternatively be employed for the herein disclosed purposes.

In a more preferred embodiment, the retroviral vector is a MPSV-derived vector pseudotyped with RD114 GP.

The term “transducing” or “transduction”, as used herein in the context of (retro-)viral-based transgene delivery is well known in the art and refers to the process of introducing genetic material (a CAR-encoding polynucleotide) into a cell and, optionally, its subsequent integration into the genome of said cell, via viral vector particles. Generally, the step of transducing a retroviral vector into T cells comprises contacting (i.e., inoculating) the retroviral vector with the T cells. Means and methods for transducing a retroviral vector into T cells are well known in the art. It is also known that the transduction efficiency can be enhanced, for example, by applying a so-called “spinoculation” step during the transduction, i.e., to subjecting the T cells to a centrifugation while being inoculated with the viral vector. It is also known that the transduction efficacy can be enhanced by using retronectin (a recombinant human fibronectin that mediates interaction between mammalian host cells and retroviral vector, thus enhancing the transduction efficiency).

Thus, in a particularly preferred embodiment of the method of the invention, the step of transducing the T cells with the retroviral vector is conducted (i) by spinoculation, (ii) in the presence of retronectin and/or (iii) at a temperature between 27° C. and 37° C., preferably at 32° C. In a preferred embodiment, the spinoculation is conducted by applying a centrifugation (preferably, at 450 g for 10 min and/or at 32° C.) during transduction.

Other viral vectors and transduction methods also have been described in the art, including lentivirus systems (e.g., human immunodeficiency virus 1 and 2 (HIV-1 and HIV-2), simian immunodeficiency virus (SIV), feline immunodeficiency virus (FIV), equine infectious anemia virus (EIAV), caprine arthritis encephalitis virus (CAEV), Visna virus, and Jembrana disease virus (JDV)). Lentiviral vectors and systems are of interest because of their ability to infect non-dividing, terminally differentiated cells and to insert into the genome though an integrase-based mechanism.

In alternative embodiments, a polynucleotide encoding a CAR is introduced into T cells (i.e., chromosomally integrated) by homology directed repair (HDR), preferably, by CRISPR/Cas9-mediated HDR. In a particular preferred embodiment of the latter embodiment, the CAR-encoding polynucleotide is integrated into the endogenous CD28 encoding gene, thereby causing the disruption of the endogenous CD28-encoding gene. Methods for conducting HDR at desired chromosomal positions are well known in the art.

In a further preferred embodiment of the method of the invention, step (b) is conducted within a time period of, with increasing preference, between 0 hours and 144 hours, between 6 hours and 120 hours, between 24 hours and 114 hours, between 48 hours and 108 hours, between 72 hours and 104 hours, between 78 hours and 102 hours, between 84 hours and 100 hours, and most preferably between 92 hours and 100 hours,
(i) after the T cells were obtained from the donor's body; and/or
(ii) after the initiation of the activation in step (0-aiii).

In another preferred embodiment, step (b) is conducted (i.e., initiated) at a time point of about 96 hours (+/−6 hours) after the initiation of the activation in step (0-aiii).

In a further preferred embodiment of the method of the invention, the method further comprises, prior to (b), one or more of the following steps:
(0b-i) enriching the T cells for one or more T-cell sub-populations based on surface marker expression and/or antigen-binding specificity; and/or
(0b-ii) activating the T cells by contacting the T cells in vitro with one or more T-cell-activating agents; and optionally removing the one or more T-cell-activating agents after activation.

Since in the T cells, in step (a) of the method of the invention, the endogenous CD28-encoding gene is disrupted, it is understood that in optional step (0b-ii) T-cell activation is conducted by application of other T-cell-activating agents than CD28 agonists. Alternative T cell activating agents are known in the art and are also described herein above.

In alternative embodiments, the polynucleotide encoding the CAR is introduced into the T cells by means of transposon-based gene delivery (also commonly referred to a “transposon-based insertional mutagenesis (TIM)”); wherein preferably, the polynucleotide encoding the CAR is introduced into the T cells by transfecting the T cells with a DNA transposon-based gene delivery vector, wherein the DNA transposon-based gene delivery vector comprises the CAR-encoding polynucleotide. In particular preferred embodiments, the DNA transposon-based gene delivery is based on a Sleeping Beauty (SB) transposon system. Means and methods for (DNA) transposon-based transgene delivery, including Sleeping Beauty (SB) transposon vector systems and their application in T cell engineering and CAR T cell immunotherapy are known in the art; and described, for example, in review articles “Transposon-mediated genome manipulation in vertebrates.” by Ivics Z et al., Nat Methods. (2009); 6(6):415-22); Aronovich E L et al., Hum Mol Genet. (2011); 20(R1):R14-20; Magnani C F et al., Cells. (2020); 9(6):1337.

In preferred embodiments, the method of the invention further comprises a step of detecting and/or enriching CAR-expressing modified T cells (e.g., after step (b) and/or as a final method step), for the sake of a quality control or for determining a suitable dose for administration to a patient. This may, in certain instances, be facilitated by applying one or more cell capturing and/or cell sorting approaches, wherein an interaction either with the CAR, preferably the epitope-tag present in the ectodomain of the CAR, or an epitope-tag expressed as separate (poly)peptide on the surface of the T cells will be exploited. Exemplary approaches are discussed above in connection with the embodiments pertaining to epitope-tags and in the examples (see, e.g., Example 5).

In certain embodiments, a detection of CAR-expressing T cells (e.g., for assessing the efficiency of the transduction) is established by conducting the following in vitro method, the method comprising:
(i) contacting the modified T cells (e.g., obtained in step (b)) with a recombinant CD28 (poly)peptide, wherein the recombinant CD28 (poly)peptide further comprises a label, and allowing binding of the recombinant CD28 (poly)peptide to the CD28-binding antigen binding moiety;
(ii) washing the T cells for removal of unbound recombinant CD28 (poly)peptide; and
(iii) detection of the presence or absence of the label, wherein the detected presence of the label indicates an expression of the CAR on the modified T cells.

Preferably, the recombinant CD28 (poly)peptide comprises the ectodomain of CD28 or a fragment thereof, wherein the fragment is of sufficient length for being bound by the CD28-specific antigen binding moiety of the CAR. Preferably, the recombinant CD28 (poly)peptide is soluble at aqueous (preferably physiological) buffer conditions. Preferably, the label is itself detectable (i.e., a detectable label), preferably a conjugated fluorophore. Alternatively, the label is a (poly)peptide tag (e.g., a poly-His-tag). Detection of the bound recombinant CD28 (poly)peptide would then require additional use of a tag-specific binding protein (preferably an antibody, e.g., an anti-His-tag antibody) which is either itself detectable or conjugated with a detectable label, such as a fluorophore.

One particularly preferred implementation of such a detection method (staining method) developed by the inventors is presented in Example 5.

In cases where it is intended to obtain modified T cells capable of co-targeting additional targets (e.g., CD7) besides targeting CD28, the method may be further adapted to also effecting a disruption of the respective additional endogenous gene encoding that target (e.g., the endogenous CD7-encoding gene) in said T cells; and to effecting introduction into said T cells a polynucleotide encoding an antigen binding moiety that is capable of specific binding to that additional target (e.g., the extracellular portion of CD7).

Embodiments are contemplated herein, wherein the polynucleotide encoding that further antigen binding moiety is comprised in the same polynucleotide encoding the antigen binding moiety which binds to CD28 in order to be expressed (i) as a further component of the ectodomain of the CAR molecule; or (ii) as part of the ectodomain of a different CAR molecule. In alternative embodiments, the polynucleotide encoding the further antigen binding moiety is comprised in a further polynucleotide which is not comprised in the first polynucleotide that encodes the CAR comprising the CD28-binding antigen binding moiety; wherein preferably, the polynucleotide encoding the further antigen binding moiety is comprised in a polynucleotide encoding a further CAR to be expressed as part of the ectodomain of that further CAR molecule. The skilled person, in view of the present disclosure, will be able to implement the necessary adaptions of the method by employing analogous means and activities as described herein in connection with the method for generating modified T cells having a disrupted endogenous CD28-encoding gene and a polynucleotide encoding a CD28-targeted CAR.

In a fourth aspect, the invention relates to modified T cells obtained by the method according to the third aspect of the invention.

In a fifth aspect, the invention relates to modified T cells according to the invention for use as a medicament.

In a sixth aspect, the invention relates to modified T cells according to the invention for use in treating a T cell-mediated disorder or other disorder which will benefit from an elimination of CD28-expressing-cells; preferably selected from
(a) a T-cell hyperproliferative disorder; and/or
(b) T-cell lymphoma (TCL), T-cell non-Hodgkin lymphoma (T-NHL), mycosis fungoides, anaplastic large cell lymphoma (ALCL), cutaneous T-cell lymphoma (CTCL), peripheral T-cell lymphoma (PTCL), precursor T-lymphoblastic lymphoma (Pre-T-LBL), T-cell acute lymphoblastic lymphoma (T-LBL), and/or angioimmunoblastic T cell lymphoma (AITL); and/or
(c) T-cell leukemia (TLL), acute myeloid leukemia (AML), T-cell acute lymphoblastic leukemia (T-ALL), pediatric T-ALL, adult T-ALL, T-cell prolymphocytic leukemia (T-PLL), T-cell large granular lymphocyte (T-LGL) leukemia, and/or adult T cell lymphoma-leukemia (ATL); and/or
(d) a T-cell-mediated autoimmune disease; and/or
(e) Non-Hodgkin Lymphoma (NHL); and/or
(f) a B-cell hyperproliferative disorder, preferably multiple myeloma (MM); and/or
(g) any other disorder characterized by CD28-expressing disease-promoting cells.

In a preferred embodiment, the disorder is mediated by T cells and/or B cells that are CD28⁺; and wherein optionally
(a) said T cells are CD2⁻, CD5⁻, CD7⁻, CD30⁻, CD37⁻, and/or CCR4⁻; and/or
(b) said B cells are CD19⁻; and/or
(c) said T cells and/or said B cells are resistant to anti-X CAR T cell immunotherapy, wherein X is a cell surface antigen distinct from CD28; and/or
(d) said T cells and/or said B cells are resistant to treatment with one or more chemotherapeutics.

In another preferred embodiment, the modified T cells are
(a) to be co-administered with
   (i) an anti-X antibody or antibody-drug-conjugate, wherein X is a surface antigen distinct from CD28; wherein X is preferably CD2, CD5, CD7, CD28, CD30, CD37 or CCR4; and/or
   (ii) an anti-X CAR T cell immunotherapy, wherein X is a surface antigen distinct from CD28; wherein X is preferably CD2, CD5, CD7, CD28, CD30, CD37 or CCR4; and/or
   (iii) one or more inhibitors of T-cell inhibitory signaling, preferably an anti-PD-1 antibody and/or anti-CTLA4 antibody; and/or
(b) to be administered prior to or after
   (i) a chemotherapeutic treatment, wherein the chemotherapeutic is preferably one or more of cyclophosphamide, doxorubicin (Adriamycin), vincristine, L-asparaginase, methotrexate, prednisone, and/or cytarabine (ara-C); and/or
   (ii) stem cell transplantation, preferably after chemotherapeutic treatment and prior to stem cell transplantation.

In particularly preferred embodiments of the latter embodiment, the modified T cells are to be co-administered with (i) an anti-CD7 antibody and/or anti-CD7 antibody-drug-conjugate; and/or (ii) an anti-CD7 CAR T cell immunotherapy. The feasibility of co-targeting CD28 and CD7 is demonstrated in Example 6 and considered by the inventors as a promising route for the treatment of any T cell or B cell malignancies characterized by the surface expression of these two antigens, such as T cell precursor childhood leukemia, including, inter alia, pediatric T-ALL and related forms thereof.

In another preferred embodiment, the subject will benefit from a selective depletion of CD28-expressing cells, preferably CD28-expressing T-cells and/or CD28-expressing B-cells (including CD28-expressing plasma cells). It is shown in the herein disclosed data (cf. FIG. 1) that CD28 is overexpressed in certain T-lineage malignancies, such as T-cell acute lymphoblastic leukemia (T-ALL), particularly in pediatric T-ALL (see FIG. 1B). Besides, CD28 is also known to being highly expressed on B cells in certain B-cell malignancies, such as multiple myeloma (MM). In view of their observed capacity to selectively deplete CD28⁺ T cells, while sparing CD28⁻ T cells (FIG. 5D) and maintaining proliferation capacity (FIG. 5C), the modified T cells of the invention are expected to provide a potent therapeutic means specifically in malignancies characterized by CD28-overexpressing T cells, such as (pediatric) T-ALL and NHL (Non-Hodgkin Lymphoma), adult T-ALL and NHL, multiple myeloma (MM) and any other CD28-expressing disease-promoting cells.

Moreover, it is known from previous studies that a significant proportion of patients (˜10%) with various T lineage malignancies, such as cutaneous T cell lymphoma (CTCL), angioimmunoblastic T cell lymphoma (AITL), peripheral T cell lymphoma (PTCL) and adult T cell lymphoma-leukemia (ATL), have acquired mutations in the extracellular portion of CD28, and evidence has been found that some of these mutations have the potential to contribute to the disease pathology. For example, certain mutant isoforms of CD28 were found to increase binding to both CD80 and CD86 (i.e., the natural ligands of CD28) and that this can result in increased T cell proliferation and IL-2 secretion (Gmyrek, G B et al. (2017) Cell Immunol 319: 28-34). Thus, embodiments are contemplated herein wherein the modified T cells comprise a CAR which comprises in its ectodomain at least one antigen binding moiety that is capable of specific binding to the extracellular portion of one or more of the mutant isoforms of CD28 (e.g., CD28-F511, CD28-F51V) specifically observed in one or more of the aforementioned or other disorders. Preferably, a corresponding antigen binding moiety selectively binds to the extracellular domain of the respective mutant form(s), i.e., it does not specifically bind to the wild-type CD28. A corresponding setup will allow to selectively target and eradiate only such T cells expressing the mutated CD28 on their surface. Means and methods of developing suitable antigen binding moieties (e.g., a scFv) which selectively bind to a CD28 mutant are known in the art.

In a sixth aspect, the invention relates to the use of the modified T cell(s) of the invention, or of the population of modified T cells of the invention, for selective depletion of CD28⁺ cells in a sample in vitro.

In a typical envisaged therapeutic setting, modified T cells are generated according to the method of the invention for each prospective recipient (i.e., a patient) in an individualized (i.e., personalized) manner, using individually selected T cells, e.g., from a suitable source (e.g., a (partially) HLA-matched healthy donor). Yet, as a routine measure, the modified T cells will, prior to their actual administration, be subjected to an in vitro assay, wherein the modified T cells are contacted with prospective target cells (i.e. T cells and/or B-cells) from the prospective recipient (or with a standardized T cell population) in order to assessing their activity/cytotoxic capacity and hence their efficacy, safety, and/or longevity in the patient; and/or to determine a suitable dose. A corresponding application is defined by the use according to the sixth aspect of the invention.

The present invention relates in a seventh aspect to a method of treating a T-cell-mediated disorder, a B-cell-mediated disorder, and/or a disorder characterized by CD28-expressing disease promoting cells. Thus, it is understood that the method of treating according to the seventh aspect of the invention may be directed to treating any of the disorders mentioned herein, e.g., in connection with any of the referred (further) medical uses as envisaged herein including in particular those specified in embodiments disclosed in connection with the fifth and sixth aspect of the invention.

The modified T cells of the invention may be administered either alone, or as a pharmaceutical composition in combination with diluents and/or other components, such as, without limitation, one or more interleukins (e.g., IL-2) or other cytokines or cell populations. Briefly, pharmaceutical compositions of the present invention may comprise modified T cells as described herein, in combination with one or more pharmaceutically or physiologically acceptable carriers, diluents or excipients. Such compositions may comprise buffers such as neutral buffered saline, phosphate buffered saline (PBS) and the like; carbohydrates such as glucose, mannose, sucrose or dextran(s), mannitol; (poly)peptides or amino acids, such as glycine; antioxidants; chelating agents such as EDTA or glutathione; adjuvants (e.g., aluminum hydroxide); and/or preservatives. Compositions of the present invention are preferably formulated for intravenous administration.

The modified T cells or corresponding pharmaceutical compositions of the invention may be administered in any manner appropriate to the disease or disorder to be treated (or prevented). The quantity and frequency of administration will be determined by such factors as the condition of the patient, and the type and severity of the patient's disease, although appropriate dosages may be determined by clinical trials.

The dosage of the modified T cells to be administered to a patient will vary with the precise nature of the condition being treated and the recipient of the treatment. The scaling of dosages for human administration can be performed according to art-accepted practices.

The modified T cells may be administered in effective doses. The effective dose may be either one or multiple doses, and sufficient to produce the desired therapeutic effect. A typical dose of modified T cells may range from about 1×10⁵ to 5×10⁸ cells/kg body weight (bw) of subject receiving (similar as the doses discussed for various CD19-targeting CAR-T cell therapies in Hay K A & Turtle C J, Drugs. (2017); 77(3): 237-245). However, deviations from these indicated numbers may also be possible. The effective dose may be readily determined by the skilled person and/or may be calculated based on the stage of the malignancy, the health of the subject, and the type of malignancy. In the situation where multiple doses are administered, that dose and the interval between the doses may be determined based on the subject's response to therapy. The modified T cells or corresponding pharmaceutical compositions may be administered multiple times at these dosages.

The modified T cells or corresponding pharmaceutical compositions can be administered by using infusion techniques that are commonly known in immunotherapy (see, e.g., Rosenberg S A et al., (1988) New Eng. J. of Med. 319; 1676). The optimal dosage and treatment regime for a particular patient can readily be determined by one skilled in the art of medicine by monitoring the patient for signs of disease and adjusting the treatment accordingly.

The administration of the modified T cells or corresponding pharmaceutical compositions may be carried out in any convenient manner, including by injection, transfusion, implantation or transplantation. The modified T cells or corresponding pharmaceutical compositions described herein may be administered to a patient subcutaneously, intradermally, intratumorally, intranodally, intramedullary, intramuscularly, by intravenous (I.V.) injection, or intraperitoneally. In one embodiment, the modified T cells or corresponding pharmaceutical compositions are to be administered to a patient by intradermal or subcutaneous injection. In another embodiment, the modified T cells or corresponding pharmaceutical compositions are to be administered by I.V. injection.

In certain embodiments, the modified T cells or corresponding pharmaceutical compositions may, prior to their administration, be activated in vitro and/or expanded in vitro to therapeutic levels using methods described herein, or other methods known in the art.

For example, in a particular preferred embodiment of the therapeutic applications/medical uses described herein, the original T cells from which the modified T cells according to the invention are generated are autologous T cells (i.e., from the patient obtained, for example, by (lymph)apheresis)), which, after being subjected to the genetic engineering (CD28-KO and delivery of the CAR-encoding gene) as described in embodiments of the modified T cell or the method of the invention, are expanded in vitro to therapeutic numbers prior to being administered to the patient.

In other embodiments of the therapeutic applications/medical uses described herein, the modified T cells are to be administered at a dose not providing an immediate therapeutic effect; and a therapeutic effect will be provided following expansion of the modified T cells in vivo (i.e. in the patient's body).

The modified T cells or corresponding pharmaceutical compositions of the invention may be administered to a patient in conjunction with (e.g., before, simultaneously or following) any number of relevant treatment modalities, including but not limited to, treatment with agents (including small molecules, peptides or proteins, or engineered cells) such as antiviral therapy, chemotherapy, radiation, antibodies, anti-body drug conjugates, or CAR-T cells (other than the modified T cells of the inventions).

In a further embodiment, the modified T cells (or corresponding pharmaceutical composition) are to be administered to a patient in conjunction with (e.g., before, simultaneously or following) bone marrow transplantation, T cell ablative therapy using either chemotherapeutic agents such as, fludarabine, external-beam radiation therapy (XRT), cyclophosphamide, or antibodies.

In another embodiment, the modified T cells (or corresponding pharmaceutical composition) are to be administered following B cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded modified T cells or a corresponding pharmaceutical composition. In an additional embodiment, expanded modified T cells or a corresponding pharmaceutical composition are/is administered before or following surgery.

It is understood that the definitions and embodiments as described above in the context of the first aspect of the invention also apply, in as far as possible, mutatis mutandis to the second, third, fourth, fifth, sixth and seventh aspects of the present invention.

The invention is herein described, by way of example only, with reference to the accompanying drawings for purposes of illustrative discussion of the preferred embodiments of the present invention.

Unless otherwise defined, all technical and scientific terms used herein have the same meaning as commonly understood by one of ordinary skill in the art to which this invention belongs. In case of conflict, the patent specification, including definitions, will prevail.

Regarding the embodiments characterized in this specification, in particular in the claims, it is intended that each embodiment mentioned in a dependent claim is combined with each embodiment of each claim (independent or dependent) said dependent claim depends on. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

Modifications may be made to the foregoing without departing from the basic aspects of the technology. Although the technology has been described in substantial detail with reference to one or more specific embodiments, those of ordinary’ skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The terms “method” and “process” are used interchangeably herein.

The term “a” or “an” can refer to one of or a plurality of the elements it modifies (e.g., “a cell” can mean “one or more cells”) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

The Figures show:

FIG. 1: Expression of CD28 on T-lineage acute lymphoblastic leukemia. (A) CD28 mRNA expression data from the Leukemia MILE Study (Haferlach et al., 2010) generated from the webtool bloodspot.eu.²³ Overexpression of CD28 in patients (n=174) compared to controls is documented. (B) CD28 expression on primary blasts from pediatric T-ALL patients at time of initial diagnosis compared to healthy bone marrow progenitors. (C) Detection of CD28 on T-cell precursors within the bone marrow of a healthy donor and on a T-ALL patient.

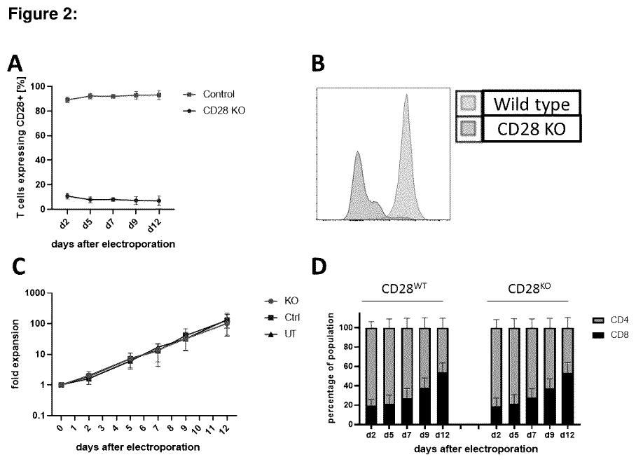

FIG. 2: CD28 knockout in primary T cells does not affect T cell expansion or CD4/CD8 ratio. (A) After CRISPR/Cas9 mediated Knockout of CD28 or mock electroporation of primary T cells, CD28 expression was measured by flow cytometry over 12 days: while ˜90% of activated T cells express CD28 (Control), only ˜10% of CD28 knockout (CD28 KO) cells express CD28. (B) Histogram overlay of CD28 flow cytometry in either CD28^{WT} or CD28^{KO} T cells. (C) Expansion of T cells after either CD28 knockout (KO), mock electroporation (Ctrl) or non-electroporated T cells (UT). (D) CD4/CD8 compartments were quantified using flow cytometry after mock electroporation (CD28^{WT}) or CD28 KO (CD28^{KO}). This graph illustrates data from biological triplicates.

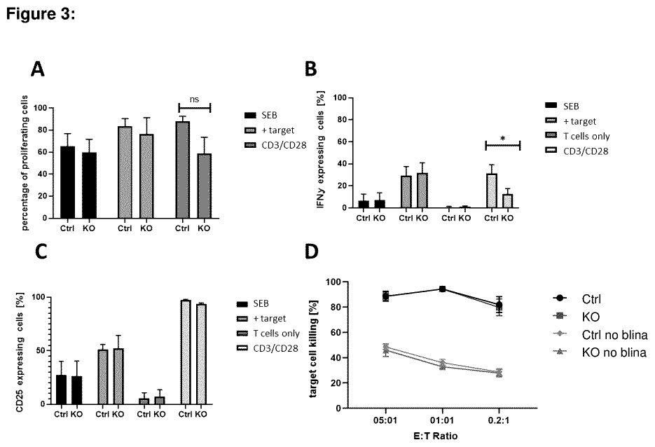

FIG. 3: CD28 knockout in primary T cells does not lead to functional impairment. Either CD28^{WT} (Ctrl) or CD28^{KO} (KO) T cells were stimulated with different agents (Staphylococcal enterotoxin B: SEB, with CD19⁺ target cell line and the CD3/CD19 bispecific T cell engager blinatumomab: + target and stimulation of CD3/CD28 with monoclonal antibodies) and subsequently analyzed. The percentage of proliferating cells was unchanged after stimulation with SEB or CD19⁺ target cells, while there was a trend towards less proliferation after CD3/CD28 stimulation (A). In line with this finding, expression of IFNy as measured by intracellular cytokine staining, was reduced only after activation by CD3/CD28 antibodies (B). Both expression of activation marker CD25 and killing of CD19⁺ target cells in blinatumomab co-culture assays were unchanged by CD28 knockout (C&D).

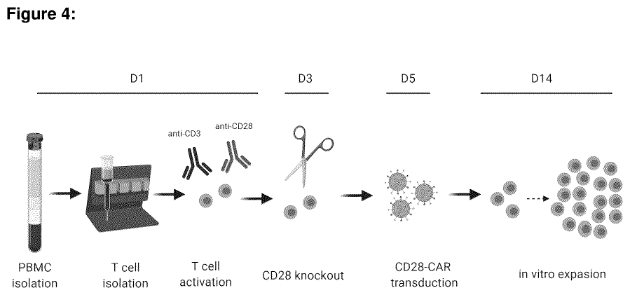

FIG. 4: Experimental setup for testing of CD28-CAR T cells. Peripheral blood mononuclear cells were isolated from healthy donors and after isolation of CD3⁺ T cells, CD3/CD28 activation was performed. After 48 hours, activated T cells were either mock electroporated or CRISPR/Cas9 knockout of CD28 was performed. Again 48 hours later, retroviral transduction with CD28_CAR molecules was performed and T cells were subsequently expanded until 14 days after activation.

FIG. 5: Expression and functional characterization of the five best CD28-CAR molecules on primary T cells. (A) Surface expression of CD28_CAR_2 molecule 12 days of retroviral transduction; similar transduction rates were reached with other constructs. (B) Expression of CD28 on T cells either 12 days after mock transduction (left panel, UT) or 12 days after transduction of CD28_CAR_2 (right panel). (C) Expansion of T cells transduced with CD28_CAR_2 and CD28 knockout (ko) or mock electroporation (ctrl), of T cells not electroporated and CD28_CAR_2 transduced (td) or mock-transduced and not electroporated after activation (ut). (D) Both functional (CD28_CAR_1, CD28_CAR_2, CD28_CAR_11, CD28_CAR_12 and CD28_CAR_14) and non-functional (CD28_CAR_5, CD28_CAR_6, CD28_CAR_8, CD28_CAR_16) CD28_CARs and mock transduced T cells (UT) were tested for cytotoxicity against CD28⁺ CCRF-CEM^{WT} and CD28⁻ CCRF-CEM^{CD28KO} target cells. Clearly, specific killing observed by CD28_CAR_1, CD28_CAR_2, CD28_CAR_11, CD28_CAR_12 and CD28_CAR_14 when tested against CCRF-CEM^{WT} is abolished by CD28 knockout in CCRF-CEM cells (right panel). (E) Expansion of T cells after CD28 knockout and transduction of one out of five CD28_CAR constructs and of a CD19-CAR either with CD28 knockout (CD19_CAR_28KO) or without CD28 knockout (CD19_28_control) and mock transduced T cells (UT).

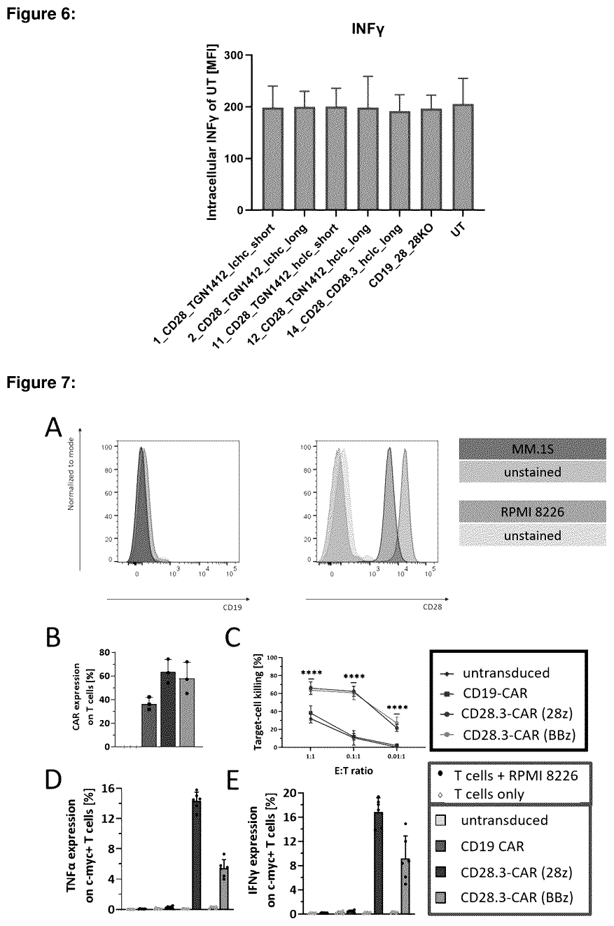

FIG. 6: Binding of CD28 on T cells by anti-CD28 CAR-T cells does not lead to activation of target T cells. No increased interferon gamma (IFNy) release by wild-type (wt) T cells upon co-culture with TGN1412-scFv-containing CD28-targeting CAR T cells. After in vitro expansion for 14 days including CD28 CRISPR/Cas9 Knockout at day 3 after activation, both untransduced, wild-type T cells and CAR T cells containing different genetic constructs (CAR-1, CAR-2, CAR-11, CAR-12 and CAR-14, CD19-CAR) were cryopreserved in 10% DMSO. After thawing, resting o/n at 37° C. and 5% CO₂ and CD56 depletion the following experiment was performed. Next, untransduced T cells were stained with a labelling dye (cell trace violet, ® ThermoFisher) and subsequently 20.000 labelled, untransduced T cells were co-cultured with 20.000 non-labelled CD28 CAR T (mean transduction rate: 70%) cells for 24 hours. After co-culture period, intracellular interferon gamma (IFNy) levels were measured 2 hours after Golgi-Stop treatment with Brefeldin A. Results indicate median fluorescence intensity of IFNy from three independent donors.

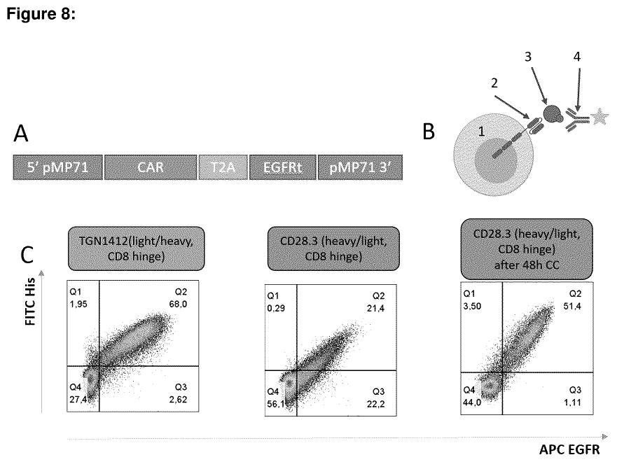

FIG. 7: Functionality of CD28 CAR cells against multiple myeloma. CD28 CAR T cells detect CD28 expression on multiple myeloma cell lines and are superior to CD19 CAR T cells in killing multiple myeloma cell lines. (A) Flow cytometric detection of CD19 or CD28 expression on two multiple myeloma cell lines, (RPMI 8226 and MM.1S cells) reveals that CD19 expression is scarce on both analyzed multiple myeloma cell lines, while both cell lines express CD28. (B) Transduction rates of CD19 and CD28 CAR T cells was above 30% in all samples used. (C) CD28 CAR T cells show superior target killing as compared to both, untransduced T cells and CD19 CAR T cells. (D&E) Intracellular cytokine staining of CAR T cells for TNFα and IFNy demonstrate the specific detection of RPMI 8226 by CD28 CAR T cells, while no signal is present in the absence of target cells or in CD19 CAR T cells. These results demonstrate that CD28 CAR T cells are capable of detecting CD28 expression on and effective (and superiorly effective compared to CD19 CAR T cells) in killing multiple myeloma cell lines.

FIG. 8: Alternative approaches for detection of CD28 CAR transduced T cells. Exemplification of two further alternative (Myc-tag independent) staining methods for detection of CD28 CAR molecule expression on transduced T cells: (A) A truncated epidermal growth factor receptor (EGFRt)-linker molecule was inserted into the expression cassette as described previously.³¹ (B) A two-step staining protocol was developed: a T cell (1) expressing a CD28 CAR molecule (2) was first incubated for 30 min at room temperature with recombinant CD28 protein (3) with a poly-His tag (red dot). After a washing step, an anti-His-antibody (4) labelled with a fluorophore was added, thus facilitating tag-free CAR T cell detection. (C) For some constructs, tag-free staining and EGFRt staining led to the same transduction rates (see TGN1412 construct in the left panel). However, in other constructs (see CD28.3 construct presented here (middle panel: before target-co-culture/right panel: after target co-culture), only after target co-culture, the same transduction rates were observed by EGFRt expression and direct CAR staining. In summary, three different CAR staining protocols were employed for CD28 CAR T cells: some enable the use of monoclonal antibodies as a “safety switch” in a clinical setting (EGFRt), while others make the CAR design simpler which is beneficial in technical and regulatory concerns.

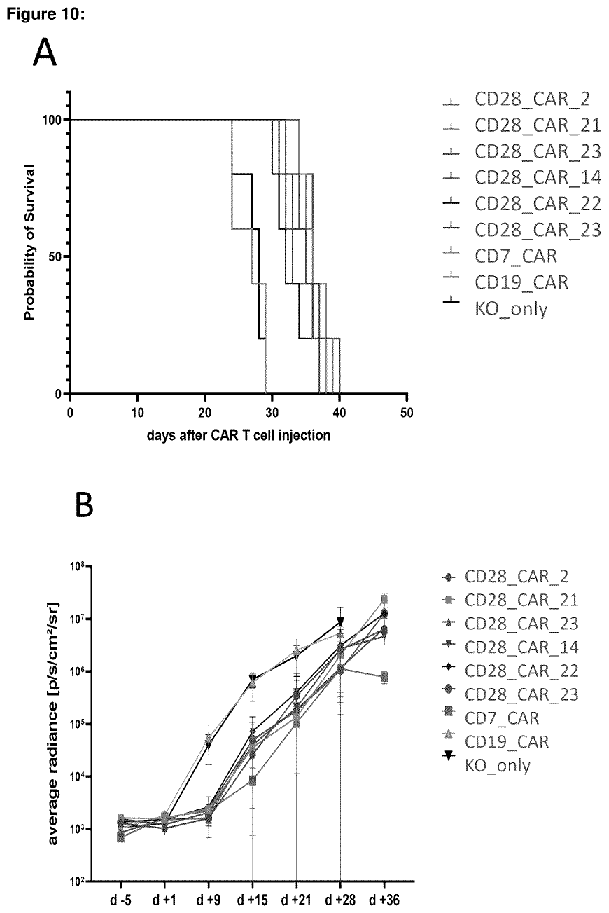

FIG. 9: Combination of CD28 CAR T cells with CAR T cells specific for alternative antigens. Comparison of the cytotoxic capacities of CD28 CAR T cells vis-à-vis CD7 CAR T cells, and feasibility of co-targeting of CD7 and CD28 by CAR T cells. (A) Transduction rates of two CD28 CARs and of one CD7 CAR construct on physiologic T cells after 14 days of expansion in vitro (n=4). (B) Cytotoxicity of these three CAR T cell populations against Jurkat TCP-ALL cell line: at a E:T ratio of 0.04:1, CD28.3 CD28 CAR T cells show a higher cytotoxicity than both TGN1412 CD28 CAR T cells and CD7 CAR T cells, while no difference can be seen between all constructs tested at higher E:T ratios. (C) Knockdown of CD7 and CD28 is feasible in primary T cells using CRISPR/Cas9. These results show (i) that CD28 CARs and CD7 CARs provide comparable cytotoxicity against TCP-ALL cell lines; (ii) that CD7/CD28 double-knockout T cells can be generated; and, thus, (iii) that a co-targeting of both antigens via CAR T cells is feasible and promising.

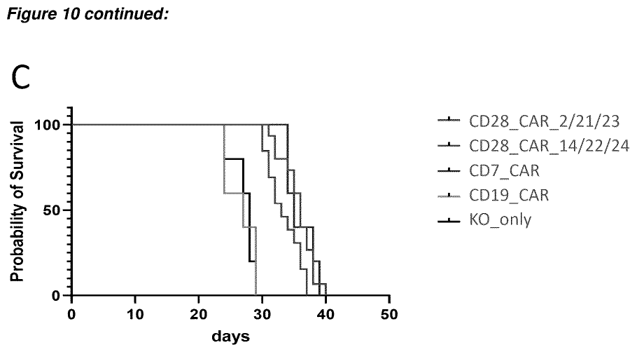

FIG. 10: Evaluation of CD28 CAR T cell in vivo functionality in a T-ALL mouse model. (A) Overall survival of NSG mice (bearing CCRF-CEM cells) assessed after single injection with different CAR T cell variants (cf. Table 2 in Example 7), i.e., six CD28 CAR T cell variants, the CD7 CAR T cell (positive control), or the CD19 CAR T cells or CD28 KO only T cells (negative controls). All single comparisons of each CD28 CAR and the CD7 CAR group against both negative controls show a significant improvement of survival (p<0.05). (B) In vivo imaging results demonstrate a significant reduction of leukemia load for all six tested CD28 CAR T cell variants vs. the negative controls (CD19 CAR T cells and CD28 KO only). (C) Survival data showing that CD28 CAR T cells bearing the TGN1412 scFv (CD28_CAR_2/21/23; cf. Table 2) yielded an even higher improvement in survival (p=0.029 G-B-Wilcoxon-test) than CAR T cells based on the CD28.3 scFv (CD28_CAR_14/22/24; cf. Table 2); see Example 7 and FIG. 10.

The examples illustrate the invention:

### Example 1: Expression of CD28 on T Cells of T-ALL Patients

Analysis of CD28 mRNA expression data from the Leukemia MILE Study (Haferlach et al., 2010) generated from the webtool bloodspot.eu²³ reveals that CD28 is highly expressed on the majority of T-lineage malignancies, including T lineage acute lymphoblastic leukemia and this overexpression is present on the majority of T-ALL patients (FIG. 1). CD28 overexpression was confirmed on primary pediatric T-ALL blasts and low expression on T-cell precursors in healthy bone marrow (FIG. 1B).

Compared to CD7, CD28 expression is lower in physiologic lymphoid precursor cells.

Therefore, targeting CD28 with CAR T cells would eliminate CD28 expressing cancer cells (T-lineage acute lymphoblastic leukemia/lymphoma and multiple myeloma) and some physiological T cells, especially mature T_{Helper} cells. Compared to CD7, CD28 expression is lower in physiologic lymphoid precursor cells.

### Example 2: Generation of CD28-Targeting CD28⁻ CAR T Cells and Functional Assessment Thereof

Materials and Methods:

Experimental Protocol for CRISPR/Cas-Mediated CD28-Knockout and CD28-CAR-Gene Delivery by Retroviral Transduction:
Day 1: T cells are activated (with CD3/CD28 stimulation, e.g., using TransAct reagent (Miltenyi Biotec) and IL5/IL17 in media).
Day 3: After 48 hours of in vitro expansion upon T cell stimulation, the following CRISPR/Cas9 knockout (KO) protocol for disrupting the endogenous CD28-encoding gene was performed:
   Heat up Thermoblock to 95° C.;
   Pre-warm 24-well plate and TexMACS™ medium (Miltenyi Biotec)+2.5% huAB serum;
   Thaw tracrRNA (“Alt-R® CRISPR-Cas9 tracrRNA” from Integrated DNA Technologies (IDT)) and crRNA (SEQ ID NO: 51); IDT), mix at a ratio of 1:1 (i.e., 9 μl tracrRNA+9 μl crRNA); and heat at 95° C. for 5 min, then cool to RT to allow formation of the (two-part) guideRNA (crRNA:tracrRNA);
   Take Cas9 nuclease (e.g., Alt-R® S.p. Cas9 Nuclease 3NLS (IDT)) and enhancer (e.g., Alt-R® Cas9 Electroporation Enhancer (IDT)) and bring to RT;
   Dilute Cas9 nuclease 1.5:1 with PBS (i.e., 15.6 μl Cas9 nuclease with 8.2 μl PBS (3.5 rct));
   For the formation of the ribonucleoprotein (RNP) complex (crRNA:tracrRNA:Cas9), add Cas9 working solution very slowly, moving the pipet tip in circles, into the gRNA solution;

| | CD28 KO (2.5x) | |
|---|---|---|
| gRNA | 18 | μl |
| Cas9 | 3.6 | μl |
| Enhancer | 1.3 | μl |
| RNP volume | 22.9 | μl |

Incubate mixture for 15 min at RT;
Resuspend 1×10⁶ cells/sample in 100 μl buffer M;
Pipette 10.4 μl RNPs into 96 well round bottom plate, add 100 μl cells and mix;
Transfer 100 μl cells with RNP into cuvette and electroporate with program T-023 (about 10 s);
Immediately add 250 μl warm medium and transfer cells into pre-warmed 24 well plate;
Wash cuvette with additional 250 μl warm medium, take 100 μl
→incubate 30 min 37° C.;
Add 500 μl/per well TexMACS medium+2.5% hu Ab serum
+20 ng/ml IL-7 (1:2000)+20 ng/ml IL-15 (1:2000);
Incubate for 96 hours 37° C.: count and add (go to 12 well if conc >1×10⁶/Ml);
Check phenotype 144 h after electroporation;
Check phenotype again prior to freezing (which takes place about 14 days after electroporation);
Day 4: Retronectin-coating of microwell-plates:
   The required amount of wells of a 24-well plate is coated with 2.5 μg of retronectin (5 μl retronectin+395 μl PBS per well); and the 24-well plate is then wrapped with parafilm and incubated overnight at 4° C. (alternatively for ≥2 h at 37° C.);
Day 5: Transduction:
   Retronectin/PBS solution is taken out of the wells (sucked off);
   Block the retronectin-coated wells with 500 μl blocking buffer (2% BSA in PBS; freshly prepared and filtered (0.2 μm filter)) per well; Incubate for 30 min at RT;
   Discard supernant, wash retronectin-coated wells with 1 ml washing buffer (1:40 dilution of 1 M HEPES with PBS (e.g., 19.5 ml PBS+0.5 ml HEPES) per well;
   Thaw CD28-CAR retrovirus containing supernatant (also referred to herein a “retrovirus supernatant”, “retroviral supernatant”, “virus supernatant” or “viral supernatant”), the preparation of which is conducted according to the protocol described herein below;
   Discard washing buffer (sucked off with pipette);
   Add 1 ml of the virus supernatant to each well of the retronectin-coated plate (in UT: only Medium (Dulbecco's Modified Eagle Medium (DMEM) 4+); centrifuge at 3000 g for 90 min at 32° C.; in the meantime (˜30 min before end of centrifugation): pool T cells in Falcon tube;
   Count the activated T cells (1×10⁶ per well/6×10⁶ in total);
   Centrifuge at 400 g for 5 min;
   Discard supernatant, resuspend cells to a concentration of 1×10⁶ cells/ml in TexMACS™ medium (Miltenyi Biotec)+2.5% human AB serum (HuAB)+12.5 ng/ml (0.5 μl/ml) IL-7 and IL-15 (without TransAct);
   Discard the virus supernatant and add 1×10⁶ (=1 ml) T cells (in 1 ml TexMACS™ medium+2.5% HuAB+12.5 ng/ml IL-7 and IL-15) to each well;
   Centrifuge at 450 g, 10 min, 32° C.;
   Incubate at 37° C.

Preparation of Retroviral Supernatant (as Used in the Above-Described Protocol):

Retroviral supernatant (comprising retroviral particles) as used for transduction of human T cells was generated according to the following protocol:

Genetic constructs are designed and cloned into a retroviral vector (retroviral expression plasmid “pMP71”; Addgene, cf. https://www.addgene.org/108214/) using a Gibson cloning kit available by New England Biolabs (NEB, https://international.neb.com/products/e5510-gibson-assembly-cloning-kit#Product%20 Information). Next, competent E. coli are transformed with the plasmid and grown on ampicillin-containing agar selection medium, resulting in selective growth of bacterial colonies containing the correct plasmid that includes an ampicillin resistance gene. Single colonies are selected, expanded and sequenced. If the desired sequence is correct, it can be used for the generation of virus supernatant.

To generate virus supernatant, first 293Vec-Galv cells (a HEK 293-based packaging cell line that produces retroviral vectors pseudotyped by the gibbon ape leukemia virus (GALV) envelope protein; https://www.biovecpharma.com/products.php?id=18) are transfected with plasmids containing the desired genetic insert coding a CD28 CAR molecule using the TransIT-293 reagent according to the manufacturers protocol (https://www.mirusbio.com/products/transfection/transit-293-transfection-reagent#product:2704). After 48 hours, the supernatant is harvested and used for transduction of 293Vec-RD114 cells (a HEK 293-based packaging cell line that produces retroviral vectors pseudotyped by the feline RD114 virus envelope protein; https://www.biovecpharma.com/products.php?id=19). These are expanded, transduction rate is determined by CD28 CAR molecule expression and 293Vec-RD114 cells are enriched for CD28 CAR molecule expressing cells in order to obtain high titer virus supernatant. Once they are enriched to more than 85% CD28 CAR molecule expressing cells, 5×10⁶ cells are seeded in a T75 bottle (surface 75 cm²), incubated for 72 hours, and subsequently the supernatant of the culture is filtered (0.45 μM) and either directly used for T cell transduction or frozen at −80° C. for later use.

The expression cassette includes a 5′ T7 promotor (underlined), the illustrated sequence represents the 100 bp upstream of the (ATG) start codon of pMP71:

(SEQ ID NO: 54)
tcgagtaatacgactcactatagggagacccaagctggctaggtaagct
tgatcaacaagtttgtacaaaaaagcaggctccgcggccgcccccttca
cc.

Results:

For generating CD28⁻ CAR T cells, CRISPR/Cas9-mediated knockout (KO) of CD28 was conducted in T cells from healthy donors to generate T cells that would be fratricide-resistant in a CD28⁻ CAR setup (FIGS. 2A&B). Next, obtained CD28^{negative} T cells were subjected to functional testing in order to assess their usability for adoptive T cell therapy. Surprisingly, it was found that CD28-knockout (CD28-KO) was stable over time, does not impair T cell expansion in vitro, and does not affect CD4/CD8 ratio (FIGS. 2 C&D). Moreover, T cell proliferation, interferon-gamma secretion, CD25 surface expression as activation marker and cytotoxicity in a blinatumomab (i.e., a bi-specific antibody which binds to CD3 on T cells and CD19 on B-cells) co-culture-assay was not impaired (FIG. 3). Only in a setup including CD28 stimulation (e.g., by CD3/CD28 co-stimulation), decreased proliferation and interferon-gamma secretion was observed (FIGS. 3 A&B). Then, 20 different retroviral constructs were designed having either a short (15 amino acids) or a long (55 amino acids) hinge region (as defined by SEQ ID NO: 6 and 5, respectively) between the scFv and the transmembrane domain; and two different sequential arrangements of the heavy and light chain variable domains (VH-linker-VL or VL-linker-VH) in their respective scFv, wherein the VH and VL domains of the scFvs are from several known antibodies against CD28²⁴⁻²⁷ (Table 1) and the linker is a “Whitlow linker” (SEQ ID NO: 2) as referred to herein above.

TABLE 1
Overview of 20 different chimeric antigen receptor constructs generated. Sequences
from five different monoclonal anti-human CD28 antibodies were used,
with either short or long hinge domain an either light-heavy
or heavy-light orientation of variable antibody chains.
Transmembrane (TM) and intracellular (IC) sequence
contained sequences from CD8 alpha,
CD28 and CD3-zeta. Constructs 1, 2, 11, 12, 14
were both expressed on T cells and eliminated
CD28⁺ target cell lines, constructs 5, 6, 8 were expressed
on T cells but did not kill CD28⁺ target cells and constructs
3, 4, 7, 9, 10, 15-20 were not expressed on T cells.

| Construct ID | scFv | Chain order | Hinge | TM | IC |
|---|---|---|---|---|---|
| CD28_CAR_1 | TGN1412 | light-heavy | short | CD8-TM | CD28_3z |
| CD28_CAR_2 | TGN1412 | light-heavy | long | CD8-TM | CD28_3z |
| CD28_CAR_3 | CD28.3 | light-heavy | short | CD8-TM | CD28_3z |
| CD28_CAR_4 | CD28.3 | light-heavy | long | CD8-TM | CD28_3z |
| CD28_CAR_5 | Cl1B4 | light-heavy | short | CD8-TM | CD28_3z |
| CD28_CAR_6 | Cl1B4 | light-heavy | long | CD8-TM | CD28_3z |
| CD28_CAR_7 | cl83 | light-heavy | short | CD8-TM | CD28_3z |
| CD28_CAR_8 | cl83 | light-heavy | long | CD8-TM | CD28_3z |
| CD28_CAR_9 | Fengfeng | light-heavy | short | CD8-TM | CD28_3z |
| CD28_CAR_10 | Fengfeng | light-heavy | long | CD8-TM | CD28_3z |
| CD28_CAR_11 | TGN1412 | heavy-light | short | CD8-TM | CD28_3z |
| CD28_CAR_12 | TGN1412 | heavy-light | long | CD8-TM | CD28_3z |
| CD28_CAR_13 | CD28.3 | heavy-light | short | CD8-TM | CD28_3z |
| CD28_CAR_14 | CD28.3 | heavy-light | long | CD8-TM | CD28_3z |
| CD28_CAR_15 | Cl1B4 | heavy-light | short | CD8-TM | CD28_3z |
| CD28_CAR_16 | Cl1B4 | heavy-light | long | CD8-TM | CD28_3z |
| CD28_CAR_17 | cl83 | heavy-light | short | CD8-TM | CD28_3z |
| CD28_CAR_18 | cl83 | heavy-light | long | CD8-TM | CD28_3z |
| CD28_CAR_19 | Fengfeng | heavy-light | short | CD8-TM | CD28_3z |
| CD28_CAR_20 | Fengfeng | heavy-light | long | CD8-TM | CD28_3z |

Transduction and subsequent expression of eight CD28-CAR molecules was observed with transduction rates averaging 70% 14 days after activation (FIG. 5A). The experimental setup equals the scheme illustrated in FIG. 4 without CRISPR/Cas9 knockout of CD28. Interestingly, reduced CD28 expression was observed in CD28^{WT} T cells when functional CD28-CAR molecules were transduced (FIG. 5B), explained by killing of CD28⁺ cells.

Next, the inventors assessed whether the expression of functional CD28-CAR molecules on CD28^{WT} T cells leads to fratricide, which would be indicated by a decreased expansion compared with CD28-KO T cells. The experimental set-up for this experiment is depicted in FIG. 4. Using this procedure, a reduced expansion of CD28-CAR CD28^{WT}T cells was observed when compared to CD28-CAR CD28^{KO} T cells, indicating fratricide of CD28-CAR expressing CD28^{WT} T cells and that simultaneous knockout of CD28 prevents fratricide (FIG. 4C).

Conclusion:

Here, the inventors successfully generated a novel CAR-T cell strategy with different molecular design and CD28 knockout. CD28 is highly expressed on T-lineage malignancies like T-ALL, T-NHL or multiple myeloma. In contrast to previous target antigens of anti-T-lineage CAR T cells (e.g., CD7), CD28 is expressed at a later stage of lymphopoiesis, suggesting that physiologic T-cell progenitors could be spared by CD28-CARs. Lack of CD28 is not associated with severe T-cell dysfunction, but rather with mild immunodeficiency. In addition, CD28 is a target that is functionally relevant for CD28⁺ malignancies. Finally, there is currently no clinically approved CAR T cell therapy for T-cell malignancies. These novel anti-CD28 CARs will offer the possibility to target relapsed/refractory T-lineage malignancies with CAR T cells in the future.

### Example 3: Engagement of CD28 on Target T Cells by CD28-Binding CAR-T Cells does not Lead to Activation of Target T Cells

TGN1412 is a well-known humanized CD28-binding full-length monoclonal antibody which has previously been reported to not only specifically bind CD28, but to also acting as a strong agonist (“superagonist”) of CD28, i.e., that is capable of activating T cells, in particular regulatory T cells, without the need of simultaneous T cell receptor (TCR)-mediated co-stimulation (Beyersdorf N et al., Immunotargets Ther. (2015); 4:111-22).

The present inventors assessed whether CAR T cells containing a scFv derived from TGN1412 (CD28_CAR_1, CD28_CAR_2, CD28_CAR_11 and CD28_CAR_12 (Table 1) having a scFv containing the same heavy and light chain variable domains (VH and VL domains) as the full-length TGN1412 antibody would exert any particular effects on wild-type (and thus predominantly CD28-expressing) T cells, as compared to
(i) CAR T cells containing a scFv which also specifically binds CD28, but which has no known (super-)agonistic activity (CD28_CAR_14 having the same VH and VL domains as the full-length monoclonal antibody “CD28.3” (Vanhove B et al., Blood. (2003); 102(2):564-70)); or
(ii) CAR T cells containing a CD19-binding scFv (“CD19_CAR”); or
(iii) untransduced T cells (=T cells not transduced with a CAR encoding gene).

The inventors performed an in vitro assay, wherein either different CAR T cells or untransduced T cells were co-cultured with wild-type T cells in order to assess whether wild-type T cells would be activated by co-culture with TGN1412-scFv containing anti-CD28 CAR T cells. The in vitro assay was conducted as follows:

After in vitro expansion for 14 days, including CRISPR/Cas9-mediated CD28 knockout at day 3 after activation, untransduced, wild-type T cells and CAR T cells containing different genetic constructs (CD28_CAR_1, CD28_CAR_2, CD28_CAR_11, CD28_CAR_12, CD28_CAR_14 (see Table 1), and CD19_CAR were cryopreserved in 10% DMSO. After thawing, resting overnight at 37° C. and 5% CO₂ and CD56 depletion, the following experiment was performed:

Untransduced T cells were stained with a labelling dye (cell trace violet, ® ThermoFisher) and subsequently 20.000 labelled, untransduced T cells were co-cultured with 20.000 non-labelled CD28 CAR T (mean transduction rate: 70%) cells for 24 hours. After co-culture period, intracellular interferon gamma (IFNγ) levels were measured after 2 hours of incubation with 10 μg/ml Brefeldin A (Sigma-Aldrich®, Merck KG) (i.e., “Golgi-stop treatment”).

The results, depicted in FIG. 6, indicate median fluorescence intensity of IFNy from three independent donors. As evident from FIG. 6, no increased IFNy levels were detected in the wild-type T cells 24 hours after co-culture with either of the different CAR T cells or the untransduced T cells.

### Example 4: Functionality of Invention Against Multiple Myeloma Cell Line

Background: CD28 expression has been described on both multiple myeloma cell lines and patient samples, where it was shown to correlate with disease progression.¹³ CD28 expression on multiple myeloma was shown to induce a pro-survival and immunosuppressive microenvironment, as well as chemotherapy resistance.^{4,28}

Methods: CD19 and CD28 expression of two multiple myeloma cell lines (RPMI 8226 and MM.1S) was determined by flow cytometry. Next, we transduced activated human T cells of healthy donors with both a CD19 CAR and two CD28 CAR constructs (using the CD28.3 scFv and the CD28 or 4-1BB costimulatory domain). In order to determine cytotoxic function of the generated CAR T cells, we co-cultured them with luciferase transduced MM.1S cells and analyzed MM.1S viability via luminescence after 48 h. In order to document the activation of CAR T cells by presence of target antigen, we performed intracellular cytokine staining (ICS) of T cells 24 h after co-culture with RPMI 8226 cells. All experiments were performed at least in triplicates. In order to eliminate a confounding bias, CD28 knockout via CRISPR/Cas9 was performed in all T cells presented in this example. CAR detection on T cell surface was performed via Myc-tag.

Results: CD28 CAR T cells detect CD28 expression on multiple myeloma cell lines and are superior to CD19 CAR T cells in killing multiple myeloma cell lines. Flow cytometric analysis shows that CD19 expression is scarce on both RPMI 8226 and MM.1S cells while both cell lines express CD28 (FIG. 7A). Transduction rates of CD19 and CD28 CAR T cells was above 30% in all samples used (FIG. 7B). CD28 CAR T cells show superior target killing compared to both untransduced T cells and CD19 CAR T cells (FIG. 7C). Intracellular cytokine staining of CAR T cells for TNFα and IFNy document specific detection of RPM18226 by CD28 CAR T cells, while no signal is present in absence of target cells or in CD19 CAR T cells (FIGS. 7D&E). In summary, these data demonstrate that CD28 CAR T cells detect CD28 expression on and are effective in killing multiple myeloma cell lines.

### Example 5: Alternative Methods of CD28 CAR T Cell Detection

Background: We initially used a short amino acid sequence attached to the CAR sequence derived from the human myc proto-oncogene in order to detect transduced CAR T cells. Two potentially conceivable disadvantages of using a Myc-tag led the inventors to seek alternative approaches: (a) depending on the position of the Myc tag within the CAR construct and the 3D structure of the CAR molecule, the epitope might not be well accessible for staining with an anti-Myc antibody; (b) although only consisting of a few amino acids, the origin of the Myc tag is a human proto-oncogene and this fact may have negative implications in view of a regulatory approval of clinical applications of the CD28 CAR T cells.

Methods: Consequently, the inventors explored two alternative methods: first, a polynucleotide sequence encoding truncated epidermal growth factor receptor (EGFRt) (SEQ ID NO: 55) was added into the expression cassette as second transgene after a polynucleotide sequence encoding T2A linker sequence (SEQ ID NO: 56). Secondly, a staining protocol was developed to directly stain the CD28 CAR molecule by adding recombinant CD28 protein with a poly-His-tag and then detecting this poly-His-tag with an anti-His antibody. All cells were subjected to CD28 knockout by CRISPR/Cas9.

Results: Alternative staining methods for CD28 CAR molecule detection on T cells. A EGFRt linker molecule was inserted into the expression cassette (FIG. 8A) similarly as described previously.^{29,30} A two-step staining protocol was developed: a T cell (1) expressing a CD28 CAR molecule (2) was first incubated for 30 min at room temperature with recombinant CD28 protein (3) with a poly-His tag (red dot). After a washing step, an anti-His-antibody (4) labelled with a fluorophore was added, thus facilitating tag-free CAR T cell detection (FIG. 8B). For some constructs, tag-free staining and EGFRt staining led to the same transduction rates, i.e. for the TGN1412 construct (FIG. 8C, left panel). However, in other constructs such as the CD28.3 construct presented here (middle panel: before and right panel: after target co-culture), only after target co-culture, the same transduction rates were observed by EGFRt expression and direct CAR staining. In summary, three different CAR staining protocols for CD28 CAR T cells are exemplified: some enable the use of monoclonal antibodies as a “safety switch” in a clinical setting (EGFRt) while others render the CAR design simpler which is beneficial in view of technical and regulatory perspectives.

### Example 6: Combination of CD28 CAR T Cells with CAR T Cells Specific for Alternative Antigens

Background: It seems possible that the success of CD19 single targeting in some B cell malignancies cannot be reproduced in other malignant entities. The inventors consider CD7 a promising target antigen in clinical development for T cell precursor childhood leukemia.³¹ Consequently, the inventors set out to compare the CD28 CAR T cells against CD7 CAR T cells and analyze the feasibility of co-targeting of CD7 and CD28 by CAR T cells.

Methods: A published sequence of a CD7 specific scFv³² was introduced into the CAR backbone. Next, the inventors transduced activated human T cells with two lead CD28 CAR constructs (based on the TGN1412 and the CD28.3 scFv) and compared their cytotoxic capacities in co-culture assays. Finally, knockout of CD7 and CD28 was performed by CRISPR/Cas9 in order to verify the feasibility of double knockout. The last step will be necessary, when CD7 and CD28 specific CAR T cells are administered at the same time in order to prevent fratricide in this setting.

Results: Comparison to and feasibility of co-targeting of CD7 CAR T cells to CD28 CAR T cells. Transduction rates of two CD28 CARs and of one CD7 CAR construct on physiologic T cells after 14 days of expansion in vitro (n=4) (FIG. 9A). Cytotoxicity of these three CAR T cell populations against Jurkat TCP-ALL cell line: at a E:T ratio of 0.04:1, CD28.3 CD28 CAR T cells show a higher cytotoxicity than both TGN1412 CD28 CAR T cells and CD7 CAR T cells, while no difference can be seen between all constructs tested at higher E:T ratios (FIG. 9B). Knockdown of CD7 and CD28 is feasible in primary T cells using CRISPR/Cas9 (FIG. 9C). Thus, these results show that CD28 CARs possess comparable cytotoxicity against TCP-ALL cell lines, and as CD7/CD28 double knockout T cells can be generated, a co-targeting of both antigens via CAR T cells seems feasible and promising.

### Example 7: Evaluation of CD28 CAR T Cell Functionality In Vivo

Generation of Six Different Sets of CD28 CAR T Cells

Six different sets of CD28 CAR T cells were generated from healthy donor derived primary T cells that were subjected to CD28 knockout prior to being retrovirally transfected with the different CD28 CAR molecules as indicated in the following Table 2:

TABLE 2
(Additional) CD28 CAR molecules. The constructs denoted “CD28_CAR_2”
and “CD28_CAR_14” correspond to those identically referred to in Table 1, above.
The constructs denoted “CD28_CAR_21” and “CD28_CAR_22” differ from the
constructs “CD28_CAR_2” “CD28_CAR_14”, respectively, with respect to their
intracellular (IC) portions, i.e., by bearing (besides a CD3-zeta (CD3ζ) signaling domain)
a 4-1BB signaling domain instead of a CD28 signaling domain.
The constructs denoted “CD28_CAR_23” and “CD28_CAR_24”
have an IC portion which comprises a CD28 signaling domain,
a 4-1BB signaling domain, and a CD3-zeta (CD3ζ) signaling domain.

| | | Chain | | | |
|---|---|---|---|---|---|
| Construct ID | scFv | order | Hinge | TM | IC |
| CD28_CAR_2 | TGN1412 | light-heavy | long | CD8-TM | CD28_3z |
| CD28_CAR_14 | CD28.3 | heavy-light | long | CD8-TM | CD28_3z |
| CD28_CAR_21 | TGN1412 | light-heavy | long | CD8-TM | 4-1BB_3z |
| CD28_CAR_22 | CD28.3 | heavy-light | long | CD8-TM | 4-1BB_3z |
| CD28_CAR_23 | TGN1412 | light-heavy | long | CD8-TM | CD28_4-1BB_3z |
| CD28_CAR_24 | CD28.3 | heavy-light | long | CD8-TM | CD28_4-1BB_3z |

In detail, the generated sets of CD28 CAR T cells express six different CAR molecules: CD28_CAR_2 and CD28_CAR_14 comprise in their ectodomain an anti-CD28 scFv (TGN1412 or CD28.2, respectively) and in their endodomain the CD28 and CD3-zeta (CD3ζ) signaling (stimulatory) domains; CD28_CAR_21 and CD28_CAR_22 comprise in their ectodomain an anti-CD28 scFv (TGN1412 or CD28.2, respectively) and in their endodomain the 4-1 BB and CD3-zeta (CD3ζ) signaling domains; and CD28_CAR_23 and CD28_CAR_24 comprise in their ectodomain an anti-CD28 scFv (TGN1412 or CD28.2, respectively) and in their endodomain (in N- to C-terminal order) the CD28, 4-1 BB and CD3-zeta (CD3) signaling domains.

Accordingly, CAR molecules derived from two different scFvs (TGN1412 or CD28.2, respectively) were used in two second-generation versions (i.e., CD28_CAR_2 and CD28_CAR_14, each comprising the CD28 and CD3 stimulatory domains, and CD28_CAR_21 and CD28_CAR_22, each comprising the 4-1 BB and CD3 stimulatory domains) and one third-generation version (i.e., CD28_CAR_23 and CD28_CAR_24 each comprising the CD28, CD3ζ, and 4-1 BB stimulatory domains).

Generation of CD7 CAR T Cells as Positive Control

CD7 CAR T cells were generated according to the protocol as published in Ref. 12 (Gomes-Silva D et al. Blood. 2017; 130(3):285-296) that is, CD7 CRISPR knockout was performed on day 2 after T cell activation and retroviral transduction was performed on day 4 after activation. CD7 CAR T cells showed specific and highly effective killing in vitro. These CD7 CAR T cells served as a positive control.

Generation of CD19 CAR T Cells as a Negative Control

CD19 CAR T cells were generated as a negative control. In order to control for potential side-effects of CD28 knockout, these CD19 CAR T cells were also subjected to CD28 knockout on day 2 after activation before CAR transduction was performed on day 4 after T cells activation.

Generation of CD28 Knockout (KO) Only T Cells as a Further Negative Control

CD28 knockout only T cells that did not harbor a CAR molecule were used as a further (second) negative control.

All nine conditions were generated from primary T cells derived from the same single donor and CAR T cells were cryopreserved in sufficient numbers 14 days after activation for further use.

Evaluation of CD28 CAR T Cells In Vivo Functionality in a Xenograft T-ALL Mouse Model

For assessing the in vivo functionality of the CD28 CAR T cells, 45 6-8 weeks old NSG mice (NOD-scid IL2Rgammanull; The Jackson Laboratory, Bar Harbour, ME, USA) were injected with 7.5e4 CCRF-CEM cells. These cells naturally express both CD7 and CD28 and were additionally transduced with GFP and firefly luciferase for in vivo imaging. Three days after CCRF-CEM injection, 2.5e6 CAR T cells were injected into mice (5 mice in each of the nine groups).

Subsequently, animals were scored on a daily basis and in vivo imaging was performed once a week.

Results:

FIG. 10A documents the specific improvement of overall survival of all six CAR T cell groups and the positive control (CD7 CAR T cells) against each of the negative controls (CD19 CAR T cells and CD28 KO only CAR T cells). All single comparisons of CD28 CAR and the CD7 CAR group against both negative controls yielded a significant improvement of survival (p<0.05). Additionally, a significant reduction of leukemia load could be documented by in vivo imaging (FIG. 10B, method as described in Ref. 33 (Ebinger et al., Cancer Cell. 2016; 30(6):849-862). Interestingly, CAR T cells bearing the TGN1412 scFv yielded the most pronounced improvement of survival, i.e., better than CAR T cells bearing the CD28.3 scFv (p=0.029 G-B-Wilcoxon-test, cf. FIG. 10C).

In summary, these results demonstrate the functionality of CD28 CAR T cells in vivo. Survival and leukemia imaging data revealed a comparable efficiency of CD28 CAR T cells and CD7 CAR T cells in the employed CCRF-CEM TCP-ALL mouse model. Finally, these findings show that CAR T cells bearing a TGN1412 scFv provided stronger improvement in survival as compared to CAR T cells bearing a CD28.3 scFv.

### FURTHER REFERENCES

1. Maciocia, P M, Wawrzyniecka, P A, Philip, B, Ricciardelli, I, Akarca, A U, Onuoha, S C, et al. (2017). Targeting the T cell receptor beta-chain constant region for immunotherapy of T cell malignancies. Nat Med 23: 1416-1423.
2. Gmyrek, G B, Pingel, J, Choi, J, and Green, J M (2017). Functional analysis of acquired CD28 mutations identified in cutaneous T cell lymphoma. Cell Immunol 319: 28-34.
3. Rohr, J, Guo, S, Huo, J, Bouska, A, Lachel, C, Li, Y, et al. (2016). Recurrent activating mutations of CD28 in peripheral T-cell lymphomas. Leukemia 30: 1062-1070.
4. Murray, M E, Gavile, C M, Nair, J R, Koorella, C, Carlson, L M, Buac, D, et al. (2014). CD28-mediated pro-survival signaling induces chemotherapeutic resistance in multiple myeloma. Blood 123: 3770-3779.
5. Rozanski, C H, Utley, A, Carlson, L M, Farren, M R, Murray, M, Russell, L M, et al. (2015). CD28 Promotes Plasma Cell Survival, Sustained Antibody Responses, and BLIMP-1 Upregulation through Its Distal PYAP Proline Motif. J Immunol 194: 4717-4728.
6. Sadelain, M (2015). CAR therapy: the CD19 paradigm. J Clin Invest 125: 3392-3400.
7. Lee, D W, Kochenderfer, J N, Stetler-Stevenson, M, Cui, Y K, Delbrook, C, Feldman, S A, et al. (2015). T cells expressing CD19 chimeric antigen receptors for acute lymphoblastic leukaemia in children and young adults: a phase 1 dose-escalation trial. Lancet (London, England) 385: 517-528.
8. Maude, S L, Laetsch, T W, Buechner, J, Rives, S, Boyer, M, Bittencourt, H, et al. (2018). Tisagenlecleucel in Children and Young Adults with B-Cell Lymphoblastic Leukemia. New England Journal of Medicine 378: 439-448.
9. Gardner, R A, Finney, O, Annesley, C, Brakke, H, Summers, C, Leger, K, et al. (2017). Intent-to-treat leukemia remission by CD19 CAR T cells of defined formulation and dose in children and young adults. Blood 129: 3322-3331.
10. O'Leary, M C, Lu, X, Huang, Y, Lin, X, Mahmood, I, Przepiorka, D, et al. (2018). FDA Approval Summary: Tisagenlecleucel for Treatment of Patients with Relapsed or Refractory B-cell Precursor Acute Lymphoblastic Leukemia. Clinical Cancer Research.
11. Mamonkin, M, Rouce, R H, Tashiro, H, and Brenner, MK (2015). A T-cell-directed chimeric antigen receptor for the selective treatment of T-cell malignancies. Blood 126: 983-992.
12. Gomes-Silva, D, Srinivasan, M, Sharma, S, Lee, C M, Wagner, D L, Davis, T H, et al. (2017). CD7-edited T cells expressing a CD7-specific CAR for the therapy of T-cell malignancies. Blood 130: 285-296.
13. Robillard, N, Jego, G, Pellat-Deceunynck, C, Pineau, D, Puthier, D, Mellerin, M P, et al. (1998). CD28, a marker associated with tumoral expansion in multiple myeloma. Clin Cancer Res 4: 1521-1526.
14. Shahinian, A, Pfeffer, K, Lee, K P, Kundig, T M, Kishihara, K, Wakeham, A, et al. (1993). Differential T cell costimulatory requirements in CD28-deficient mice. Science 261: 609-612.
15. Mestermann, K, Giavridis, T, Weber, J, Rydzek, J, Frenz, S, Nerreter, T, et al. (2019). The tyrosine kinase inhibitor dasatinib acts as a pharmacologic on/off switch for CAR T cells. Sci Transl Med 11.
16. Bonifant, C L, Jackson, H J, Brentjens, R J, and Curran, KJ (2016). Toxicity and management in CAR T-cell therapy. Mol Ther Oncolytics 3: 16011.
17. Gross, G, Waks, T, and Eshhar, Z (1989). Expression of immunoglobulin-T-cell receptor chimeric molecules as functional receptors with antibody-type specificity. Proc Natl Acad Sci USA 86: 10024-10028.
18. Fleischer, L C, Spencer, H T, and Raikar, SS (2019). Targeting T cell malignancies using CAR-based immunotherapy: challenges and potential solutions. J Hematol Oncol 12: 141.
19. Alcantara, M, Tesio, M, June, C H, and Houot, R (2018). CAR T-cells for T-cell malignancies: challenges in distinguishing between therapeutic, normal, and neoplastic T-cells. Leukemia 32: 2307-2315.
20. Cooper, M L, and DiPersio, J F (2019). Chimeric antigen receptor T cells (CAR-T) for the treatment of T-cell malignancies. Best Pract Res Clin Haematol 32: 101097.
21. Haynes, B F, Martin, M E, Kay, H H, and Kurtzberg, J (1988). Early events in human T cell ontogeny. Phenotypic characterization and immunohistologic localization of T cell precursors in early human fetal tissues. J Exp Med 168: 1061-1080.
22. Majzner, R G, and Mackall, CL (2018). Tumor Antigen Escape from CAR T-cell Therapy. Cancer Discov 8: 1219-1226.
23. Bagger, F O, Kinalis, S, and Rapin, N (2019). BloodSpot: a database of healthy and malignant haematopoiesis updated with purified and single cell mRNA sequencing profiles. Nucleic Acids Res 47: D881-D885.
24. Hunig, T (2007). Manipulation of regulatory T-cell number and function with CD28-specific monoclonal antibodies. Adv Immunol 95:111-148.
25. Zheng, F, Qiu, Y, Chen, Y, Chen, P, Zhu, Y, Xie, W, et al. (2009). Cloning, purification and bioactivity assay of human CD28 single-chain antibody in Escherichia coli. Cytotechnology 60: 85-94.
26. Rothe, A, Nathanielsz, A, Hosse, R J, Oberhauser, F, Strandmann, E P, Engert, A, et al. (2007). Selection of human anti-CD28 scFvs from a T-NHL related scFv library using ribosome display. J Biotechnol 130: 448-454.
27. Vanhove, B, Laflamme, G, Coulon, F, Mougin, M, Vusio, P, Haspot, F, et al. (2003). Selective blockade of CD28 and not CTLA-4 with a single-chain Fv-alpha1-antitrypsin fusion antibody. Blood 102: 564-570.
28. Nair J R, Carlson L M, Koorella C, et al. CD28 Expressed on Malignant Plasma Cells Induces a Prosurvival and Immunosuppressive Microenvironment. The Journal of Immunology (2011); 187(3):1243-1253.
29. Liu Z, Chen O, Wall J B J, et al. Systematic comparison of 2A peptides for cloning multi-genes in a polycistronic vector. Sci Rep (2017); 7(1):2193.
30. Wang X, Chang W C, Wong C W, Colcher D, Sherman M, Ostberg J R, Forman S J, Riddell S R, Jensen M C. A transgene-encoded cell surface polypeptide for selection, in vivo tracking, and ablation of engineered cells. Blood. (2011)4; 118(5):1255-63.
31. Pan J, Tan Y, Wang G, et al. Donor-Derived CD7 Chimeric Antigen Receptor T Cells for T-Cell Acute Lymphoblastic Leukemia: First-in-Human, Phase I Trial. JCO (2021); 39(30):3340-3351.
32. Pauza M E, Doumbia S O, Pennell C A. Construction and characterization of human CD7-specific single-chain Fv immunotoxins. J Immunol 1997; 158(7):3259-3269.
33. Ebinger S, Özdemir E Z, Ziegenhain C, et al. Characterization of Rare, Dormant, and Therapy-Resistant Cells in Acute Lymphoblastic Leukemia. Cancer Cell. 2016; 30(6):849-862.

### Example 8: Examples of Certain Embodiments

Listed hereafter are non-limiting examples of certain embodiments of the technology.
A1. A modified T cell, comprising:
   (a) a disrupted endogenous CD28-encoding gene; and
   (b) a polynucleotide encoding a chimeric antigen receptor (CAR), wherein the CAR comprises in its ectodomain at least one antigen binding moiety that is capable of specific binding to the extracellular portion of CD28.
A2. The modified T cell of embodiment 1, wherein the antigen binding moiety that is capable of specific binding to the extracellular portion of CD28 is an anti-CD28 antibody, preferably an anti-CD28 single-chain variable fragment (scFv);
   wherein preferably the anti-CD28 antibody or anti-CD28 scFv comprises:
   (a) a VH CDR1, CDR2 and CDR3 consisting of the amino acid sequences of SEQ ID NO: 8, 9 and 10, and a VL CDR1, CDR2 and CDR3 of the amino acid sequences of SEQ ID NO: 11, 12 and 13; or
   (b) a VH CDR1, CDR2 and CDR3 consisting of the amino acid sequences of SEQ ID NO: 14, 15 and 16, and a VL CDR1, CDR2 and CDR3 of the amino acid sequences of SEQ ID NO: 17, 18 and 19.
A3. The modified T cell of embodiment 1 or 2, wherein the CAR further comprises an endodomain comprising one or more T-cell-stimulatory molecules;
   wherein the T-cell-stimulatory molecule is preferably a signaling domain from a T-cell-co-stimulatory receptor, an immunoreceptor tyrosine-based activation motif (ITAM), and/or a Toll/interleukin-1 receptor (TIR) domain;
   wherein preferably
   (i) the T-cell-co-stimulatory receptor is selected from: CD28, ICOS (CD278), CD27, 4-1 BB (CD137, TNFRSF9), OX40 (CD134), CD27, IL-2R3, IL-15R-α, CD40L (CD154) and/or MyD88; and/or
   (ii) the ITAM is selected from: CD3-zeta (CD3ζ), DAP12, Fc-epsilon receptor 1 gamma chain, CD3-gamma, CD3-delta, CD3-epsilon, and CD79A (antigen receptor complex-associated protein alpha chain); and/or
   (iii) the TIR domain is the TIR domain of Toll-like receptor 2 (TRL2).
A4. The modified T cell of embodiment 3, wherein the endodomain of the CAR comprises a CD28 signaling domain and a CD3-zeta (CD3ζ) signaling domain.
A5. The modified T cell of any one of embodiments 1 to 4, wherein the CAR further comprises a transmembrane domain;
   wherein preferably the transmembrane domain comprises or consists of a transmembrane domain of a protein selected from the group of: a subunit of the T-cell receptor, CD3, CD4, CD7, CD8, CD27, CD28, OX40 (CD134), ICOS (CD278), PD-1 (CD279) and DAP12, more preferable from CD3-zeta (CD3ζ), CD4, CD8, or CD28; even more preferable from CD8.
A6. The modified T cell of any one of embodiments 1 to 5, wherein the disruption of the endogenous CD28-encoding gene is due to one or more nucleotide base insertions and/or deletions (‘InDels’) resulting from non-homologous end joining (NHEJ) DNA repair of DNA double-strand breaks (DSBs); wherein the DSBs are preferably resulting from a nuclease-based gene editing with a zinc finger nuclease (ZFN), transcription activator-like effector nuclease (TALEN), and/or Clustered Regularly Interspaced Short Palindromic Repeats (CRISPR)/Cas-based RNA-guided DNA endonuclease; and/or
   wherein the CAR-encoding polynucleotide is preferably integrated into the genome of the T cell, preferably by ex vivo retrovirus-based gene delivery.
A7. A population of modified T cells, comprising the modified T cell of any one of embodiments 1 to 6, wherein:
   (a) at least 25%, at least 50%, or at least 70% of the modified T cells of the population express the CAR on their surface;
   (b) at least 25%, at least 50%, or at least 70% of the modified T cells of the population express the CAR following at least 5 days, at least 7 days, or at least 10 days of in vitro proliferation; and/or
   (c) at least 50%, at least 60%, at least 70%, at least 80%, or at least 90% of the modified T cells of the population do not express a detectable level of CD28 protein; and/or
   (d) the population, when co-cultured in vitro with a population of non-modified T cells that express CD28, induces cell lysis of the non-modified T cells in the culture, wherein the initial ratio of modified to non-modified T cells is about equal; and/or
   (e) the modified T cells in the population have an in vitro clonal expansion rate of at least 30% per day.
A8. A method for generating modified T cells in vitro, comprising:
   (a) disrupting the endogenous CD28-encoding gene in T cells; and
   (b) introducing into said T cells a polynucleotide encoding a chimeric antigen receptor (CAR), wherein the CAR comprises in its ectodomain at least one antigen binding moiety that is capable of specific binding to the extracellular portion of CD28.
A9. Modified T cells obtained by the method of embodiment 8.
A10. Modified T cells of any one of embodiments 1 to 7 and 9 for use as a medicament.
A11. The modified T cells according to any one of embodiments 1 to 7 and 9 for use in treating a T cell-mediated disorder or other disorder which will benefit from an elimination of CD28-expressing-cells; preferably selected from
   (a) a T-cell hyperproliferative disorder; and/or
   (b) T-cell lymphoma (TCL), T-cell non-Hodgkin lymphoma (T-NHL), mycosis fungoides, anaplastic large cell lymphoma (ALCL), cutaneous T-cell lymphoma (CTCL), peripheral T-cell lymphoma (PTCL), precursor T-lymphoblastic lymphoma (Pre-T-LBL), T-cell acute lymphoblastic lymphoma (T-LBL), and/or angioimmunoblastic T cell lymphoma (AITL); and/or
   (c) T-cell leukemia (TLL), acute myeloid leukemia (AML), T-cell acute lymphoblastic leukemia (T-ALL), pediatric T-ALL, adult T-ALL, T-cell prolymphocytic leukemia (T-PLL), T-cell large granular lymphocyte (T-LGL) leukemia, and/or adult T cell lymphoma-leukemia (ATL); and/or
   (d) a T-cell-mediated autoimmune disease; and/or
   (e) Non-Hodgkin Lymphoma (NHL); and/or
   (f) a B-cell hyperproliferative disorder, preferably multiple myeloma (MM); and/or
   (g) any other disorder characterized by CD28-expressing disease-promoting cells.
A12. The modified T cells according to any one of embodiments 1 to 7 and 9 for use according to embodiment 11, wherein the disorder is mediated by T cells and/or B cells that are CD28⁺; and wherein optionally
   (a) said T cells are CD2⁻, CD5⁻, CD7⁻, CD30⁻, CD37⁻, and/or CCR4⁻; and/or
   (b) said B cells are CD19⁻; and/or
   (c) said T cells and/or said B cells are resistant to anti-X CAR T cell immunotherapy, wherein X is a cell surface antigen distinct from CD28; and/or
   (d) said T cells and/or said B cells are resistant to treatment with one or more chemotherapeutics.
A13. The modified T cells according to any one of embodiments 1 to 7 and 9 for use according to any one of embodiments 10 to 12, wherein the modified T cells are:
   (a) to be co-administered with:
      (i) an anti-X antibody or antibody-drug-conjugate, wherein X is a surface antigen distinct from CD28; wherein X is preferably CD2, CD5, CD7, CD28, CD30, CD37 or CCR4; and/or
      (ii) an anti-X CAR T cell immunotherapy, wherein X is a surface antigen distinct from CD28; wherein X is preferably CD2, CD5, CD7, CD28, CD30, CD37 or CCR4; and/or
      (iii) one or more inhibitors of T-cell inhibitory signaling, preferably an anti-PD-1 antibody and/or anti-CTLA4 antibody; and/or
   (b) to be administered prior to or after:
      (i) a chemotherapeutic treatment, wherein the chemotherapeutic is preferably one or more of cyclophosphamide, doxorubicin (Adriamycin), vincristine, L-asparaginase, methotrexate, prednisone, and/or cytarabine (ara-C); and/or
      (ii) stem cell transplantation, preferably after chemotherapeutic treatment and prior to stem cell transplantation.
A14. The modified T cell according to any one of embodiments 1 to 6, or the population of modified T cells of embodiment 7, or the modified T cells of embodiment 9 for use according to any one of embodiments 10 to 13, wherein the subject will benefit from a selective depletion of CD28-expressing cells, preferably CD28-expressing T-cells and/or CD28-expressing B-cells.
A15. Use of the modified T cell according to any one of embodiments 1 to 6, or of the population of modified T cells of embodiment 7, or of the modified T cells of embodiment 9, for selective depletion of CD28⁺ cells in a sample in vitro.

1. A modified T cell, comprising
(a) a disrupted endogenous CD28-encoding gene; and
(b) a polynucleotide encoding a chimeric antigen receptor (CAR), wherein the CAR comprises in its ectodomain at least one antigen binding moiety that is capable of specific binding to the extracellular portion of CD28.

2. The modified T cell of claim 1, wherein the antigen binding moiety that is capable of specific binding to the extracellular portion of CD28 is an anti-CD28 antibody, preferably an anti-CD28 single-chain variable fragment (scFv);
wherein preferably the anti-CD28 antibody or anti-CD28 scFv comprises:
(a) a VH CDR1, CDR2 and CDR3 consisting of the amino acid sequences of SEQ ID NO: 8, 9 and 10, and a VL CDR1, CDR2 and CDR3 of the amino acid sequences of SEQ ID NO: 11, 12 and 13; or
(b) a VH CDR1, CDR2 and CDR3 consisting of the amino acid sequences of SEQ ID NO: 14, 15 and 16, and a VL CDR1, CDR2 and CDR3 of the amino acid sequences of SEQ ID NO: 17, 18 and 19.

3. The modified T cell of claim 1, wherein the CAR further comprises an endodomain comprising one or more T-cell-stimulatory molecules;
wherein the T-cell-stimulatory molecule is preferably a signaling domain from a T-cell-co-stimulatory receptor, an immunoreceptor tyrosine-based activation motif (ITAM), and/or a Toll/interleukin-1 receptor (TIR) domain;
wherein preferably
(i) the T-cell-co-stimulatory receptor is selected from: CD28, ICOS (CD278), CD27, 4-1 BB (CD137, TNFRSF9), OX40 (CD134), CD27, IL-2R3, IL-15R-α, CD40L (CD154) and/or MyD88; and/or
(ii) the ITAM is selected from: CD3-zeta (CD3ζ), DAP12, Fc-epsilon receptor 1 gamma chain, CD3-gamma, CD3-delta, CD3-epsilon, and CD79A (antigen receptor complex-associated protein alpha chain); and/or
(iii) the TIR domain is the TIR domain of Toll-like receptor 2 (TRL2).

4. The modified T cell of claim 3, wherein the endodomain of the CAR comprises:
(i) a CD28 signaling domain and a CD3-zeta (CD3ζ) signaling domain; and/or
(ii) a 4-1 BB signaling domain and a CD3-zeta (CD3ζ) signaling domain; and/or
(iii) a CD28 signaling domain, a 4-1 BB signaling domain, and a CD3-zeta (CD3ζ). signaling domain.

5. The modified T cell of claim 1, wherein the CAR further comprises a transmembrane domain; wherein preferably the transmembrane domain comprises or consists of a transmembrane domain of a protein selected from the group of: a subunit of the T-cell receptor, CD3, CD4, CD7, CD8, CD27, CD28, OX40 (CD134), ICOS (CD278), PD-1 (CD279) and DAP12, more preferable from CD3-zeta (CD3ζ), CD4, CD8, or CD28; even more preferable from CD8.

6. The modified T cell of claim 1, wherein the disruption of the endogenous CD28-encoding gene is due to one or more nucleotide base insertions and/or deletions (‘InDels’) resulting from non-homologous end joining (NHEJ) DNA repair of DNA double-strand breaks (DSBs);
wherein the DSBs are preferably resulting from a nuclease-based gene editing with a zinc finger nuclease (ZFN), transcription activator-like effector nuclease (TALEN), and/or Clustered Regularly Interspaced Short Palindromic Repeats (CRISPR)/Cas-based RNA-guided DNA endonuclease; and/or
wherein the CAR-encoding polynucleotide is preferably integrated into the genome of the T cell, preferably by ex vivo retrovirus-based gene delivery.

7. A population of modified T cells, comprising the modified T cell of claim 1, wherein
(a) at least 25%, at least 50%, or at least 70% of the modified T cells of the population express the CAR on their surface;
(b) at least 25%, at least 50%, or at least 70% of the modified T cells of the population express the CAR following at least 5 days, at least 7 days, or at least 10 days of in vitro proliferation; and/or
(c) at least 50%, at least 60%, at least 70%, at least 80%, or at least 90% of the modified T cells of the population do not express a detectable level of CD28 protein; and/or
(d) the population, when co-cultured in vitro with a population of non-modified T cells that express CD28, induces cell lysis of the non-modified T cells in the culture, wherein the initial ratio of modified to non-modified T cells is about equal; and/or
(e) the modified T cells in the population have an in vitro clonal expansion rate of at least 30% per day.

8. A method for generating modified T cells in vitro, comprising
(a) disrupting the endogenous CD28-encoding gene in T cells; and
(b) introducing into said T cells a polynucleotide encoding a chimeric antigen receptor (CAR), wherein the CAR comprises in its ectodomain at least one antigen binding moiety that is capable of specific binding to the extracellular portion of CD28.

9. Modified T cells obtained by the method of claim 8.

10. A method for treating, delaying the progression of, and/or otherwise ameliorating a symptom of a disorder in a subject in need thereof, the method comprising administering to the subject modified T cells according to claim 1 for use as a medicament.

11. A method for treating, delaying the progression of, and/or otherwise ameliorating a symptom of a T cell-mediated disorder, or other disorder which will benefit from an elimination of CD28-expressing-cells, in a subject in need thereof, the method comprising administering to the subject modified T cells according to claim 1, wherein said T cell-mediated disorder or other disorder is preferably selected from:
(a) a T-cell hyperproliferative disorder; and/or
(b) T-cell lymphoma (TCL), T-cell non-Hodgkin lymphoma (T-NHL), mycosis fungoides, anaplastic large cell lymphoma (ALCL), cutaneous T-cell lymphoma (CTCL), peripheral T-cell lymphoma (PTCL), precursor T-lymphoblastic lymphoma (Pre-T-LBL), T-cell acute lymphoblastic lymphoma (T-LBL), and/or angioimmunoblastic T cell lymphoma (AITL); and/or
(c) T-cell leukemia (TLL), acute myeloid leukemia (AML), T-cell acute lymphoblastic leukemia (T-ALL), pediatric T-ALL, adult T-ALL, T-cell prolymphocytic leukemia (T-PLL), T-cell large granular lymphocyte (T-LGL) leukemia, and/or adult T cell lymphoma-leukemia (ATL); and/or
(d) a T-cell-mediated autoimmune disease; and/or
(e) Non-Hodgkin Lymphoma (NHL); and/or
(f) a B-cell hyperproliferative disorder, preferably multiple myeloma (MM); and/or
(g) any other disorder characterized by CD28-expressing disease-promoting cells.

12. The method of claim 11, wherein said disorder is mediated by T cells and/or B cells that are CD28⁺;
and wherein optionally
(a) said T cells are CD2⁻, CD5⁻, CD7⁻, CD30⁻, CD37⁻, and/or CCR4⁻; and/or
(b) said B cells are CD19⁻; and/or
(c) said T cells and/or said B cells are resistant to anti-X CAR T cell immunotherapy, wherein X is a cell surface antigen distinct from CD28; and/or
(d) said T cells and/or said B cells are resistant to treatment with one or more chemotherapeutics.

13. The method of claim 11, wherein the modified T cells are
(a) co-administered with:
(i) an anti-X antibody or antibody-drug-conjugate, wherein X is a surface antigen distinct from CD28; wherein X is preferably CD2, CD5, CD7, CD28, CD30, CD37 or CCR4; and/or
(ii) an anti-X CAR T cell immunotherapy, wherein X is a surface antigen distinct from CD28; wherein X is preferably CD2, CD5, CD7, CD28, CD30, CD37 or CCR4; and/or
(iii) one or more inhibitors of T-cell inhibitory signaling, preferably an anti-PD-1 antibody and/or anti-CTLA4 antibody; and/or
(b) administered prior to or after:
(i) a chemotherapeutic treatment, wherein the chemotherapeutic is preferably one or more of cyclophosphamide, doxorubicin (Adriamycin), vincristine, L-asparaginase, methotrexate, prednisone, and/or cytarabine (ara-C); and/or
(ii) stem cell transplantation, preferably after chemotherapeutic treatment and prior to stem cell transplantation.

14. The method of claim 11, wherein the subject will benefit from a selective depletion of CD28-expressing cells, preferably CD28-expressing T-cells and/or CD28-expressing B-cells.

15. Use of the modified T cell according of claim 1 for selective depletion of CD28⁺ cells in a sample in vitro.


SEQUENCE LISTING (71 sequences)

SEQ ID NO: 1 - protein, 5 aa, synthetic construct
  REGION 1..5: note = possible sequence unit of hinge or linker (GGGGS)n
GGGGS

SEQ ID NO: 2 - protein, 18 aa, synthetic construct
  REGION 1..18: note = Whitlow linker
GSTSGSGKPGSGEGSTKG

SEQ ID NO: 3 - protein, 21 aa, Homo sapiens
  REGION 1..21: note = human CD8 alpha signal peptide
MALPVTALLLPLALLLHAARP

SEQ ID NO: 4 - protein, 22 aa, Homo sapiens
  REGION 1..22: note = signal peptide of human Colony Stimulating Factor 2 Receptor Subunit Alpha (CSF2RA)
MLLLVTSLLLCELPHPAFLLIP

SEQ ID NO: 5 - protein, 55 aa, Homo sapiens
  REGION 1..55: note = exemplary long hinge (human CD8 alpha extracellular portion)
FVPVFLPAKPTTTPAPRPPTPAPTIASQPLSLRPEACRPAAGGAVHTRGLDFACD

SEQ ID NO: 6 - protein, 15 aa, synthetic construct
  REGION 1..15: note = exemplary short hinge: (GGGGS)n, n = 3
GGGGSGGGGSGGGGS

SEQ ID NO: 7 - protein, 10 aa, synthetic construct
  REGION 1..10: note = myc-tag
EQKLISEEDL

SEQ ID NO: 8 - protein, 5 aa, synthetic construct
  REGION 1..5: note = monoclonal antibody TGN1412 - VH CDR1
SYYIH

SEQ ID NO: 9 - protein, 17 aa, synthetic construct
  REGION 1..17: note = monoclonal antibody TGN1412 - VH CDR2
CIYPGNVNTNYNEKFKD

SEQ ID NO: 10 - protein, 11 aa, synthetic construct
  REGION 1..11: note = monoclonal antibody TGN1412 - VH CDR3
SHYGLDWNFDV

SEQ ID NO: 11 - protein, 11 aa, synthetic construct
  REGION 1..11: note = monoclonal antibody TGN1412 - VL CDR1
HASQNIYVWLN

SEQ ID NO: 12 - protein, 7 aa, synthetic construct
  REGION 1..7: note = monoclonal antibody TGN1412 - VL CDR2
KASNLHT

SEQ ID NO: 13 - protein, 9 aa, synthetic construct
  REGION 1..9: note = monoclonal antibody TGN1412 - VL CDR3
QQGQTYPYT

SEQ ID NO: 14 - protein, 5 aa, synthetic construct
  REGION 1..5: note = monoclonal antibody CD28.3 - VH CDR1
EYIIH

SEQ ID NO: 15 - protein, 17 aa, synthetic construct
  REGION 1..17: note = monoclonal antibody CD28.3 - VH CDR2
WFYPGSNDIQYNAKFKG

SEQ ID NO: 16 - protein, 12 aa, synthetic construct
  REGION 1..12: note = monoclonal antibody CD28.3 - VH CDR3
RDDFSGYDALPY

SEQ ID NO: 17 - protein, 11 aa, synthetic construct
  REGION 1..11: note = monoclonal antibody CD28.3 - VL CDR1
RTNENIYSNLA

SEQ ID NO: 18 - protein, 7 aa, synthetic construct
  REGION 1..7: note = monoclonal antibody CD28.3 - VL CDR2
AATHLVE

SEQ ID NO: 19 - protein, 9 aa, synthetic construct
  REGION 1..9: note = monoclonal antibody CD28.3 - VL CDR3
QHFWGTPCT

SEQ ID NO: 20 - protein, 120 aa, synthetic construct
  REGION 1..120: note = monoclonal antibody TGN1412 VH
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 21 - protein, 110 aa, synthetic construct
  REGION 1..110: note = monoclonal antibody TGN1412 VL
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTV

SEQ ID NO: 22 - protein, 120 aa, synthetic construct
  REGION 1..120: note = monoclonal antibody CD28.3 - VH
VKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSS

SEQ ID NO: 23 - protein, 108 aa, synthetic construct
  REGION 1..108: note = monoclonal antibody CD28.3 - VL
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKR

SEQ ID NO: 24 - protein, 248 aa, synthetic construct
  REGION 1..248: note = scFv of construct CD28_CAR_2 (without signal peptide)
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVGSTSGSGKPGSGEGSTKGQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 25 - protein, 248 aa, synthetic construct
  REGION 1..248: note = scFv of construct CD28_CAR_12 (without signal peptide)
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGSTSGSGKPGSGEGSTKGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTV

SEQ ID NO: 26 - protein, 246 aa, synthetic construct
  REGION 1..246: note = scFv of construct CD28_CAR_14 (without signal peptide)
VKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSGSTSGSGKPGSGEGSTKGDIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKR

SEQ ID NO: 27 - protein, 41 aa, Homo sapiens
  REGION 1..41: note = human CD28 signaling domain
RSKRSRLLHSDYMNMTPRRPGPTRKHYQPYAPPRDFAAYRS

SEQ ID NO: 28 - protein, 41 aa, Mus musculus
  REGION 1..41: note = murine CD28 signaling domain
NSRRNRLLQSDYMNMTPRRPGLTRKPYQPYAPARDFAAYRP

SEQ ID NO: 29 - protein, 41 aa, Rattus norvegicus
  REGION 1..41: note = rat CD28 signaling domain
NSRRNRLLQSDYMNMTPRRLGPTRKHYQPYAPARDFAAYRP

SEQ ID NO: 30 - protein, 112 aa, Homo sapiens
  REGION 1..112: note = human CD3 signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 143 aa, Mus musculus
  REGION 1..143: note = murine CD3 signaling domain
QSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 32 - protein, 143 aa, Rattus norvegicus
  REGION 1..143: note = rat CD3 signaling domain
QSFGLLDPKLCYMLDGILFIYGVIVTALYLRAKFSRSADAAAYLQDPNQLYNELNLGRREEYDVLDKKRPRDPEMGGKQQRRRNPQEGVYNALQKDKMAEAYSEIGMKGERRRGKGHDGLYQGLSTATKDTYDALHMQTLPPR

SEQ ID NO: 33 - protein, 21 aa, Homo sapiens
  REGION 1..21: note = human CD8 alpha transmembrane domain
IYIWAPLAGTCGVLLLSLVIT

SEQ ID NO: 34 - protein, 21 aa, Mus musculus
  REGION 1..21: note = murine CD8 alpha transmembrane domain
IWAPLAGICVALLLSLIITLI

SEQ ID NO: 35 - protein, 21 aa, Rattus norvegicus
  REGION 1..21: note = rat CD8 alpha transmembrane domain
IWAPLAGICAVLLLSLVITLI

SEQ ID NO: 36 - protein, 7 aa, Homo sapiens
  REGION 1..7: note = human CD8 alpha intracellular peptide
LYCNHRN

SEQ ID NO: 37 - protein, 7 aa, Mus musculus
  REGION 1..7: note = murine CD8 alpha intracellular peptide
CYHRSRK

SEQ ID NO: 38 - protein, 7 aa, Rattus norvegicus
  REGION 1..7: note = rat CD8 alpha intracellular peptide
CCHRNRR

SEQ ID NO: 39 - protein, 181 aa, synthetic construct
  REGION 1..181: note = hCD8 alpha TM +intracell. peptide, hCD28 signaling domain, hCD3 signaling domain
IYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 476 aa, synthetic construct
  REGION 1..476: note = CD28_CAR_1 (including signal peptide)
MLLLVTSLLLCELPHPAFLLIPDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVGSTSGSGKPGSGEGSTKGQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSEQKLISEEDLGGGGSGGGGSGGGGSIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 516 aa, synthetic construct
  REGION 1..516: note = CD28_CAR_2 (including signal peptide)
MLLLVTSLLLCELPHPAFLLIPDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVGSTSGSGKPGSGEGSTKGQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSEQKLISEEDLFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 476 aa, synthetic construct
  REGION 1..476: note = CD28_CAR_11 (including signal peptide)
MLLLVTSLLLCELPHPAFLLIPQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGSTSGSGKPGSGEGSTKGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVEQKLISEEDLGGGGSGGGGSGGGGSIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 516 aa, synthetic construct
  REGION 1..516: note = CD28_CAR_12 (including signal peptide)
MLLLVTSLLLCELPHPAFLLIPQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGSTSGSGKPGSGEGSTKGDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVEQKLISEEDLFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 514 aa, synthetic construct
  REGION 1..514: note = CD28_CAR_14 (including signal peptide)
MLLLVTSLLLCELPHPAFLLIPVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSGSTSGSGKPGSGEGSTKGDIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKREQKLISEEDLFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 1431 bp, synthetic construct
  misc_feature 1..1431: note = CD28_CAR_1 encoding nucleotide sequence
  source 1..1431: mol_type = other DNA
atgttgctgttagttacatccttgctcctgtgcgaattgccacatccagcattccttttgatccccgatatccaaatgacacaaagcccgagttccctctctgccagcgtaggagatcgcgtaactataacttgccacgctagccaaaatatttacgtatggctgaactggtatcagcagaaaccaggaaaagcaccgaaacttctcatttataaagccagcaatttgcacacaggggttccgtcccgcttttccgggtcaggctcaggtacggacttcaccctgacgatttccagccttcaacccgaggactttgccacttattattgtcaacagggtcaaacatacccctataccttcggcggtgggactaaagtcgaaattaagaggaccgttggcagtactagcggctccggcaaaccagggtccggcgaaggatccaccaagggacaagttcagctggtacaaagtggcgcagaggtaaagaaacctggcgccagcgtgaaggtttcctgtaaggcatccggatacacctttacaagctactatattcattgggttcggcaagctcctggccaaggtttggaatggattgggtgcatttaccccggtaatgtcaacaccaattacaacgaaaagtttaaggaccgggccacgttgactgttgatacgtctatctccacagcgtacatggagctctccagacttagatctgatgatacagcagtgtatttctgtacgagatctcattatggtcttgactggaactttgacgtctggggacagggaacgaccgtcactgtttctagtgaacagaaactgatctccgaggaagatctcggaggaggcgggtcaggtggggggggttctggggggggcgggtcaatatatatatgggcaccactggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 46 - DNA, 1551 bp, synthetic construct
  misc_feature 1..1551: note = CD28_CAR_2 encoding nucleotide sequence
  source 1..1551: mol_type = other DNA
atgttgctgttagttacatccttgctcctgtgcgaactcccacatcccgcttttctcctgatcccagatattcaaatgacccaaagcccttcttctcttagcgcaagcgtgggtgatcgggtcacaattacgtgccacgcgtctcaaaatatatacgtctggctgaactggtatcagcagaaaccaggaaaagcaccaaaactgcttatctataaagcaagcaatctccacactggggtacctagccgcttttccggcagcggctcaggcacagacttcaccttgactatttccagtctgcaacctgaggactttgctacatattattgtcaacagggtcaaacatacccctatactttcgggggggggactaaagtggaaattaagcgcaccgtaggctccacttcagggagcggcaaacctgggagcggcgaaggcagcactaagggacaagtccagcttgtgcaatctggagcagaggtcaagaaaccgggcgcctcagttaaggtctcctgtaaggctagcggatacacatttacttcctactatatacattgggtgcgacaagctccgggacaagggcttgaatggattggttgcatttaccctggaaatgtcaacaccaattacaacgaaaagtttaaggacagagccaccttgacggtggatacgtcaatatccactgcttacatggagctgagcagattgaggtccgatgatacggctgtttacttttgtactaggagtcattatgggcttgactggaactttgacgtatggggacagggtactaccgtcacagttagttcagaacagaaactgatcagtgaggaagatctgtttgtccctgtgtttcttcccgccaaacctactaccactcctgcacccaggccacccaccccagccccaacaattgcctctcaacctctctctctccgacctgaagcctgtaggccggctgctggcggagctgtacatacacgaggtctcgattttgcttgcgacatttatatttgggccccactcgccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 47 - DNA, 1431 bp, synthetic construct
  misc_feature 1..1431: note = CD28_CAR_11 encoding nucleotide sequence
  source 1..1431: mol_type = other DNA
atgttgctgttagttacatccttgctcctgtgcgaattgccacatccagctttccttctgatcccacaagtccagctggtgcaaagtggcgccgaggttaagaaacctggcgcatccgtgaaggtaagctgtaaggctagcggctacacctttacaagctactatattcattgggtgagacaagcaccaggacaaggactcgaatggataggctgcatttaccctggcaatgtaaacacgaattacaacgaaaagtttaaggaccgagccaccctgacagttgatactagcatcagtactgcgtacatggagcttagccgtcttaggagcgatgatactgcggtttacttttgtaccaggtcacattatgggcttgactggaactttgacgtctggggacagggaacaactgtcaccgtttcttccggttccacttcaggctccggtaaaccagggtctggcgaaggcagcaccaagggcgatatccaaatgacacaatctccctcatcattgagtgcaagtgtgggtgatcgcgtgactattacatgccacgcctctcaaaatatttacgtgtggctgaactggtatcagcagaaacccgggaaagcaccgaaactgctcatttataaagcatctaatttgcacaccggtgtaccctcaagatttagcggatcaggctcaggcacagacttcacactgacaatttcaagccttcaacccgaggactttgccacatattattgtcaacaggggcaaacatacccttatactttcggcggcgggacgaaagtggaaattaagcgcaccgttgaacagaaactgatctccgaggaagatctcggcggggggggatccggtggaggagggtccggaggaggtggttccatttatatttgggcaccactggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 48 - DNA, 1551 bp, synthetic construct
  misc_feature 1..1551: note = CD28_CAR_12 encoding nucleotide sequence
  source 1..1551: mol_type = other DNA
atgttgctgttagttacatccttgctcctgtgcgaactgccccatccagcgttcctgctgatcccccaagtccagctggtgcaaagtggcgccgaggtcaagaagccaggagccagcgttaaggtatcttgtaaggcatcaggctacacctttacatcttactatattcattgggtgagacaagctccgggacaagggttggaatggatagggtgcatataccctggtaatgtcaacactaattacaacgaaaagtttaaggaccgggccacactcactgtggatacgtcaatcagtacggcatacatggagctgagccggctccggagcgatgatacagcggtgtacttttgtacccgttcccattatgggctcgactggaactttgacgtatggggccaggggacaaccgttaccgtcagtagtggcagcacctcagggagcggtaaaccaggatctggcgaaggctccactaagggagatattcaaatgacgcaatcaccctcatcactgtctgccagtgtgggagatcgtgtcacgattacttgccacgcttctcaaaatatttacgtgtggttgaactggtatcagcaaaagcccgggaaagcaccaaaactcttgatctataaagcaagcaatctccacacaggggtcccgagccgattttctgggtccggcagtgggacagacttcacactgacgatttcatcactccaaccggaagactttgccacttattattgtcaacagggccaaacctacccctatactttcggcggcgggaccaaagtcgaaataaagcggaccgttgaacagaaattgatctccgaggaagatctgtttgtcccagtgtttcttcctgctaaaccaactactactcccgcccccagacccccgactcccgccccaactattgcttctcaacctttgagtctcagacccgaagcctgtaggccggccgccggtggggccgtccatactcgcggtttggattttgcatgcgacatttatatatgggcccctctggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 49 - DNA, 1545 bp, synthetic construct
  misc_feature 1..1545: note = CD28_CAR_14 encoding nucleotide sequence
  source 1..1545: mol_type = other DNA
atgttgctgttagttacatccttgctcctgtgcgaactgccccatcccgcgttccttctcatcccagtaaaacttcaacaatctggtgctgaattggtcaaaccaggggcttccgtacggctttcttgtaaagcttccggttatacttttacggaatatattatccattggataaaacttcgtagtggtcagggactcgagtggattggttggttttatcccggatccaatgatatacaatataatgcaaaatttaaagggaaagctactctcactgcagataagtcaagttccacagtctacatggaacttactggactcacatccgaagattctgctgtctacttttgtgcacggcgcgatgatttctctggatatgatgctcttccatattggggacaaggaacaatggtaacagtttcctctggtagcacttcaggatctggcaaaccgggttctggggaaggttccaccaagggcgatatacaaatgacacaaagtccagctagtctttccgtctccgtcggagaaacggtcacgattacgtgtcgaaccaatgaaaatatatattcaaatctcgcatggtatcaacaaaagcaaggaaaatctccacaacttctgatatatgcggcgactcatctcgttgaaggggtaccgtctcggttttcagggtccggatccggaacacaatatagtctcaaaataacaagtctccaatctgaggactttgggaattattattgtcaacatttctggggtactccatgtacatttggaggaggaactaaacttgaaataaaacgagaacagaaattgatatccgaggaagatcttttcgttcccgtgtttctccctgcaaaaccgacaactactcctgctccaaggccgcccactcccgcccctactatagcatcccaaccacttagtttgagacctgaagcctgtaggcccgccgcaggtggagctgtccatacacgcggcctcgattttgcttgcgacatttatatttgggcccctctggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 50 - RNA, 20 nt, synthetic construct
  misc_feature 1..20: note = spacer sequence of crRNA (targeting exon 2 of human CD28-gene)
  source 1..20: mol_type = other RNA
gcttgtagcgtacgacaatg

SEQ ID NO: 51 - RNA, 36 nt, synthetic construct
  misc_feature 1..36: note = crRNA (targeting exon 2 of human CD28-gene)
  source 1..36: mol_type = other RNA
gcttgtagcgtacgacaatggttttagagctatgct

SEQ ID NO: 52 - skipped

SEQ ID NO: 53 - protein, 14 aa, synthetic construct
  REGION 1..14: note = ITAM conserved motif
  VARIANT 2: note = any amino acid
  VARIANT 3: note = any amino acid
  VARIANT 4: note = Leu or Ile
  VARIANT 5: note = any amino acid
  VARIANT 6: note = any amino acid
  VARIANT 7: note = any amino acid
  VARIANT 8: note = any amino acid
  VARIANT 9: note = any amino acid
  VARIANT 10: note = Xaa or XaaXaa or XaaXaaXaa, wherein Xaa can be any amino acid
  VARIANT 12: note = any amino acid
  VARIANT 13: note = any amino acid
  VARIANT 14: note = Leu or Ile
YXXLXXXXXXYXXL

SEQ ID NO: 54 - DNA, 100 bp, synthetic construct
  misc_feature 1..100: note = 100 bp upstream of ATG start codon of pMP71
  source 1..100: mol_type = other DNA
tcgagtaatacgactcactatagggagacccaagctggctaggtaagcttgatcaacaagtttgtacaaaaaagcaggctccgcggccgcccccttcacc

SEQ ID NO: 55 - protein, 356 aa, synthetic construct
  REGION 1..356: note = truncated human epidermal growth factor receptor (EGFRt) epitope-tag (including signal peptide (amino acids 1-18))
LLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 56 - protein, 21 aa, synthetic construct
  REGION 1..21: note = T2A linker peptide
GSGEGRGSLLTCGDVEENPGP

SEQ ID NO: 57 - protein, 123 aa, synthetic construct
  REGION 1..123: note = VH of an anti-CD7 single-chain variable fragment (scFv)
QVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSS

SEQ ID NO: 58 - protein, 108 aa, synthetic construct
  REGION 1..108: note = VL of an anti-CD7 single-chain variable fragment (scFv)
DIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKR

SEQ ID NO: 59 - protein, 42 aa, synthetic construct
  source 1..42: note = human 4-1BB signaling domain
KRGRKKLLYIFKQPFMRPVQTTQEEDGCSCRFPEEEEGGCEL

SEQ ID NO: 60 - protein, 47 aa, synthetic construct
  source 1..47: note = murine 4-1BB signaling domain
VLKWIRKKFPHIFKQPFKKTTGAAQEEDACSCRCPQEEEGGGGGYEL

SEQ ID NO: 61 - protein, 68 aa, synthetic construct
  source 1..68: note = rat 4-1BB signaling domain
VLKNGTKEKDVVCGPPVVSLSPSTTPSAVTTPERESAFKKAVRTAQEEDACSCRFPEEEEGGGGSYEL

SEQ ID NO: 62 - protein, 182 aa, synthetic construct
  source 1..182: note = transmembrane and endodomain as comprised in construct CD28_CAR_21 and CD28_CAR_22
IYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 223 aa, synthetic construct
  source 1..223: note = transmembrane and endodomain as comprised in construct CD28_CAR_23 and CD28_CAR_24
IYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 517 aa, synthetic construct
  source 1..517: note = full-length amino acid sequence of construct CD28_CAR_21 including signal peptide
MLLLVTSLLLCELPHPAFLLIPDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVGSTSGSGKPGSGEGSTKGQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSEQKLISEEDLFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 515 aa, synthetic construct
  source 1..515: note = full-length amino acid sequence of construct CD28_CAR_22 including signal peptide
MLLLVTSLLLCELPHPAFLLIPVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSGSTSGSGKPGSGEGSTKGDIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKREQKLISEEDLFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 558 aa, synthetic construct
  source 1..558: note = full-length amino acid sequence of construct CD28_CAR_23 including signal peptide
MLLLVTSLLLCELPHPAFLLIPDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVGSTSGSGKPGSGEGSTKGQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSEQKLISEEDLFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 556 aa, synthetic construct
  source 1..556: note = full-length amino acid sequence of construct CD28_CAR_24 including signal peptide
MLLLVTSLLLCELPHPAFLLIPVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSSGSTSGSGKPGSGEGSTKGDIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKREQKLISEEDLFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 1554 bp, synthetic construct
  source 1..1554: mol_type = other DNA; note = nucleotide sequence encoding construct CD28_CAR_21 including signal peptide
atgttgctgttagttacatccttgctcctgtgcgaactcccacatcccgcttttctcctgatcccagatattcaaatgacccaaagcccttcttctcttagcgcaagcgtgggtgatcgggtcacaattacgtgccacgcgtctcaaaatatatacgtctggctgaactggtatcagcagaaaccaggaaaagcaccaaaactgcttatctataaagcaagcaatctccacactggggtacctagccgcttttccggcagcggctcaggcacagacttcaccttgactatttccagtctgcaacctgaggactttgctacatattattgtcaacagggtcaaacatacccctatactttcgggggggggactaaagtggaaattaagcgcaccgtaggctccacttcagggagcggcaaacctgggagcggcgaaggcagcactaagggacaagtccagcttgtgcaatctggagcagaggtcaagaaaccgggcgcctcagttaaggtctcctgtaaggctagcggatacacatttacttcctactatatacattgggtgcgacaagctccgggacaagggcttgaatggattggttgcatttaccctggaaatgtcaacaccaattacaacgaaaagtttaaggacagagccaccttgacggtggatacgtcaatatccactgcttacatggagctgagcagattgaggtccgatgatacggctgtttacttttgtactaggagtcattatgggcttgactggaactttgacgtatggggacagggtactaccgtcacagttagttcagaacagaaactgatcagtgaggaagatctgtttgtccctgtgtttcttcccgccaaacctactaccactcctgcacccaggccacccaccccagccccaacaattgcctctcaacctctctctctccgacctgaagcctgtaggccggctgctggcggagctgtacatacacgaggtctcgattttgcttgcgacatttatatttgggccccactcgccgggacttgtggggtccttctcctgtcactggttatcaccctttactgcaaccacaggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 69 - DNA, 1548 bp, synthetic construct
  source 1..1548: mol_type = other DNA; note = nucleotide sequence encoding construct CD28_CAR_22 including signal peptide
atgttgctgttagttacatccttgctcctgtgcgaactgccccatcccgcgttccttctcatcccagtaaaacttcaacaatctggtgctgaattggtcaaaccaggggcttccgtacggctttcttgtaaagcttccggttatacttttacggaatatattatccattggataaaacttcgtagtggtcagggactcgagtggattggttggttttatcccggatccaatgatatacaatataatgcaaaatttaaagggaaagctactctcactgcagataagtcaagttccacagtctacatggaacttactggactcacatccgaagattctgctgtctacttttgtgcacggcgcgatgatttctctggatatgatgctcttccatattggggacaaggaacaatggtaacagtttcctctggtagcacttcaggatctggcaaaccgggttctggggaaggttccaccaagggcgatatacaaatgacacaaagtccagctagtctttccgtctccgtcggagaaacggtcacgattacgtgtcgaaccaatgaaaatatatattcaaatctcgcatggtatcaacaaaagcaaggaaaatctccacaacttctgatatatgcggcgactcatctcgttgaaggggtaccgtctcggttttcagggtccggatccggaacacaatatagtctcaaaataacaagtctccaatctgaggactttgggaattattattgtcaacatttctggggtactccatgtacatttggaggaggaactaaacttgaaataaaacgagaacagaaattgatatccgaggaagatcttttcgttcccgtgtttctccctgcaaaaccgacaactactcctgctccaaggccgcccactcccgcccctactatagcatcccaaccacttagtttgagacctgaagcctgtaggcccgccgcaggtggagctgtccatacacgcggcctcgattttgcttgcgacatttatatttgggcccctctggccgggacttgtggggtccttctcctgtcactggttatcaccctttactgcaaccacaggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 70 - DNA, 1677 bp, synthetic construct
  source 1..1677: mol_type = other DNA; note = nucleotide sequence encoding construct CD28_CAR_23 including signal peptide
atgttgctgttagttacatccttgctcctgtgcgaactcccacatcccgcttttctcctgatcccagatattcaaatgacccaaagcccttcttctcttagcgcaagcgtgggtgatcgggtcacaattacgtgccacgcgtctcaaaatatatacgtctggctgaactggtatcagcagaaaccaggaaaagcaccaaaactgcttatctataaagcaagcaatctccacactggggtacctagccgcttttccggcagcggctcaggcacagacttcaccttgactatttccagtctgcaacctgaggactttgctacatattattgtcaacagggtcaaacatacccctatactttcgggggggggactaaagtggaaattaagcgcaccgtaggctccacttcagggagcggcaaacctgggagcggcgaaggcagcactaagggacaagtccagcttgtgcaatctggagcagaggtcaagaaaccgggcgcctcagttaaggtctcctgtaaggctagcggatacacatttacttcctactatatacattgggtgcgacaagctccgggacaagggcttgaatggattggttgcatttaccctggaaatgtcaacaccaattacaacgaaaagtttaaggacagagccaccttgacggtggatacgtcaatatccactgcttacatggagctgagcagattgaggtccgatgatacggctgtttacttttgtactaggagtcattatgggcttgactggaactttgacgtatggggacagggtactaccgtcacagttagttcagaacagaaactgatcagtgaggaagatctgtttgtccctgtgtttcttcccgccaaacctactaccactcctgcacccaggccacccaccccagccccaacaattgcctctcaacctctctctctccgacctgaagcctgtaggccggctgctggcggagctgtacatacacgaggtctcgattttgcttgcgacatttatatttgggccccactcgccgggacttgtggggtccttctcctgtcactggttatcaccctttactgcaaccacaggaacaggtccaaaagaagtcgactgctccatagcgattatatgaatatgaccccgagacggcctggaccaactcgtaaacactatcaaccttacgcaccaccccgggattttgctgcatacaggagtaagagaggacggaagaagctgctctacatctttaagcagcccttcatgcggcccgtgcagacgacccaggaagaggacggatgcagttgtaggttcccggaagaggaagagggtggctgcgagctccgagttaaattttcacggtcagccgatgcacctgcttatcaacaaggacaaaatcaactgtacaatgaactgaacctgggccgccgggaagaatatgacgtcctcgataaaaggcgaggaagagatccagaaatgggtgggaaaccacgacggaagaatccgcaagaggggctctataacgagttgcaaaaggacaaaatggccgaagcatatagcgaaatcggtatgaagggtgaacggcgtcgcgggaaaggccatgacggactgtatcaaggcttgtctactgctacaaaagatacgtatgatgctctgcatatgcaagccctgccccctcgctaa

SEQ ID NO: 71 - DNA, 1671 bp, synthetic construct
  source 1..1671: mol_type = other DNA; note = nucleotide sequence encoding construct CD28_CAR_24 including signal peptide
atgttgctgttagttacatccttgctcctgtgcgaactgccccatcccgcgttccttctcatcccagtaaaacttcaacaatctggtgctgaattggtcaaaccaggggcttccgtacggctttcttgtaaagcttccggttatacttttacggaatatattatccattggataaaacttcgtagtggtcagggactcgagtggattggttggttttatcccggatccaatgatatacaatataatgcaaaatttaaagggaaagctactctcactgcagataagtcaagttccacagtctacatggaacttactggactcacatccgaagattctgctgtctacttttgtgcacggcgcgatgatttctctggatatgatgctcttccatattggggacaaggaacaatggtaacagtttcctctggtagcacttcaggatctggcaaaccgggttctggggaaggttccaccaagggcgatatacaaatgacacaaagtccagctagtctttccgtctccgtcggagaaacggtcacgattacgtgtcgaaccaatgaaaatatatattcaaatctcgcatggtatcaacaaaagcaaggaaaatctccacaacttctgatatatgcggcgactcatctcgttgaaggggtaccgtctcggttttcagggtccggatccggaacacaatatagtctcaaaataacaagtctccaatctgaggactttgggaattattattgtcaacatttctggggtactccatgtacatttggaggaggaactaaacttgaaataaaacgagaacagaaattgatatccgaggaagatcttttcgttcccgtgtttctccctgcaaaaccgacaactactcctgctccaaggccgcccactcccgcccctactatagcatcccaaccacttagtttgagacctgaagcctgtaggcccgccgcaggtggagctgtccatacacgcggcctcgattttgcttgcgacatttatatttgggcccctctggccgggacttgtggggtccttctcctgtcactggttatcaccctttactgcaaccacaggaacaggtccaaaagaagtcgactgctccatagcgattatatgaatatgaccccgagacggcctggaccaactcgtaaacactatcaaccttacgcaccaccccgggattttgctgcatacaggagtaagagaggacggaagaagctgctctacatctttaagcagcccttcatgcggcccgtgcagacgacccaggaagaggacggatgcagttgtaggttcccggaagaggaagagggtggctgcgagctccgagttaaattttcacggtcagccgatgcacctgcttatcaacaaggacaaaatcaactgtacaatgaactgaacctgggccgccgggaagaatatgacgtcctcgataaaaggcgaggaagagatccagaaatgggtgggaaaccacgacggaagaatccgcaagaggggctctataacgagttgcaaaaggacaaaatggccgaagcatatagcgaaatcggtatgaagggtgaacggcgtcgcgggaaaggccatgacggactgtatcaaggcttgtctactgctacaaaagatacgtatgatgctctgcatatgcaagccctgccccctcgctaa